(19) United States
(12) Patent Application Publication
(10) Pub. No.: US 2016/0176914 A1
(43) Pub. Date: Jun. 23, 2016

(54) GLYCOSYLATED CARDIOTONIC STEROIDS

(71) Applicants: NORTHEASTERN UNIVERSITY, Boston, MA (US); JOHNS HOPKINS TECHNOLOGY VENTURES, Baltimore, MD (US)

(72) Inventors: George A. O'Doherty, Boston, MA (US); Hongyan Li, Boston, MA (US); Sumit O. Bajaj, Boston, MA (US); Hua-Yu Leo Wang, Dallas, TX (US); Michael F. Cuccarese, Cambridge, MA (US); Ravit Boger, Baltimore, MD (US)

(21) Appl. No.: 14/787,383

(22) PCT Filed: May 29, 2014
(86) PCT No.: PCT/US2014/039989

§ 371 Date: Oct. 27, 2015

Related U.S. Application Data
(60) Provisional application No. 61/828,231, filed on May 29, 2013.
(60) Provisional application No. 61/828,277, filed on May 29, 2013.

(51) Int. Cl. C07J 19/00

(52) U.S. Cl. CPC C07J 19/005

(57) ABSTRACT

Compounds which are glycosylates of an A-ring of a cardiotonic steroid, wherein the steroid is attached to the anomeric position of (a) a monosaccharide comprising a C-4 amino group, or (b) an oligosaccharide are provided.

### CROSS-REFERENCE TO RELATED APPLICATIONS

This PCT application claims the benefit of priority to U.S. Provisional Patent Applications 61/828,231, filed May 29, 2013 and 61/828,277, filed May 29, 2013, the entire disclosures of which are hereby incorporated by reference in their entirety for any and all purposes.

### FIELD

The present technology generally relates to biologically active novel cardiotonic steroid compounds. In particular, the present technology is related to novel glycosylated cardiotonic steroids and methods for preparing and using them.

### BACKGROUND

Cardiotonic steroids (CSs) have been widely used for the treatment of heart failure and as chemotherapeutic agents in oncology. Cardiotonic steroids, are known to bind with high specificity and affinity to the Na⁺/K⁺-transporting ATPase, which is used by the cardiotonic steroids as a signal transducer to activate tissue proliferation, heart contractility, arterial hypertension and natriuresis, via various intra-cellular signalling pathways. Plants containing cardiotonic steroids have long been known for their medicinal use. Digitoxin, is a clinically approved glycosylated cardiotonic steroid for heart failure has shown anti-cancer effect in several types of cancer. Chemically, glycosylated CSs are compounds having a steroid nucleus with a lactone moiety at position 17 and a sugar moiety at position 3 as shown below:

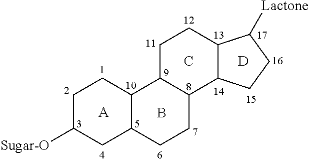

A few methods are known for preparation of cardiac glycoside analogs by glycosylation of aglycon compounds such as e.g., Digitoxigenin. However, most methods fail to place the functionalized sugar with the desired streospecificity and often result in products which do not exhibit the desired level of activity. There is, therefore, a need for glycosylated cardiotonic steroids having improved pharmacological activity, cytotoxicity and pharmacokinetic properties. The present invention aims to provide novel glycosylated cardiotonic steroids having these desired properties and methods for preparing these glycosylated compounds.

### SUMMARY

In one aspect, novel glycosylates of cardiotonic steroids are provided. In one aspect, provided is a compound which is a glycosylate of an A-ring of a cardiotonic steroid, wherein the steroid is attached to the anomeric position of (a) a monosaccharide comprising a C-4 amino group, or (b) an oligosaccharide.

In another aspect, provided are novel glycosylates of cardiotonic steroids represented by formula I:

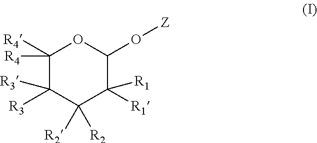

wherein:
each of R₁, R₁′, R₂, and R₂′ independently, is H, OH, alkyl, alkenyl, alkynyl, aryl, alkoxyl, cycloalkyl, heterocycloalkyl, heteroaryl, O-aryl, O-monosaccharide, O-oligosaccharide;
each of R₃ and R₃′, independently, is H, OH, alkyl, alkoxyl, cycloalkyl, alkenyl, alkynyl, aryl, cycloalkyl, heterocycloalkyl, heteroaryl, O-aryl, O-monosaccharide, O-oligosaccharide, and NR₂₀R₂₁, R₂₀ and R₂₁ being H, alkyl, alkenyl, alkynyl, aryl, cycloalkyl, heterocycloalkyl, heteroaryl;
each of R₄ and R₄′, independently, is H, alkyl, alkenyl, alkynyl, aryl, alkoxyl, cycloalkyl, heterocycloalkyl, and heteroaryl, or R₄ and R₄′ together with the carbon atom they attach to form a cycloalkyl or heterocycloalkyl ring; and
Z is a cardiotonic steroid aglycon.

In some embodiments, R₃ is H or alkyl and R₃′ is NR₂₀R₂₁. In some embodiments, R₃′ is NH₂. In some embodiments, R₁ is H or OH. In some embodiments, R₂ is H, OH, or O-monosaccharide. In some embodiments, R₂ or R₂′ is O-monosaccharide, 0-disaccharide, or O-trisaccharide. In some embodiments, R₂ or R₂′ is O-monosaccharide. In some embodiments, R₁ or R₁′ is O-monosaccharide, 0-disaccharide, or O-trisaccharide. In some embodiments, R₁ or R₁′ is O-monosaccharide.

In some embodiments, Z is represented by formula II:

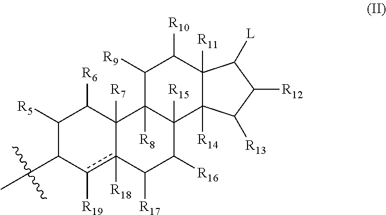

wherein:
[symbol] indicates a single or a double bond;
each of R₅, R₆, R₇, R₈, R₁₁, R₁₃, R₁₄, R₁₅, R₁₆, R₁₇, R₁₈, and R₁₉, independently, is H, OH, carbonyl, alkyl, alkoxyl, acyloxy, carboxy, alkylcarboxy, hydroxyalkyl, —C(O)R₂₂, or two adjacent groups together with the bond that the two groups attach to form an epoxide ring;
R₂₂ is H or alkyl;
each of R₉, R₁₀, and R₁₂, independently, is H, OH, carbonyl, or a cleavable prodrug group; and
L is a heterocyclic ring.

In some embodiments, R₇ is H, —OH, CH₃, CH₂OH, C═O, C(O)H, —OC(O)H, or —OC(O)alkyl. In some embodiments, R₁₀ is H, —OH, CH₃, or CH₂OH. In some embodiments, R_{H} is H, —OH, CH₃, or CH₂OH. In some embodiments, R₁₄ is H, OH, or together with R₁₃ forms a three membered epoxy ring.

In some embodiments, each of R₉, R₁₀, and R₁₂, independently, is a cleavable prodrug group. In some embodiments, each of R₁₀ is a cleavable prodrug group. In some embodiments, the cleavable prodrug group comprises ethers, esters, carbonates, carbamates, sugars, sulfates and phosphates. In some embodiments, the cleavable prodrug group is a pivaloyl, trialkylsilane, acetyl, or chloroacetyl group.

In some embodiments, L is a lactone. In some embodiments, L is represented by formula III:

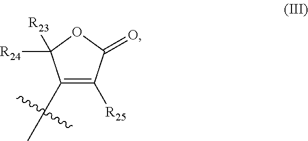

wherein:

each of R₂₃, R₂₄, and R₂₅, independently, is H, halo, alkyl, alkenyl, alkynyl, alkoxyl, cycloalkyl, aryl, carboxy, alkylcarboxy, amino, alkylamino, or dialkylamino.

For compounds of formula III, in some embodiments, each of R₂₃, R₂₄, and R₂₅ is H. In some embodiments, Z is digitoxigenin, digoxigenin, lanatoside B aglycon, gitoformate aglycon, oleandrigenin, k-strophanthidin, cannogenin, bipindogenin, g-strophanthidin (ouabagenin), periplogenin, or uzarigenin aglycon.

In some embodiments, L is represented by formula IV:

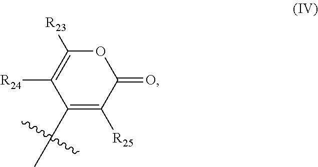

wherein:
each of R₂₃, R₂₄, and R₂₅, independently, is H, halo, alkyl, alkenyl, alkynyl, alkoxyl, cycloalkyl, aryl, carboxy, alkylcarboxy, amino, alkylamino, or dialkylamino.

For compounds of formula IV, in some embodiments, each of R₂₃, R₂₄, and R₂₅ is H. In some embodiments, Z is arenobufagin, bufalin, bufatonin, telocinobufagin, cinobufagin, marinobufgin, proscillaridin aglycon, or scilliroside agylcon.

In some embodiments, Z is digitoxigenin. In other embodiments, Z is digoxigenin. In some embodiments, Z is other than digitoxin, oleandrin, digitoxin-β-D-digitoxose, or digitoxin-mono-α-L-rhamnoside.

In yet another aspect, a pharmaceutical composition is provided, which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the composition, the compound is represented by formula I described herein.

In one aspect, a method of treating congestive heart failure in a subject is provided, the method including administering to the subject a pharmaceutical composition which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the method, the compound is represented by formula I described herein.

In another aspect, a method of treating cancer in a subject is provided, the method including administering to the subject a pharmaceutical composition which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the method, the compound is represented by formula I described herein.

In yet another aspect, a method of treating a viral infection in a subject comprising administering to the subject a pharmaceutical composition which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the method, the compound is represented by formula I described herein.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIG. 1 illustrates the basic structural motifs of the novel glycosylated cardiotonic steroid compounds of the present technology.

FIG. 2 illustrates exemplary cardiac glycoside aglycons of the novel glycosylated cardiotonic steroid compounds of the present technology.

FIG. 3 illustrates exemplary bicyclic cardiac glycoside aglycons of the novel glycosylated cardiotonic steroid compounds of the present technology.

FIG. 4 shows the dose response curve for H460 cells for exemplary glycosylated cardiotonic steroid compounds of the present technology.

FIG. 5 shows the dose response curve for H460 cells for a exemplary glycosylated cardiotonic steroid prodrug compounds of the present technology.

### DETAILED DESCRIPTION

### Definitions

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of (+) or (−) 20 percent, 10 percent, 5 percent or 1 percent.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The term sugar residue as used herein may refer to any residue derived from a sugar. The term “sugar” used herein may be interpreted to mean that it includes sugars, carbohydrates, saccharides, complex sugars, sugar conjugates and other sugar-related compounds. As used herein, the term “saccharide” refers to a single sugar moiety or monosaccharide unit as well as combinations of two or more single sugar moieties or monosaccharide units covalently linked to form disaccharides, oligosaccharides, and polysaccharides. The term “saccharide” may be used interchangeably with the terms “carbohydrate.” The saccharide may be linear or branched.

A “monosaccharide” as used herein refers to a single sugar residue in an oligosaccharide. The term “disaccharide” as used herein refers to a saccharide composed of two monosaccharide units or moieties linked together by a glycosidic bond. In one embodiment, the saccharide is an oligosaccharide. An “oligosaccharide” as used herein refers to a compound containing two or more monosaccharide units or moieties. Within the context of an oligosaccharide, an individual monomer unit or moiety is a monosaccharide, which is, or can be, bound through a hydroxyl group to another monosaccharide unit or moiety.

In general, “substituted” refers to an alkyl, alkenyl, alkynyl, aryl, or ether group, as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

As used herein, Cₘ-Cₙ, such as C₁-C₁₂, C₁-C₈, or C₁-C₆ when used before a group refers to that group containing m to n carbon atoms.

As used herein, “alkyl” groups include straight chain and branched alkyl groups having from 1 to about 20 carbon atoms (i.e., C₁-C₂₀ alkyl), and typically from 1 to 12 carbons (i.e., C₁-C₁₂ alkyl) or, in some embodiments, from 1 to 8 carbon atoms (i.e., C₁-C₈ alkyl). As employed herein, “alkyl groups” include cycloalkyl groups as defined below. Alkyl groups may be substituted or unsubstituted. Examples of straight chain alkyl groups include methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups may be substituted one or more times with, for example, amino, thio, hydroxy, cyano, alkoxy, and/or halo groups such as F, Cl, Br, and I groups. As used herein the term haloalkyl is an alkyl group having one or more halo groups. In some embodiments, haloalkyl refers to a per-haloalkyl group. Alkyl groups defined herein also include haloalkyl, polyhaloalkyl, perfluoroalkyl, hydroxyalkyl, polyhydroxyalkyl and haloalkyl.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups may be substituted or unsubstituted. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to: 2,2-; 2,3-; 2,4-; 2,5-; or 2,6-disubstituted cyclohexyl groups or mono-, di-, or tri-substituted norbornyl or cycloheptyl groups, which may be substituted with, for example, alkyl, alkoxy, amino, thio, hydroxy, cyano, and/or halo groups. Cycloalkyl groups defined herein also include polyhalocycloalkyl, perfluorocycloalkyl, hydroxycycloalkyl and polyhydroxycycloalkyl.

Alkenyl groups are straight chain, branched or cyclic alkyl groups having 2 to about 20 carbon atoms, and further including at least one double bond. In some embodiments alkenyl groups have from 1 to 12 carbons, or, typically, from 1 to 8 carbon atoms. Alkenyl groups may be substituted or unsubstituted. Alkenyl groups include, for instance, vinyl, propenyl, 2-butenyl, 3-butenyl, isobutenyl, cyclohexenyl, cyclopentenyl, cyclohexadienyl, butadienyl, pentadienyl, and hexadienyl groups among others. Alkenyl groups may be substituted similarly to alkyl groups. Divalent alkenyl groups, i.e., alkenyl groups with two points of attachment, include, but are not limited to, CH—CH═CH₂, C═CH₂, or C═CHCH₃.

The term “alkynyl” as used herein, means a straight or branched chain hydrocarbon group containing from 2 to 6 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, “aryl”, or “aromatic,” groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. The phrase “aryl groups” includes groups containing fused rings, such as fused aromatic-aliphatic ring systems (e.g., indanyl, tetrahydronaphthyl, and the like). Aryl groups may be substituted or unsubstituted.

The term “halo” refers to F, Cl, Br, and/or I.

The term “heteroaryl” refers to a monovalent, aromatic mono-, bi-, or tricyclic ring having 2-16 ring carbon atoms and 1-8 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

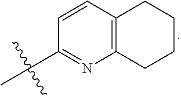 [SMILES: *c1ccc2c(n1)CCCC2]

The term “heterocycloalkyl” or “heterocyclyl” or heterocycle refers to a non-aromatic, mono-, bi-, or tricyclic ring containing 2-12 ring carbon atoms and 1-8 ring heteroatoms selected preferably from N, O, S, and P and oxidized forms of N, S, and P, provided that the ring contains at least 3 ring atoms. While heterocycloalkyl preferably refers to saturated ring systems, it also includes ring systems containing 1-3 double bonds, provided that the ring is non-aromatic. Nonlimiting examples of heterocycloalkyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocycloalkyl group. For example, and without limitation, the following is a heterocycloalkyl group:

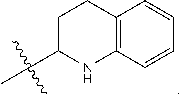 [SMILES: *C1CCc2ccccc2N1]

Unless otherwise specifically defined, a lactone is a cyclic ester having 4 to 8 ring members. The lactone can be unsubstituted, singly substituted or, if possible, multiply substituted, with substituent groups in any possible position.

An “effective amount”, “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration of an affliction, or one or more symptoms thereof, prevent the advancement of conditions related to an affliction, prevent the recurrence, development, or onset of one or more symptoms associated with an affliction, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

The term “in need thereof” refers to the need for symptomatic or asymptomatic relief from a condition.

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in “Remington's Pharmaceutical Sciences” by E. W. Martin (herein incorporated by reference in its entirety).

The term “composition(s)” or “composition(s) of the technology” as used herein means compositions comprising any of compounds described herein, or salts, tautomeric forms, hydrates, and solvates thereof.

The term “method(s)” or “method(s) of the technology” as used herein means methods comprising treatment with the compounds and/or compositions of the technology.

The term “solvate” as used herein means a compound, or a pharmaceutically acceptable salt thereof, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

As used herein, a “prodrug” is a compound that, after administration, is metabolized or otherwise converted to an active or more active form with respect to at least one property. To produce a prodrug, a pharmaceutically active compound can be modified chemically to render it less active or inactive, but the chemical modification is such that an active form of the compound is regenerated by biological or chemical processes. A prodrug may have, relative to the drug, altered metabolic stability or transport characteristics, improved pharmacokinetic or pharmacological properties, fewer side effects or lower toxicity. A prodrug is an active drug chemically transformed into a per se inactive derivative which by virtue of chemical or bilological action is converted to the parent drug within the body of the subject before or after reaching the site of action.

A “pharmaceutical composition” refers to a mixture of one or more of the compounds described herein, or pharmaceutically acceptable salts, tautomers, solvates, or hydrates thereof, with other chemical components, such as physiologically acceptable carriers and excipients. The purpose of a pharmaceutical composition is to facilitate administration of a compound to an organism.

The term “pharmaceutically acceptable” refers to safe and non-toxic for in vivo, preferably, human administration.

The term “pharmaceutically acceptable salt” is intended to include salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluroacetic, trichloroacetic, naphthalene-2 sulfonic, oxalic, propionic, and other acids. Salts may also exist as solvates or hydrates. Other exemplary pharmaceutically acceptable salts are described herein.

As used herein, the terms “animal,” “subject” and “patient” as used herein include all members of the animal kingdom including, but not limited to, mammals, animals (e.g., cats, dogs, horses, swine, etc.) and humans. In some embodiments, an “individual” refers to a human. In some embodiments, an “animal” refers to, for example, nonhuman-primates such as monkeys and baboons; veterinary animals, such as rodents, dogs, cats, horses and the like; and farm animals, such as cows, pigs and the like. In some embodiments, the subject or patient is a human.

In various embodiments, the compounds disclosed herein may suitably include isomers, pharmaceutically acceptable salts, solvates, hydrates, amides, esters, ethers, chemically protected forms, tautomers, polymorphs, and prodrugs thereof. In various embodiments, the glycosylated cardiotonic steroids described herein encompass all isomers including enantiomers, diastereomers, geometric isomers, racemates, tautomers, rotamers, and atropisomers, N-oxides, salts, solvates, and/or hydrates, metabolites, and pharmaceutically acceptable salts. In general, the compositions of the technology may be alternately formulated to comprise, consist of, or consist essentially of, any appropriate components herein disclosed. The compositions of the technology may additionally, or alternatively, be formulated so as to be devoid, or substantially free, of any components, materials, ingredients, adjuvants or species or that are otherwise not necessary to the achievement of the function and/or objectives of the present technology.

### Compounds

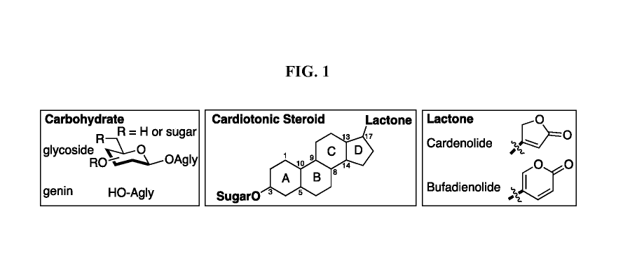

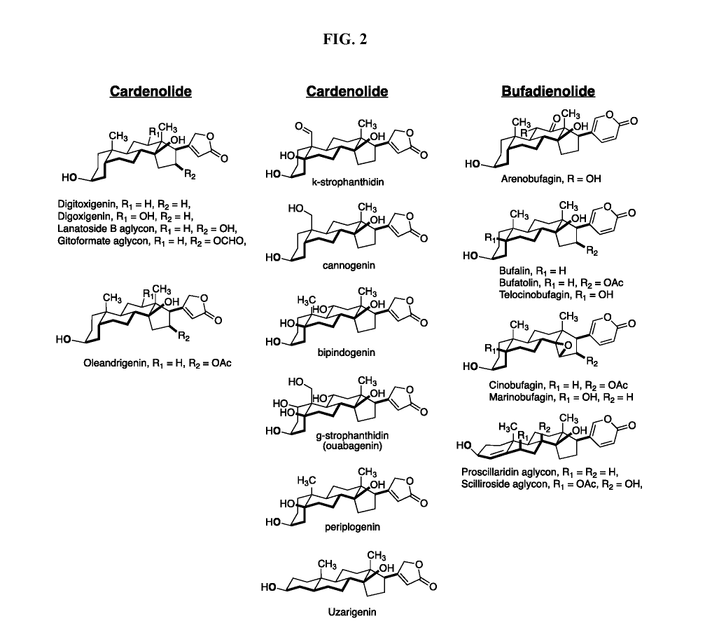

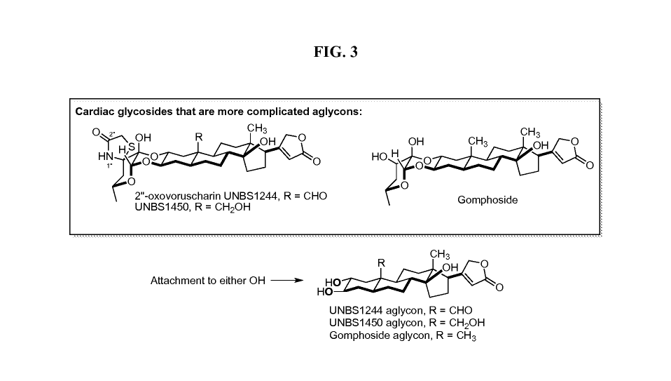

The present technology is generally directed to glycosylated steroid derivatives. In one aspect, the invention is directed to the synthesis and identification of novel glycosylated cardiac glycosides. The basic structural motifs of the novel glycosylated cardiotonic steroids of the invention are depicted in FIG. 1. The cardiotonic glycoside aglycons suitable for compounds of the present invention include Cardenolides and Bufadienolides. Suitable glycoside compounds include those having one or more than one attachment points on the aglycon for carbohydrate substitution. The cardiac glycoside aglycons of the present compounds with desirable sites for carbohydrate substitution are depicted in FIG. 2. The cardiac glycosides with two attachment points to the aglycon are depicted in FIG. 3. Suitable sugar molecules attached to the steroid are known in the art and include, but are not limited to rhamnose, amicetose, mycinose, vallarose, fucose, quinovose, anarose, oliose, digitose, boivinose, oleandrose, rhodinose, ascarylose and the like. The sugar molecule may be of D- or L-configuration and may include mono-, mono-, di-, tri-, or oligosaccharides.

In one embodiment, the novel compounds of the invention are glycosylated at the A-ring of the cardiotonic steroidal framework and selectively improve its Na⁺/K⁺-ATPase inhibitory activity. The substitution on the sugar molecule is configured so that it can be used to selectively inhibit the various α-isoforms of the Na⁺/K⁺-ATPase pump, which in turn can lead to the selective targeting of the specific tissues and disease sites (e.g., cancer tumors, heart). Additionally, the novel glycosylated compounds may also exhibit improved pharmacokinetic properties (e.g., improved metabolic stability, solubility and membrane permeability), which in turn renders the compounds better drugs for the treatment of various diseases, e.g., via the inhibition of the Na⁺/K⁺-ATPase pump (e.g., congestive heart failure, cancer, viral infections etc.). In one embodiment, the sugar molecule attached to the A-ring of the cardiotonic steroid can be of any stereochemical configuration and can have various substituents.

In another embodiment, the novel compounds of the invention are substituted at various positions on the C and D-ring of the cardiotonic steroidal framework, e.g., with prodrug moieities such as ethers, esters, carbonates, carbamates, sugars, sulfates and phosphates, and the like. The substitution on the C and D-ring can be configured to control the Na⁺/K⁺-ATPase pump inhibitory activity in a tunable fashion. This technology is used to design prodrug molecules for the selective treatment of diseases that are susceptible to inhibitors of the Na⁺/K⁺-ATPase pump, such as heart disease (e.g., congestive heart failure), cancer and viral infections (e.g., infection with the cytomegalovirus (CMV)). In addition to controlling inhibitory activity, the substitution can also be used to selectively target specific tissues and disease sites (e.g., photodynamic therapy, hypoxia activated prodrugs). In one embodiment, the positions of the C and D-ring of the cardiotonic steroid can be of either alpha or beta configuration. The mechanism of cleavage of the various prodrug moietites or cleavable prodrug groups can include any known method, e.g., via biological, chemical, biochemical, photochemical, or change in pH induced transformation.

In one aspect, novel glycosylates of cardiotonic steroids are provided. In one aspect, provided is a compound which is a glycosylate of an A-ring of a cardiotonic steroid, wherein the steroid is attached to the anomeric position of (a) a monosaccharide comprising a C-4 amino group, or (b) an oligosaccharide.

In another aspect, provided are novel glycosylates of cardiotonic steroids represented by formula I:

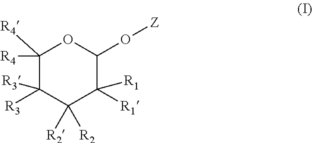

wherein:
each of R₁, R₁′, R₂, and R₂′ independently, is H, OH, alkyl, alkenyl, alkynyl, aryl, alkoxyl, cycloalkyl, heterocycloalkyl, heteroaryl, O-aryl, O-monosaccharide, O-oligosaccharide;
each of R₃ and R₃′, independently, is H, OH, alkyl, alkoxyl, cycloalkyl, alkenyl, alkynyl, aryl, cycloalkyl, heterocycloalkyl, heteroaryl, O-aryl, O-monosaccharide, O-oligosaccharide, and NR₂₀R₂₁, R₂₀ and R₂₁ being H, alkyl, alkenyl, alkynyl, aryl, cycloalkyl, heterocycloalkyl, heteroaryl;
each of R₄ and R₄′, independently, is H, alkyl, alkenyl, alkynyl, aryl, alkoxyl, cycloalkyl, heterocycloalkyl, and heteroaryl, or R₄ and R₄′ together with the carbon atom they attach to form a cycloalkyl or heterocycloalkyl ring; and
Z is a cardiotonic steroid aglycon.

In some embodiments, R₁ is H or OH. In some embodiments, R₁ or R₁′ is O-monosaccharide, 0-disaccharide, or O-trisaccharide. In some embodiments, R₁ or R₁′ is O-monosaccharide.

In some embodiments, R₂ is H, OH, or O-monosaccharide. In some embodiments, R₂ or R₂′ is O-monosaccharide, 0-disaccharide, or O-trisaccharide. In some embodiments, R₂ or R₂′ is O-monosaccharide. In some embodiments, R₂ is H. In other embodiments, R₂ is OH. In some embodiments, R₂′ is O-monosaccharide. In some embodiments, R₂ or R₂′ is selected from the group consisting of D-rhamnose, L-amicetose, D-amicetose, (1,3)-L,D-Dirhamnose, and (1,3)-L,L-Dirhamnose.

In some embodiments, R₃ is H or alkyl and R₃′ is NR₂₀R₂₁. In some embodiments, R₃ is H. In some embodiments, R₃ is CH₃. In some embodiments, R₃′ is NH₂. In some embodiments, R₃′ is NH—CH₃. In some embodiments, R₃′ is N(CH₃)₂.

For compounds of formula (I), Z can be any suitable aglycon (i.e. cardiotonic steroid nucleus together with a lactone) known in the art. Exemplary aglycons are also depicted in FIG. 2 and FIG. 3.

In some embodiments, the cardiotonic steroid aglycon Z is represented by formula II:

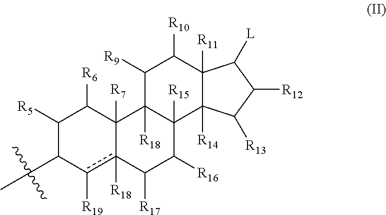

wherein:
[symbol] indicates a single or a double bond;
each of R₅, R₆, R₇, R₈, R₁₁, R₁₃, R₁₄, R₁₅, R₁₆, R₁₇, R₁₈, and R₁₉, independently, is H, OH, carbonyl, alkyl, alkoxyl, acyloxy, carboxy, alkylcarboxy, hydroxyalkyl, —C(O) R₂₂, or two adjacent groups together with the bond that the two groups attach to form an epoxide ring;
R₂₂ is H or alkyl;
each of R₉, R₁₀, and R₁₂, independently, is H, OH, carbonyl, or a cleavable prodrug group; and
L is a heterocyclic ring.

In some embodiments, [symbol] indicates a single bond, In other embodiments, [symbol] indicates a double bond. In some embodiments, when [symbol] indicates a double bond, R₁₈ and R₁₉ are H. In some embodiments, R₁₈ is H and R₁₉ are alkyl.

In some embodiments, R⁷ is H, —OH, CH₃, CH₂OH, C═O, C(O)H, —OC(O)H, or —OC(O)alkyl. In some embodiments, R¹⁰ is H, —OH, CH₃, or CH₂OH. In some embodiments, R¹¹ is H, —OH, CH₃, or CH₂OH. In some embodiments, R¹⁴ is H, OH, or together with R¹³ forms a three membered epoxy ring.

In some embodiments, Z is selected from the group consisting of digitoxigenin, digoxigenin, lanatoside B aglycon, gitoformate aglycon, oleandrigenin, k-strophanthidin, cannogenin, bipindogenin, g-strophanthidin (ouabagenin), periplogenin, and uzarigenin aglycon. In other embodiments, Z is selected from the group consisting of arenobufagin, bufalin, bufatonin, telocinobufagin, cinobufagin, marinobufgin, proscillaridin aglycon, and scilliroside agylcon.

In some embodiments, Z is digitoxigenin. In other embodiments, Z is digoxigenin. In some embodiments, Z is other than digitoxin, oleandrin, digitoxin-β-D-digitoxose, or digitoxin-mono-α-L-rhamnoside.

In some embodiments, the cardiotonic steroid aglycon Z is represented by formula IIA:

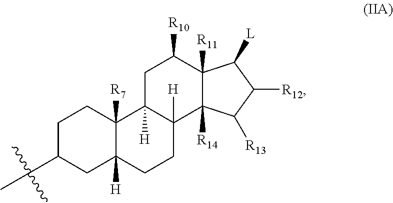

wherein the substituents are as defined herein.

In some embodiments, the hydroxyl groups on the steroid molecule are substituted with a cleavable molecule. This includes substitution on all four rings. In some embodiments, the hydroxyl groups on ring C and D of the steroid moiety are substituted with one or more cleavable prodrug groups. With these substituents the glycosylated cardiotonic steroids can function as a prodrug with the cleavable prodrug groups serving as a linker.

In some embodiments, each of R₉, R₁₀, and R₁₂, independently, is a cleavable prodrug group. Suitable cleavable prodrug groups are known in the art. These include groups which can be chemically attached to the OH groups on the steroid moiety to form a stable compound, and which can be cleaved by any known method, e.g., via biological, chemical, biochemical, photochemical or change in pH induced transformation. In some embodiments, R₁₀ is a cleavable prodrug group. In some embodiments, the cleavable prodrug group comprises ethers, esters, carbonates, carbamates, sugars, sulfates and phosphates. In some embodiments, the cleavable prodrug group is a pivaloyl, trialkylsilane, acetyl, or chloroacetyl group. In some embodiments, the cleavable prodrug group is tert-butyl-dimethylsilyl ether (TBSO). In some embodiments, the cleavable prodrug group is a nitobenzyl carbonate group. In some embodiments, the cleavable prodrug group is 4-nitobenzyl carbonate.

For compounds of formula II, L is a heterocyclic ring. In some embodiments, L represents a lactone, for example, a five-membered lactone ring (e.g., an unsaturated butyrolactone ring) or a six-membered lactone ring (e.g., an α-pyrone ring).

In some embodiments, L is represented by formula III:

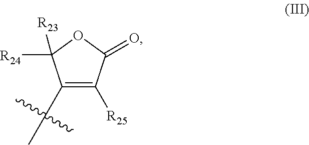

wherein:
each of R₂₃, R₂₄, and R₂₅, independently, is H, halo, alkyl, alkenyl, alkynyl, alkoxyl, cycloalkyl, aryl, carboxy, alkylcarboxy, amino, alkylamino, or dialkylamino.

In some embodiments, L is represented by formula IV:

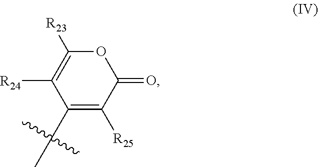

wherein:
each of R₂₃, R₂₄, and R₂₅, independently, is H, halo, alkyl, alkenyl, alkynyl, alkoxyl, cycloalkyl, aryl, carboxy, alkylcarboxy, amino, alkylamino, or dialkylamino.

For compounds of formula III and IV, in some embodiments, R₂₃ is H. In some embodiments, R₂₃ is CH₃. In some embodiments, R₂₃ is OCH₃. In some embodiments, R₂₄ is H. In some embodiments, R₂₄ is CH₃. In some embodiments, R₂₄ is OCH₃. In some embodiments, R₂₅ is H. In some embodiments, R₂₅ is CH₃. In some embodiments, R₂₅ is OCH₃. In some embodiments, each of R₂₃, R₂₄, and R₂₅ is H.

In some embodiments, a compound is provided; wherein the compound is selected from the group consisting of digitoxigenin α-L-rhamnopyranoside; digitoxigenin α-L-amecitopyranoside; digoxigenin α-L-rhamnopyranoside; digoxigenin α-L-amecitopyranoside; digitoxigenin α-L-rhamnopyranosyl-(1→3)-α-L-rhamnopyranoside; digitoxigenin α-L-rhamnopyranosyl-(1→3)-α-L-rhamnopyranosyl-(1→3)-α-L-rhamnopyranoside; digitoxigenin α-L-4-amino-rhamnopyranoside; digitoxigenin α-L-4-amino-amecitopyranoside; digitoxigenin α-L-rhamnopyranosyl-(1→3)-α-L-rhamnopyranosyl-(1→3)-α-L-rhamnopyranosyl-(1→3)-α-L-rhamnopyranoside; digitoxigenin α-L-rhamnopyranosyl-(1→3)-α-L-4-amino-rhamnopyranoside; digitoxigenin α-L-4-amino-rhamnopyranosyl-(1→3)-α-L-rhamnopyranoside; digitoxigenin α-L-4-amino-rhamnopyranosyl-(1→3)-α-L-4-amino-rhamnopyranoside; digitoxigenin α-L-4-azido-rhamnopyranoside; digitoxigenin α-L-4-azido-amecitopyranoside; digitoxigenin α-L-rhamnopyranosyl-(1→3)-α-L-4-azido-rhamnopyranoside; digitoxigenin α-L-4-azido-rhamnopyranosyl-(1→3)-α-L-rhamnopyranoside; digitoxigenin α-L-4-azido-rhamnopyranosyl-(1→3)-α-L-4-azido-rhamnopyranoside; digitoxigenin α-D-rhamnopyranosyl-(1→3)-α-L-rhamnopyranoside; digitoxigenin α-D-rhamnopyranosyl-(1→3)-α-L-rhamnopyranosyl-(1→3)-α-L-rhamnopyranoside; digitoxigenin α-D-rhamnopyranosyl-(1→3)-α-L-rhamnopyranosyl-(1→3)-α-L-rhamnopyranosyl-(1→3)-α-L-rhamnopyranoside; digitoxigenin α-D-rhamnopyranosyl-(1→4)-α-L-rhamnopyranoside; digitoxigenin α-D-amecitopyranosyl-(1→4)-α-L-rhamnopyranoside; digitoxigenin α-D-rhamnopyranosyl-(1→4)-α-L-amecitopyranoside; digitoxigenin α-L-4-amino-rhamnopyranosyl-(1→4)-α-D-digitoxopyranosyl-(1→4)-α-D-digitoxopyranosyl-(1→4)-α-D-digitoxopyranopyranoside; digitoxigenin α-L-4-amino-amecitopyranosyl-(1→4)-α-D-digitoxopyranosyl-(1→4)-α-D-digitoxopyranosyl-(1→4)-α-D-digitoxopyranopyranoside; digitoxigenin α-D-4-amino-rhamnopyranosyl-(1→4)-α-D-digitoxopyranosyl-(1→4)-α-D-digitoxopyranosyl-(1→4)-α-D-digitoxopyranopyranoside; digitoxigenin α-D-4-amino-amecitopyranosyl-(1→4)-α-D-digitoxopyranosyl-(1→4)-α-D-digitoxopyranosyl-(1→4)-α-D-digitoxopyranoside; digitoxigenin α-L-4-azido-rhamnopyranosyl-(1→4)-α-D-digitoxopyranosyl-(1→4)-α-D-digitoxopyranosyl-(1→4)-α-D-digitoxopyranoside; digitoxigenin α-L-4-azido-amecitopyranosyl-(1→4)-α-D-digitoxopyranosyl-(1→4)-α-D-digitoxopyranosyl-(1→4)-α-D-digitoxopyranoside; digitoxigenin α-D-4-azido-rhamnopyranosyl-(1→4)-α-D-digitoxopyranosyl-(1→4)-α-D-digitoxopyranosyl-(1→4)-α-D-digitoxopyranoside; or digitoxigenin α-D-4-azido-amecitopyranosyl-(1→4)-α-D-digitoxopyranosyl-(1→4)-α-D-digitoxopyranosyl-(1→4)-α-D-digitoxopyranoside.

Exemplary compounds of the technology and their structures are provided in the table below.

| Compd. | | |
|---|---|---|
| No. | Compound Name | Structure |
| 1 | digitoxigenin α-L- rhamnopyranoside | 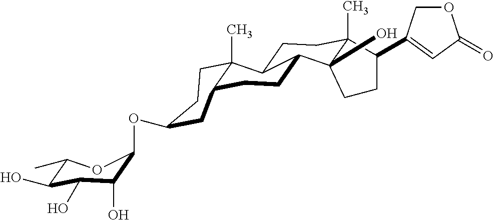 |
| 2 | digitoxigenin α-L- amecitopyranoside | 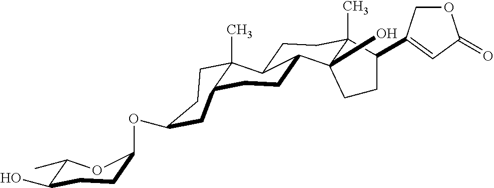 |
| 3 | digoxigenin α-L- rhamnopyranoside | 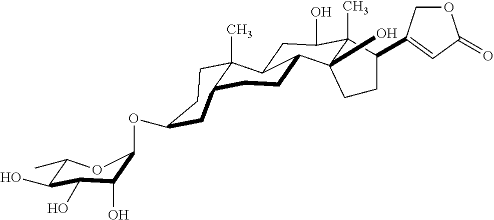 |
| 4 | digoxigenin α-L- amecitopyranoside | 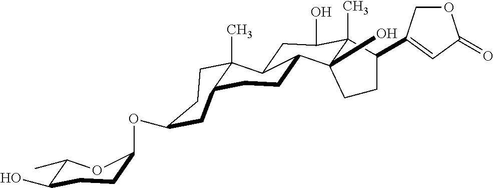 |
| 5 | digitoxigenin α-L- rhamnopyranosyl- (1 → 3)-α-L- rhamnopyranoside | 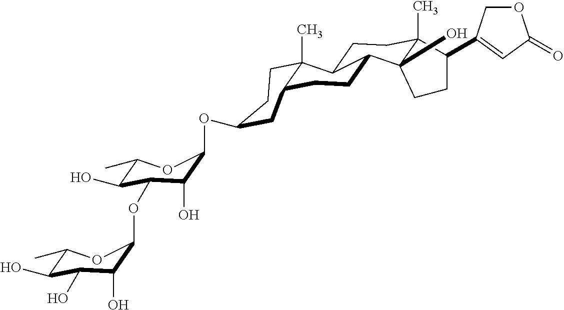 |
| 6 | digitoxigenin α-L- rhamnopyranosyl- (1 → 3)-α-L- rhamnopyranosyl- (1 → 3)-α-L- rhamnopyranoside | 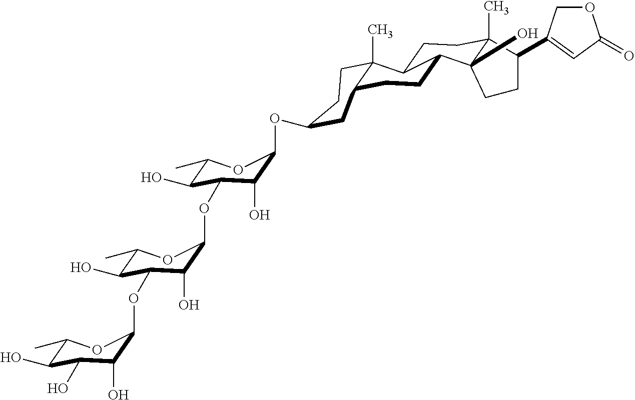 |
| 7 | digitoxigenin α-L-4- amino- rhamnopyranoside | 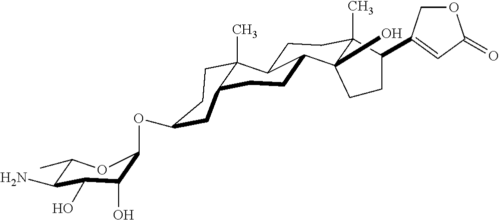 |
| 8 | digitoxigenin α-L-4- amino- amecitopyranoside | 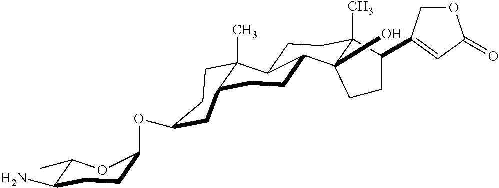 |
| 9 | digitoxigenin α-L- rhamnopyranosyl- (1 → 3)-α-L- rhamnopyranosyl- (1 → 3)-α-L- rhamnopyranosyl- (1 → 3)-α-L- rhamnopyranoside | 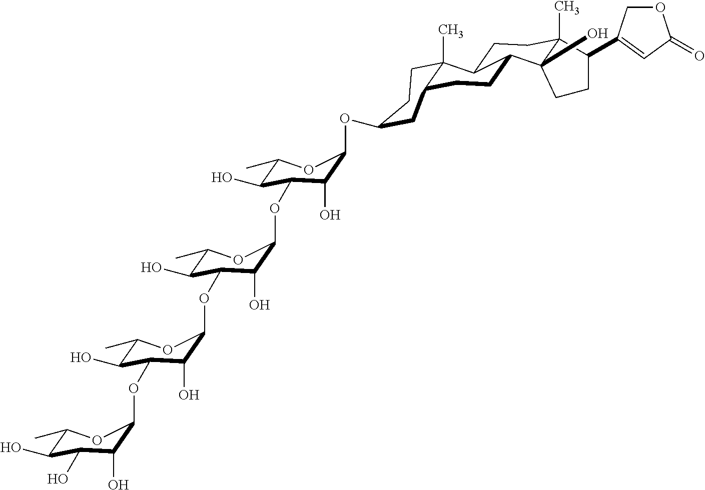 |
| 10 | digitoxigenin α-L- rhamnopyranosyl- (1 → 3)-α-L-4-amino- rhamnopyranoside | 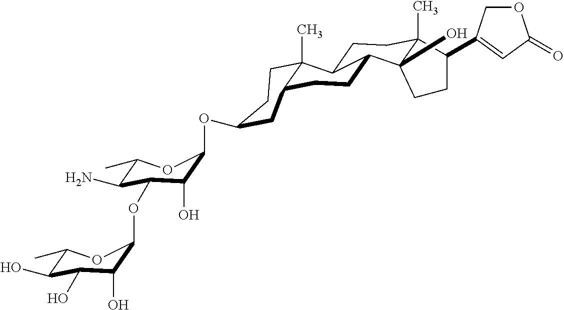 |
| 11 | digitoxigenin α-L-4- amino- rhamnopyranosyl- (1 → 3)-α-L- rhamnopyranoside | 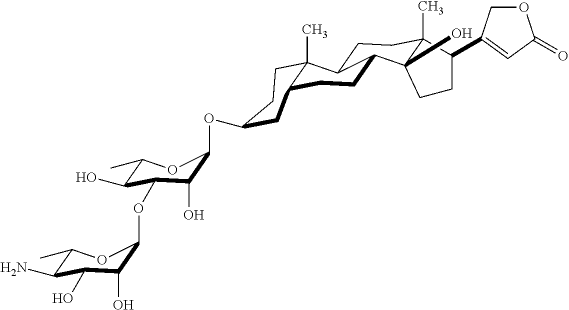 |
| 12 | digitoxigenin α-L-4- amino- rhamnopyranosyl- (1 → 3)-α-L-4-amino- rhamnopyranoside | 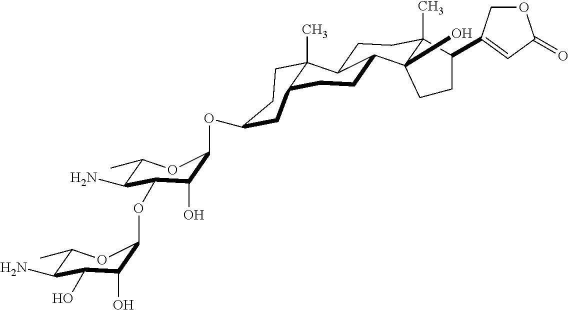 |
| 13 | digitoxigenin α-L-4- azido- rhamnopyranoside | 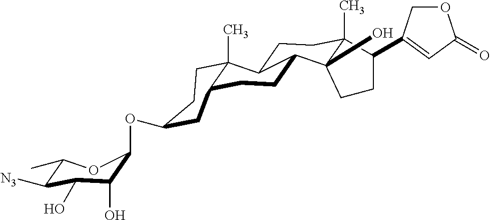 |
| 14 | digitoxigenin α-L-4- azido- amecitopyranoside | 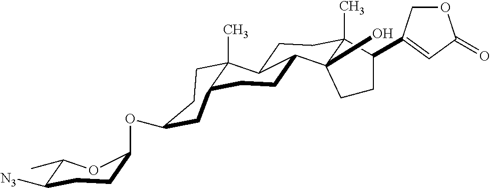 |
| 15 | digitoxigenin α-L- rhamnopyranosyl- (1 → 3)-α-L-4-azido- rhamnopyranoside | 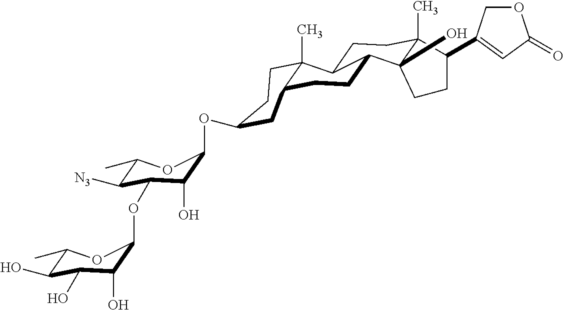 |
| 16 | digitoxigenin α-L-4- azido- rhamnopyranosyl- (1 → 3)-α-L- rhamnopyranoside | 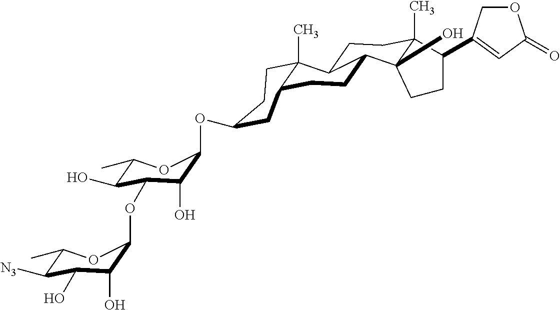 |
| 17 | digitoxigenin α-L-4- azido- rhamnopyranosyl- (1 → 3)-α-L-4-azido- rhamnopyranoside | 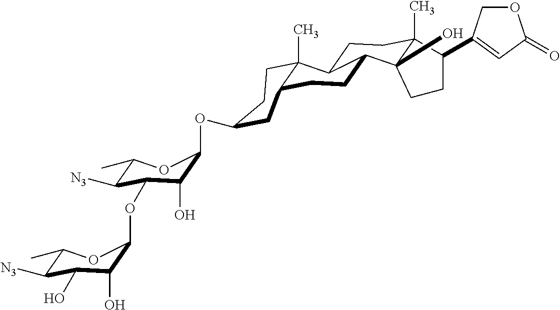 |
| 18 | digitoxigenin α-D- rhamnopyranosyl- (1 → 3)-α-L- rhamnopyranoside | 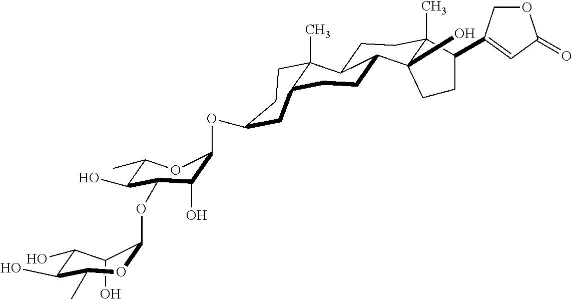 |
| 19 | digitoxigenin α-D- rhamnopyranosyl- (1 → 3)-α-L- rhamnopyranosyl- (1 → 3)-α-L- rhamnopyranoside | 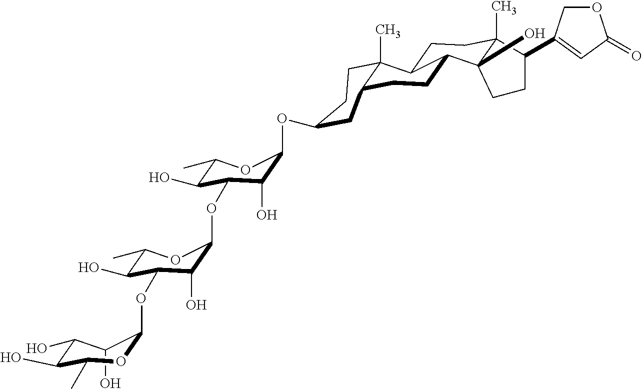 |
| 20 | digitoxigenin α-D- rhamnopyranosyl- (1 → 3)-α-L- rhamnopyranosyl- (1 → 3)-α-L- rhamnopyranosyl- (1 → 3)-α-L- rhamnopyranoside | 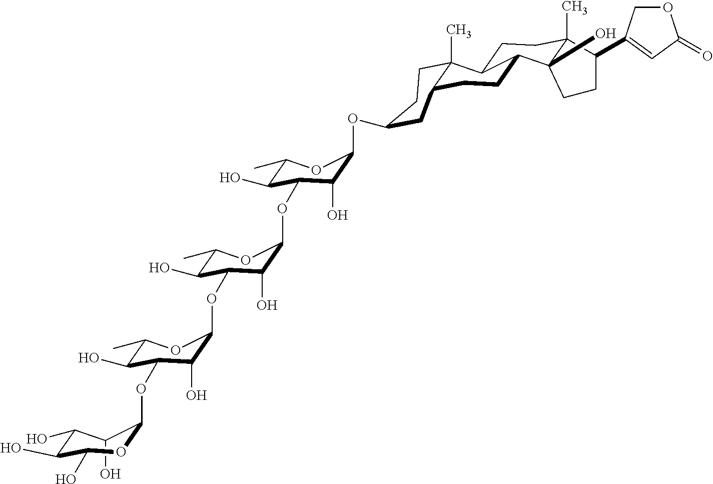 |
| 21 | digitoxigenin α-D- rhamnopyranosyl- (1 → 4)-α-L- rhamnopyranoside | 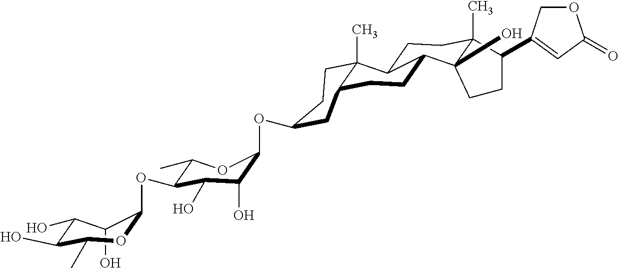 |
| 22 | digitoxigenin α-D- amecitopyranosyl- (1 → 4)-α-L- rhamnopyranoside | 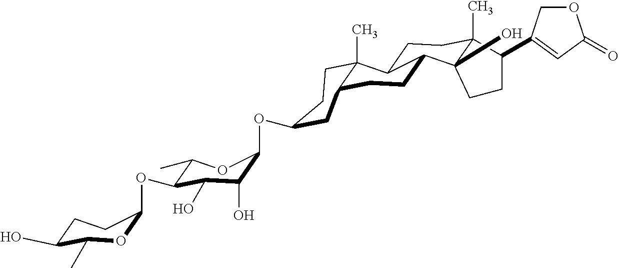 |
| 23 | digitoxigenin α-L- rhamnopyranosyl- (1 → 4)-α-L- amecitopyranoside | 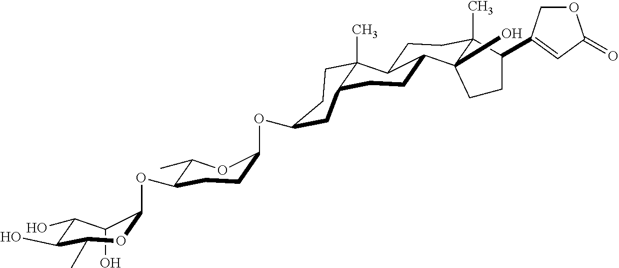 |
| 24 | digitoxigenin α-L-4- amino- rhamnopyranosyl- (1 → 4)-α-D- rhamnopyranosyl- (1 → 4)-α-D- digitoxopyranosyl- (1 → 4)-α-D- digitoxopyranoside | 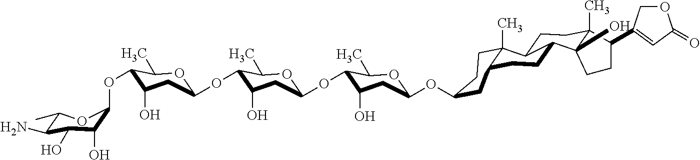 |
| 25 | digitoxigenin α-L-4- amino- amecitopyranosyl- (1 → 4)-α-D- digitoxopyranosyl- (1 → 4)-α-D- digitoxopyranosyl- (1 → 4)-α-D- digitoxopyranoside | 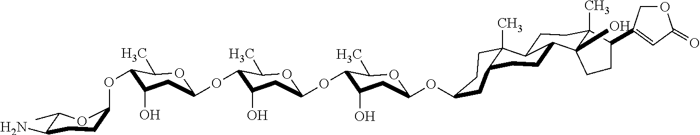 |
| 26 | digitoxigenin α-D-4- amino- rhamnopyranosyl- (1 → 4)-α-D- digitoxopyranosyl- (1 → 4)-α-D- digitoxopyranosyl- (1 → 4)-α-D- digitoxopyranoside | 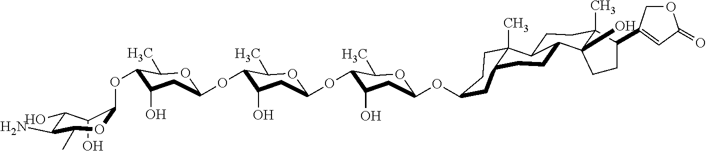 |
| 27 | digitoxigenin α-D-4- amino- amecitopyranosyl- (1 → 4)-α-D- digitoxopyranosyl- (1 → 4)-α-D- digitoxopyranosyl- (1 → 4)-α-D- digitoxopyranoside | 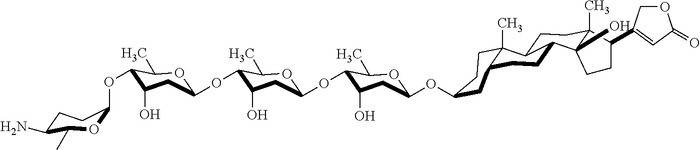 |
| 28 | digitoxigenin α-L-4- azido- rhamnopyranosyl- (1 → 4)-α-D- digitoxopyranosyl- (1 → 4)-α-D- digitoxopyranosyl- (1 → 4)-α-D- digitoxopyranoside | 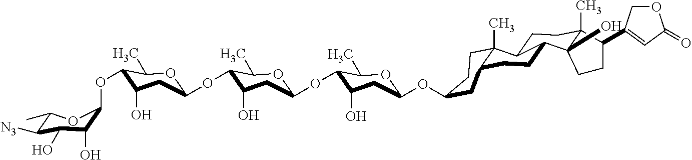 |
| 29 | digitoxigenin α-L-4- azido- amecitopyranosyl- (1 → 4)-α-D- digitoxopyranosyl- (1 → 4)-α-D- digitoxopyranosyl- (1 → 4)-α-D- digitoxopyranoside | 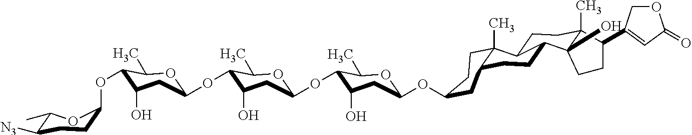 |
| 30 | digitoxigenin α-D-4- azido- rhamnopyranosyl- (1 → 4)-α-D- digitoxopyranosyl- (1 → 4)-α-D- digitoxopyranosyl- (1 → 4)-α-D- digitoxopyranoside | 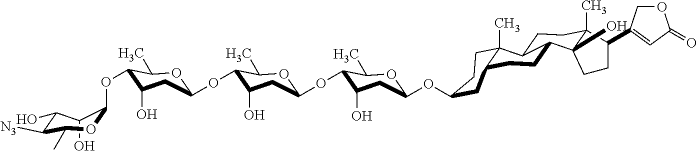 |
| 31 | digitoxigenin α-D-4- azido- amecitopyranosyl- (1 → 4)-α-D- digitoxopyranosyl- (1 → 4)-α-D- digitoxopyranosyl- (1 → 4)-α-D- digitoxopyranoside | 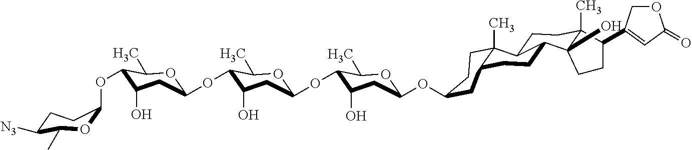 |

### Pharmaceutical Compositions

In further aspects of the technology, a composition is provided comprising any of the compounds described herein, and at least a pharmaceutically acceptable excipient. In another aspect, this invention provides a composition comprising any of the compounds described herein, and a pharmaceutically acceptable excipient

In one aspect, a pharmaceutical composition is provided, which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the composition, the compound is represented as formula I described herein.

Pharmaceutically acceptable excipients are non-toxic, aid administration, and do not adversely affect the therapeutic benefit of the compound. Such excipients may be any solid, liquid, semi-solid or, in the case of an aerosol composition, gaseous excipient that is generally available to one of skill in the art. Pharmaceutical compositions in accordance with the technology are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin, and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. In certain embodiments, the compositions provided herein comprises one or more of α-tocopherol, gum arabic, and/or hydroxypropyl cellulose.

In some embodiments, the glycosylated CS compounds are formulated into pharmaceutical compositions for administration to subjects in a biologically compatible form suitable for administration in vivo. According to another aspect, the present technology provides a pharmaceutical composition comprising a glycosylated CS compound described herein in admixture with a pharmaceutically acceptable diluent and/or carrier. The pharmaceutically-acceptable carrier is “acceptable” in the sense of being compatible with the other ingredients of the composition and not deleterious to the recipient thereof. The pharmaceutically-acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans. When administered to a subject, the compound and pharmaceutically acceptable carrier can be sterile. Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain minor amounts of wetting or emulsifying agents, or pH buffering agents.

Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N+R′R″R′″R″″Y−, in which the R radicals are identical or different optionally hydroxylated hydrocarbon radicals and Y- is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine and the like.

The pharmaceutical formulations of the present technology are prepared by methods well-known in the pharmaceutical arts. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

A pharmaceutical composition of the technology is formulated to be compatible with its intended route of administration. Exemplary routes include, but are not limited to oral, transdermal, intravenous, intraarterial, pulmonary, rectal, nasal, vaginal, lingual, intramuscular, intraperitoneal, intracutaneous, intracranial, and subcutaneous routes. Suitable dosage forms for administering any of the compounds described herein include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16th ed., A. Oslo editor, Easton Pa. 1980).

The compounds and/or compositions of the present technology are administered to a human or animal subject by known procedures including oral administration, sublingual or buccal administration. In some embodiments, the compound or composition is administered orally. In some embodiments, the composition may be administered via direct injection.

For oral administration, a formulation of the compounds of the technology may be presented in dosage forms such as capsules, tablets, powders, granules, or as a suspension or solution. Capsule formulations may be gelatin, soft-gel or solid. Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants.

Compounds and pharmaceutical compositions described herein may be used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the glycosylate CS compound described herein and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present technology.

### Methods

### Therapeutic Methods

The pharmacological actions of glycosylated cardiotonic compounds of the present technology are mediated through interaction with the sodium pump, Na⁺/K⁺-ATPase (NKA). It is understood that the as specific inhibitors of membrane-bound Na⁺/K⁺-ATPase, the glycosylated CSs enhance cardiac contractility through increasing myocardial cell calcium in response to the resulting increase in intracellular Na. The correlation between Na⁺/K⁺-ATPase modulators and anticancer effects has also been investigated, for example, by Wang, H.-Y. L.; and O'Doherty, G. A. in Expert Opin. Therapeutic Patents, 2012, 22, 587-605.

Thus in one aspect, a method of inhibiting a Na⁺1 K⁺-ATPase pump is provided, the method including contacting the Na⁺/K⁺-ATPase pump with a glycosylate of an A-ring of a cardiotonic steroid, wherein the steroid is attached to the anomeric position of (a) a monosaccharide comprising a C-4 amino group, or (b) an oligosaccharide. In some embodiments of the method, the compound is represented as formula I described herein.

In light of the Na⁺/K⁺-ATPase pump inhibiting activity, the compounds of the present technology can be used in treating, preventing or controlling the indications or diseases associated with such an activity. In some embodiments, the glycosylated CS compounds of the present technology can be used in the treatment of congestive heart failure as positive inotropic agents, as anti-cancer agents, as anti-hypertensive agents, and as antiviral agents (e.g., anti-cytomegalovirus activity).

In one aspect, a method of treating congestive heart failure in a subject is provided, the method including administering to the subject a pharmaceutical composition which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the method, the compound is represented as formula I described herein.

In another aspect, a method of treating cancer in a subject is provided, the method including administering to the subject a pharmaceutical composition which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the method, the compound is represented as formula I described herein.

In yet another aspect, provided is a method of treating a viral infection in a subject comprising administering to the subject a pharmaceutical composition which includes a pharmaceutically acceptable excipient and a compound which is a glycosylate of an A-ring of a cardiotonic steroid, wherein the steroid is attached to the anomeric position of (a) a monosaccharide comprising a C-4 amino group, or (b) an oligosaccharide. In some embodiments of the method, the compound is represented as formula I described herein.

In some embodiments, the novel compounds described herein have improved anti-human Cytomegalovirus (HCMV) activities. In some embodiments the compounds described herein inhibit HCMV replication at very low concentrations. Accordingly, in one embodiment, a method of inhibiting CMV replication in a subject in need of such treatment is provided, which includes administering to the patient an amount of the compound of formula I as described herein.

The compounds described herein by virtue of the cleavable prodrug groups attached to the the steroid component are tunable, such that their activity can be turned on or turned off selectively as desired. In some embodiments, the novel compounds described herein, especially the prodrug compounds, can be used in tumor-selective or cancer-selective treatment wherein the inactive prodrug can be administered to the subject, which can be cleaved and rendered active selectively in the tumor environment only. For example, the novel prodrug compounds of the present technology having ester groups as the cleavable prodrug group can be used for targeted tissue treatment, wherein the ester is hydrolysed, and selectively activated by an enzyme which is coupled to an antibody which is specific for said target tissue. In some embodiments, the antibody can be specific for cancer cells. In some embodiments, the prodrug ester compound can be hydrolyzed and activated endogeneous or exogeneous enzyme.

In another aspect, provided is a method of treating a hypoxic condition in a subject which includes administering to the patient an amount of the compound of formula I as described herein. Suitable compounds of formula I would include those having a cleavable prodrug group on the steroid moiety. Preferably, the hypoxic condition is cancer or cardiac disease. The prodrug compounds can administered in an inactive form but which become activated in a hypoxic environment.

### Synthetic Methods

Certain methods for making the compounds described herein are also provided. The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

The compounds of the present technology can be synthesized using various methods known in the art and the procedures depicted in Schemes 1-6 and discussed in the Examples.

The present technology, thus generally described, will be understood more readily by reference to the following examples, which are provided by way of illustration and are not intended to be limiting of the present invention.

### EXAMPLES

### Example 1

### Synthesis of Digoxin Monosaccharides

The digoxin monosaccharide compounds were synthesized using the procedure depicted in Scheme 1:

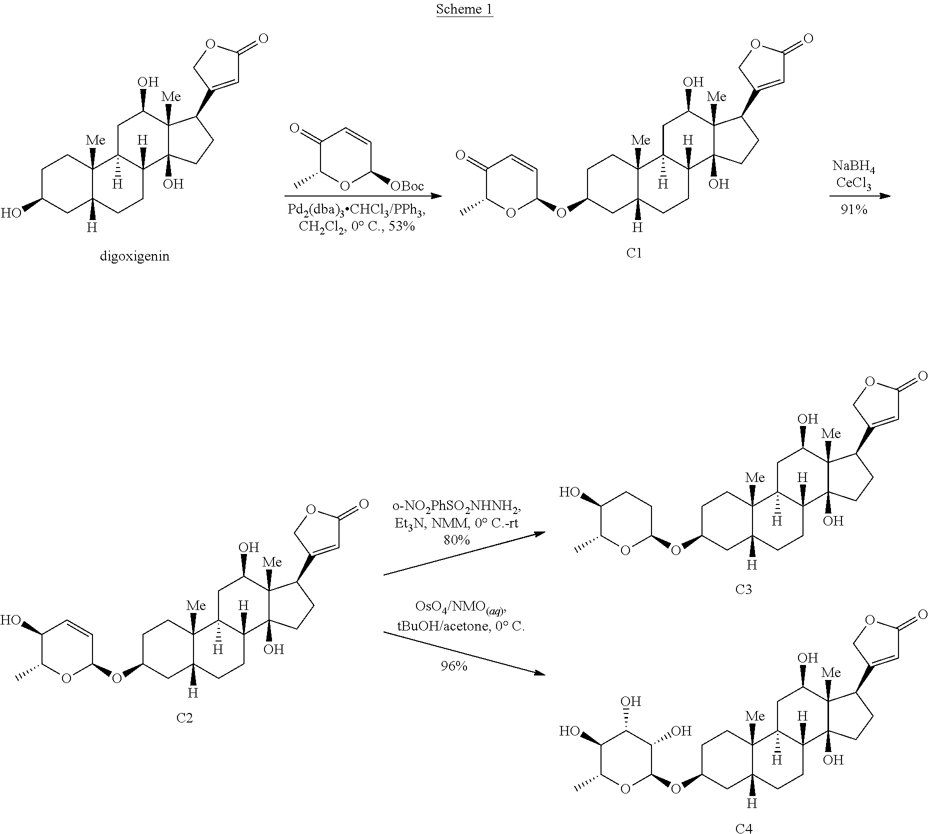

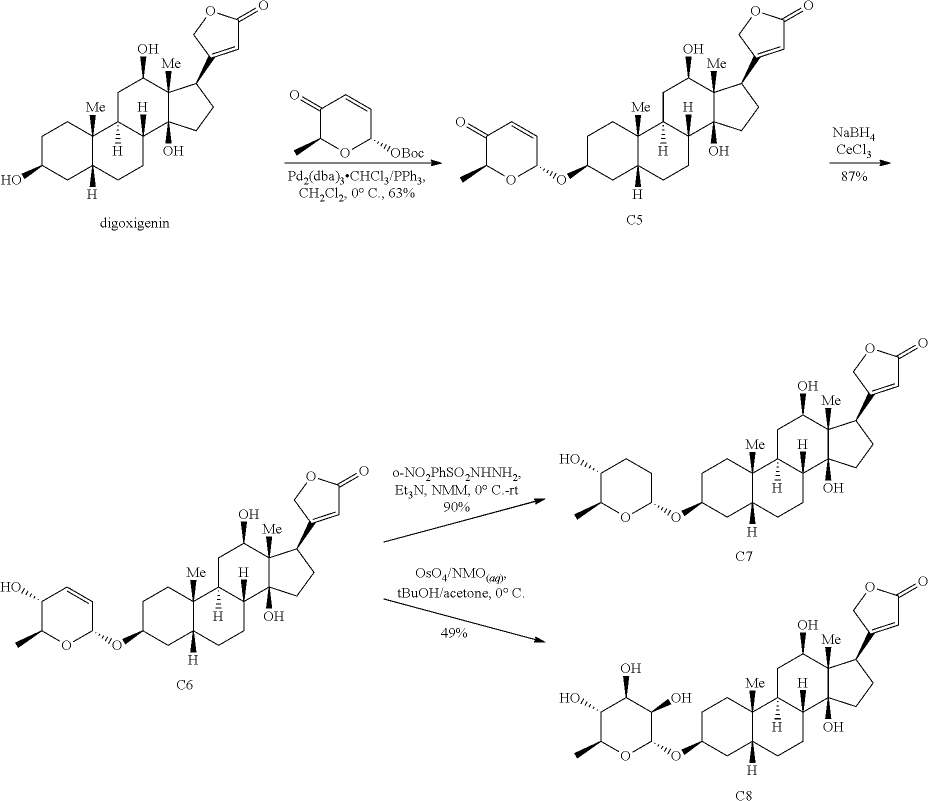

Synthesis of C1:

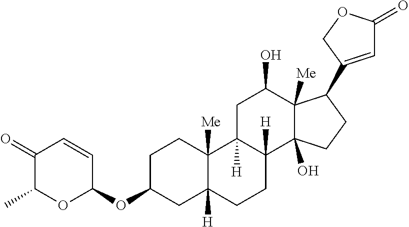

781 mg digoxigenin and 1.52 g tert-butyl ((2R,6R)-6-methyl-5-oxo-5,6-dihydro-2H-pyran-2-yl) carbonate was dissolved in THF/CH₂Cl₂ (1:1, 20 ml/20 ml), and cooled to 0° C. 83 mg Pd₂(DBA)₃.CHCl₃ and 84 mg Ph₃P was dissolved in 8 ml CH₂Cl₂ and added to the above mixture at 0° C. The reaction mixture was stirred at 0° C. for 18 hours, and then was quenched by saturated NaHCO₃ solution and extracted by EtOAc, dried over Na₂SO₄, and concentrated. The crude product was purified by flash chromatography with 40% EtOAc in hexanes to afford product C1 (526.3 mg, 53%, 64% bsrm), 93 mg digoxigenin was recovered. R_{f}=0.16 [60% EtOAc in hexanes]; mp 106-108° C.; [α]_{D}²⁰=−4.9 (c 3.4, CH₂Cl₂); IR (film) cm⁻¹ 3475 (w), 2937(m), 1732(s), 1696(s), 1447(m), 1266(m), 1077(s), 1021(s), 862(w), 733(s), 700(s); ¹H NMR (400 MHz, CDCl₃) δ 0.79 (s, 3H), 0.93 (s, 3H), 1.34 (d, 3H, J=6.8 Hz), 1.19-1.98 (m, 18H), 2.08-2.17 (m, 1H), 3.33 (dd, 1H, J=9.6, 5.6 Hz), 3.38 (dd, 1H, J=12.0, 3.6 Hz), 4.07 (s, 1H), 4.54 (q, 1H, J=6.8 Hz), 4.81, 4.91 (ABX, 2H, J_{AB}=18.0 Hz, J_{AX}=J_{BX}=1.6 Hz), 5.24 (d, 1H, J=3.6 Hz), 5.92 (s, 1H), 6.05 (d, 1H, J=10.0 Hz), 6.80 (dd, 1H, J=10.0, 3.2 Hz); ¹³C NMR (100 MHz, CDCl₃) δ 9.2, 15.5, 21.8, 23.9, 24.9, 26.7, 27.6, 30.3, 30.4, 32.2, 32.7, 33.4, 35.3, 36.9, 41.5, 45.8, 55.9, 70.6, 73.8, 74.1, 75.1, 86.0, 91.8, 117.7, 127.3, 144.7, 175.4, 175.5, 197.7; mass spectrum (ESI): m/e calcd for C₂₉H₄₁O₇⁺501.2852. found 501.2857 [0106].

Synthesis of C2:

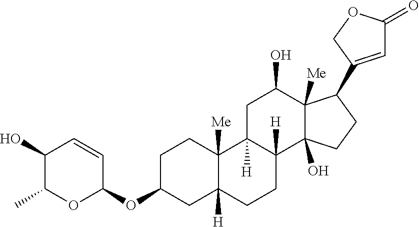

To a 0.1 ml CH₂Cl₂ solution of 16.4 mg C1 was added a solution of CeCl₃ in CH₃OH (0.4M, 0.1 ml), the reaction was then cooled to −78° C., and 1.4 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 80% EtOAc in hexanes to afford product C2 (15.1 mg, 91%). R_{f}=0.23 [80% EtOAc in hexanes]; mp 140-143° C.; [α]_{D}²⁰=+35.6 (c 1.8, CH₃OH); IR (film) cm⁻¹3411 (m), 2929(m), 1739(s), 1622(w), 1448(w), 1378(w), 1276(m), 1261(m), 1025(s), 1001(s), 764(s), 750(s); ¹H NMR (400 MHz, d₆-DMSO) δ 0.64 (s, 3H), 0.85 (s, 3H), 1.12 (d, 3H, J=4.4 Hz), 1.02-1.97 (m, 19H), 3.20-3.25 (m, 2H), 3.56-3.57 (m, 2H), 3.88 (s, 1H), 4.08 (s, 1H), 4.58 (d, 1H, J=5.2 Hz), 4.81, 4.90 (AB, 2H, J_{AB}=18.4 Hz), 4.94 (s, 1H), 5.03 (d, 1H, J=4.8 Hz), 5.60 (d, 1H, J=10.4 Hz), 5.78 (d, 1H, J=10.4 Hz), 5.81 (s, 1H); ¹³C NMR (100 MHz, d₆-DMSO) δ 10.1, 18.7, 22.0, 24.4, 25.5, 27.2, 27.5, 30.4, 30.9, 32.2, 32.5, 33.1, 35.4, 37.4, 41.2, 45.8, 56.4, 68.1, 68.9, 73.57, 73.63, 74.0, 85.0, 93.5, 116.5, 127.0, 134.8, 174.6, 177.6; mass spectrum (ESI): m/e calcd for C₂₉H₄₃O₇⁺503.3009. found 503.3014.

Synthesis of C3:

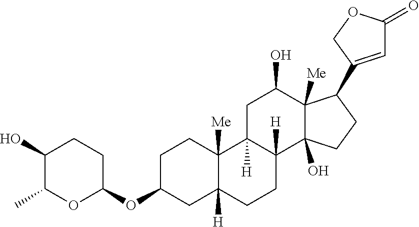

To a 1 ml NMM solution of 50 mg C2 was added 70 μl Et₃N and 217 mg NBSH at 0° C., the reaction was generally warmed up to room temperature and stirred for 24 hours. The reaction was quenched by saturated NaHCO₃ solution and extracted by EtOAc, dried over Na₂SO₄, and concentrated. The crude product was purified by flash chromatography with 80% EtOAc in hexanes to afford product C3 (40.5 mg, 80%). R_{f}=0.23 [80% EtOAc in hexanes]; mp 192-193° C.; [α]_{D}²⁰=+71.1 (c 1.0, CH₃OH); IR (film) cm⁻¹3436 (m), 2931(m), 1732(s), 1622(w), 1447(w), 1275(m), 1262(m), 1115(m), 1028(s), 988(s), 749(s); ¹H NMR (400 MHz, d₆-DMSO) δ 0.65 (s, 3H), 0.87 (s, 3H), 1.07 (d, 3H, J=6.0 Hz), 1.03-1.98 (m, 23H), 2.94-3.01 (m, 1H), 3.20-3.26 (m, 2H), 3.42-3.49 (m, 1H), 3.82 (s, 1H), 4.11 (s, 1H), 4.61 (d, 1H, J=5.2 Hz), 4.73 (s, 1H), 4.75 (d, 1H, J=6.0 Hz), 4.82, 4.91 (AB, 2H, J_{AB}=18.0 Hz), 5.82 (s, 1H); ¹³C NMR (100 MHz, CDCl₃-CD₃OD) δ 9.1, 17.9, 21.7, 23.8, 24.3, 26.8, 27.5, 29.9, 30.0, 30.3, 30.4, 32.3, 32.6, 33.0, 35.2, 36.9, 41.3, 45.8, 56.0, 69.8, 70.8, 72.0, 74.2, 74.8, 85.8, 94.1, 117.4, 175.9, 176.1; mass spectrum (ESI): m/e calcd for C₂₉H₄₅O₇⁺505.3165. found 505.3158.

Synthesis of C4:

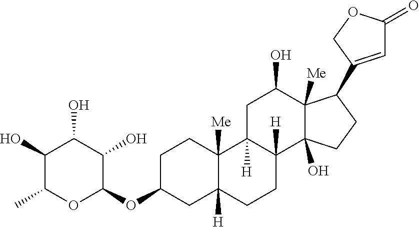

C2 (50 mg) was dissolved in t-BuOH/Acetone/CH₂Cl₂ (0.1 ml/0.1 ml/0.1 ml) and cooled to 0° C., NMO (50% w/v, 0.11 ml) was added, following by 0.5 mg OsO₄. The mixture was stirred at 0° C. for 24 hours. After the reaction was done, aqueous Na₂SO₃ solution was added, extracted by EtOAc, dried over Na₂SO₄, and concentrated. The crude product was purified by flash chromatography with 5% CH₃OH in CH₂Cl₂ to afford product C4 (51.6 mg, 96%). R_{f}=0.20 [10% MeOH in CHCl₃]; mp 234-236° C.; [α]_{D}²⁰=+60.1 (c 0.6, CH₃OH); IR (film) cm⁻¹ 3394 (m), 2931(m), 1731(s), 1625(w), 1448(m), 1381(m), 1275(m), 1046(s), 1021(s), 748(s); ¹H NMR (400 MHz, d₆-DMSO) δ 0.65 (s, 3H), 0.86 (s, 3H), 1.10 (d, 3H, J=6.4 Hz), 1.03-1.98 (m, 19H), 3.19-3.26 (m, 3H), 3.40-3.47 (m, 2H), 3.56 (s, 1H), 3.82 (s, 1H), 4.10 (s, 1H), 4.51 (d, 1H, J=5.6 Hz), 4.61 (s, 1H), 4.61 (d, 1H, J=5.2 Hz), 4.66 (d, 1H, J=4.4 Hz), 4.72 (d, 1H, J=5.6 Hz), 4.82, 4.91 (AB, 2H, J_{AB}=18.4 Hz), 5.82 (s, 1H); ¹³C NMR (100 MHz, d₆-DMSO) δ 10.1, 18.6, 22.0, 24.4(2), 27.2, 27.5, 30.4, 30.8, 32.2, 32.3, 33.1, 35.4, 37.4, 41.2, 45.9, 56.4, 69.3, 71.5, 71.6, 71.9, 72.7, 73.6, 74.0, 85.0, 98.9, 116.5, 174.6, 177.6; mass spectrum (ESI): m/e calcd for C₂₉H₄₅O₉⁺537.3064. found 537.3074.

Synthesis of C5:

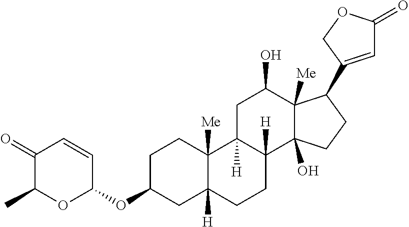

600 mg digoxigenin and 877 mg tert-butyl ((2S,6S)-6-methyl-5-oxo-5,6-dihydro-2H-pyran-2-yl) carbonate was dissolved in THF/CH₂Cl₂ (1:1, 15 ml/15 ml), and cooled to 0° C. 64 mg Pd₂(DBA)₃.CHCl₃ and 65 mg Ph₃P was dissolved in 8 ml CH₂Cl₂ and was added to the above mixture at 0° C. The reaction mixture was stirred at 0° C. for 24 hours, and then was quenched by saturated NaHCO₃ solution and extracted by EtOAc, dried over Na₂SO₄, and concentrated. The crude product was purified by flash chromatography with 20% EtOAc in hexanes to afford product C5 (487 mg, 63%, 69% bsrm), 60 mg digoxigenin was recovered. R_{f}=0.17 [60% EtOAc in hexanes]; mp 94-96° C.; [α]_{D}²⁰=+38.4 (c 2.8, CH₂Cl₂); IR (film) cm⁻¹3461 (w), 2936(m), 1733(s), 1697(s), 1626(w), 1448(m), 1265(m), 1078(s), 1020(s), 735(s); ¹H NMR (400 MHz, CDCl₃) δ 0.80 (s, 3H), 0.94 (s, 3H), 1.37 (d, 3H, J=6.4 Hz), 1.21-2.00 (m, 18H), 2.11-2.20 (m, 1H), 3.33 (dd, 1H, J=9.6, 6.4 Hz), 3.38 (dd, 1H, J=12.8, 4.4 Hz), 4.07 (s, 1H), 4.56 (q, 1H, J=6.4 Hz), 4.82, 4.90 (ABX, 2H, J_{AB}=18.0 Hz, J_{AX}=J_{BX}=1.6 Hz), 5.26 (d, 1H, J=3.6 Hz), 5.94 (s, 1H), 6.07 (d, 1H, J=10.0 Hz), 6.82 (dd, 1H, J=10.0, 3.6 Hz); ¹³C NMR (100 MHz, CDCl₃) δ 9.1, 15.5, 21.8, 23.9, 26.66, 26.73, 27.6, 30.52, 30.55, 30.6, 32.7, 33.5, 35.3, 36.7, 41.6, 45.8, 55.7, 70.7, 73.9, 74.2, 75.3, 86.1, 92.2, 118.0, 127.3, 144.5, 174.7, 174.9, 197.6; mass spectrum (ESI): m/e calcd for C₂₉H₄₁O₇⁺501.2852. found 501.2846.

Synthesis of C6:

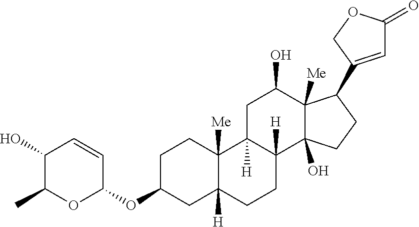

To a 0.9 ml CH₂Cl₂ solution of 210 mg C5 was added a solution of CeCl₃ in CH₃OH (0.4M, 0.9 ml), the reaction was then cooled to −78° C., and 18 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 60% EtOAc in hexanes to afford product C6 (183.6 mg, 87%). R_{f}=0.11 [60% EtOAc in hexanes]; mp 181-184° C.; [α]_{D}²⁰=+10.8 (c 1.4, CH₃OH); IR (film) cm⁻¹3436 (w), 2936(w), 1732(s), 1616(w), 1446(w), 1375(m), 1217(m), 1027(s); ¹H NMR (400 MHz, d₆-DMSO) δ 0.65 (s, 3H), 0.86 (s, 3H), 1.14 (d, 3H, J=6.4 Hz), 1.07-1.98 (m, 19H), 3.19-3.26 (m, 2H), 3.56-3.59 (m, 2H), 3.88 (s, 1H), 4.11 (s, 1H), 4.61 (d, 1H, J=5.2 Hz), 4.82, 4.91 (AB, 2H, J_{AB}=18.4 Hz), 4.96 (d, 1H, J=2.4 Hz), 5.06 (d, 1H, J=6.0 Hz), 5.61 (d, 1H, J=10.4 Hz), 5.79 (d, 1H, J=10.4 Hz), 5.82 (s, 1H); ¹³C NMR (100 MHz, d₆-DMSO) δ 10.1, 18.7, 22.0, 24.4, 26.9, 27.1, 27.5, 30.4, 31.0, 31.2, 32.3, 33.1, 35.3, 37.1, 41.2, 45.9, 56.4, 68.1, 68.8, 73.6, 73.8, 74.0, 85.0, 93.7, 116.5, 127.0, 134.8, 174.6, 177.6; mass spectrum (ESI): m/e calcd for C₂₉H₄₃O₇⁺503.3009. found 503.3015.

Synthesis of C7:

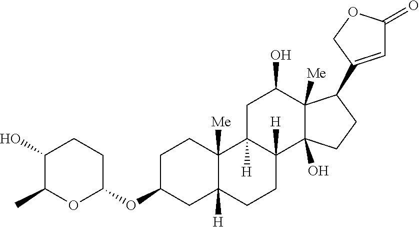

To a 1 ml NMM solution of 49.8 mg C6 was added 70 μl Et₃N and 217 mg NBSH at 0° C., the reaction was generally warmed up to room temperature and stirred for 24 hours. The reaction was quenched by saturated NaHCO₃ solution and extracted by EtOAc, dried over Na₂SO₄, and concentrated. The crude product was purified by flash chromatography with 70% EtOAc in hexanes to afford product C7 (45 mg, 90%). R_{f}=0.11 [60% EtOAc in hexanes]; mp 230-232° C.; [α]_{D}²⁰=−29.1 (c 0.5, CH₃OH); IR (film) cm⁻¹ 3448 (m), 2934(m), 1736(s), 1618(w), 1449(w), 1277(w), 1276(w), 1115(w), 1029(s); ¹H NMR (400 MHz, CD₃OD) δ 0.79 (s, 3H), 0.97 (s, 3H), 1.17 (d, 3H, J=6.4 Hz), 1.22-2.03 (m, 22H), 2.09-2.21 (m, 1H), 3.14 (q, 1H, J=7.2 Hz), 3.32-3.36 (m, 1H), 3.40 (dd, 1H, J=11.6, 4.4 Hz), 3.61-3.68 (m, 1H), 3.92 (s, 1H), 4.81 (s, 1H), 4.91, 4.98 (ABX, 2H, J_{AB}=18.4 Hz, J_{AX}=J_{BX}=1.6 Hz), 5.91 (s, 1H); ¹³C NMR (100 MHz, CD₃OD) δ 8.7, 17.1, 21.6, 23.1, 26.4, 26.6, 27.2, 29.7, 29.8, 30.2, 30.5, 32.3, 32.4, 35.1(2), 36.9, 41.0, 45.9, 56.1, 69.8, 71.5, 71.7, 74.3, 74.5, 85.6, 94.4, 116.5, 176.2, 177.3; mass spectrum (ESI): m/e calcd for C₂₉H₄₅O₇⁺505.3165. found 505.3173.

Synthesis of C8:

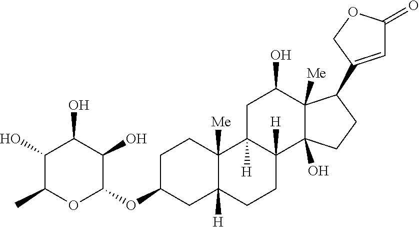

C6 (49.5 mg) was dissolved in t-BuOH/Acetone/CH₂Cl₂ (0.1 ml/0.1 ml/0.1 ml) and cooled to 0° C., NMO (50% w/v, 0.11 ml) was added, following by 0.5 mg OsO₄. The mixture was stirred at 0° C. for 24 hours. After the reaction was done, aqueous Na₂SO₃ solution was added, extracted by EtOAc, dried over Na₂SO₄, and concentrated. The crude product was purified by flash chromatography with 5% CH₃OH in CH₂Cl₂ to afford product C8 (26 mg, 49%). R_{f}=0.15 [10% MeOH in CHCl₃]; mp>250° C.; [α]_{D}²⁰=−20.7 (c 0.5, CH₃OH); IR (film) cm⁻¹ 3468 (w), 2990(w), 1738(s), 1623(w), 1447(w), 1366(m), 1275(s), 1261(s), 1015(m); ¹H NMR (400 MHz, CD₃OD) δ 0.78 (s, 3H), 0.96 (s, 3H), 1.23 (d, 3H, J=6.0 Hz), 1.17-2.03 (m, 18H), 2.11-2.16 (m, 1H), 3.31-3.41 (m, 3H), 3.61-3.70 (m, 2H), 3.75-3.76 (m, 1H), 3.94 (s, 1H), 4.76 (s, 1H), 4.90, 4.98 (AB, 2H, J_{AB}=18.4 Hz), 5.90 (s, 1H); ¹³C NMR (100 MHz, CD₃OD) δ 8.7, 16.8, 21.6, 23.1, 26.3(2), 26.6, 27.2, 29.6, 30.5, 32.4(2), 35.1, 37.0, 41.0, 45.8, 56.1, 68.8, 71.3, 71.7, 72.3, 72.9, 74.3, 74.4, 85.6, 98.7, 116.5, 176.2, 177.3; mass spectrum (ESI): m/e calcd for C₂₉H₄₅O₉ ⁺ 537,3064. found 537,3055.

### Example 2

### Synthesis of Protecting Group Deactivated Digoxin Monosaccharides

The protecting group deactivated Digoxin Monosaccharides (prodrug compounds) were synthesized using the procedure depicted in Scheme 2:

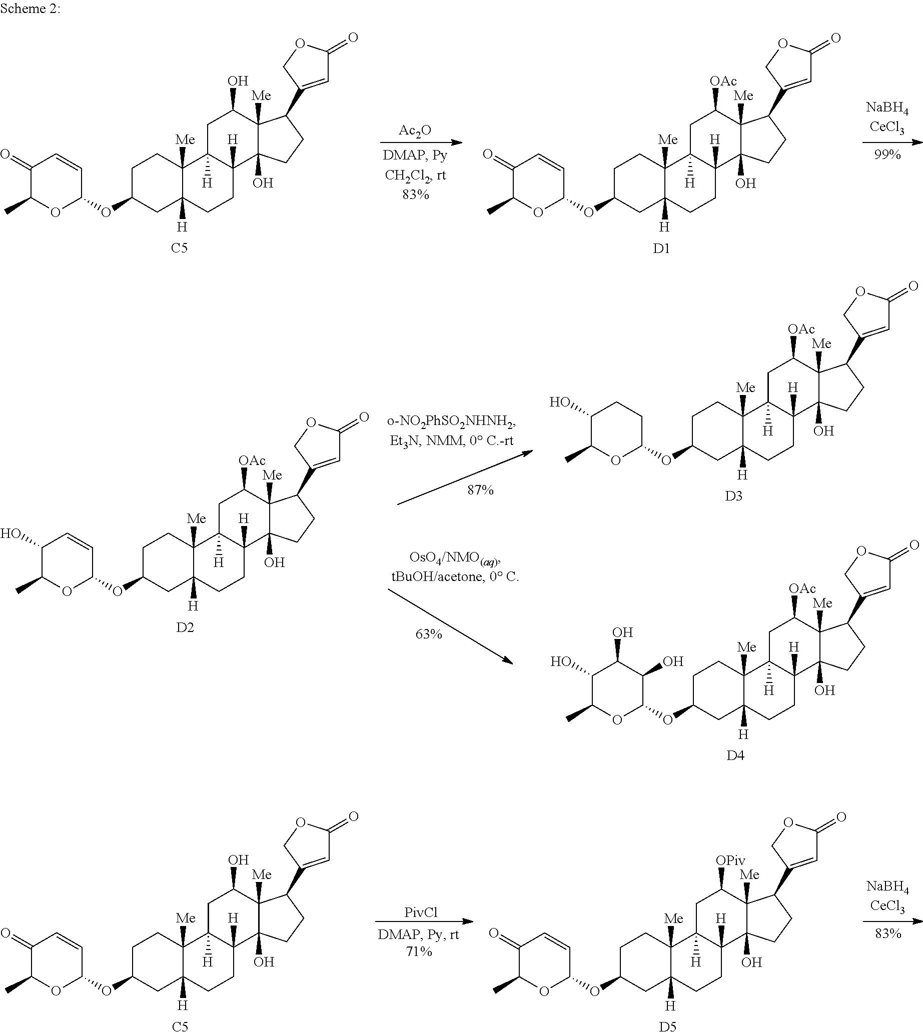

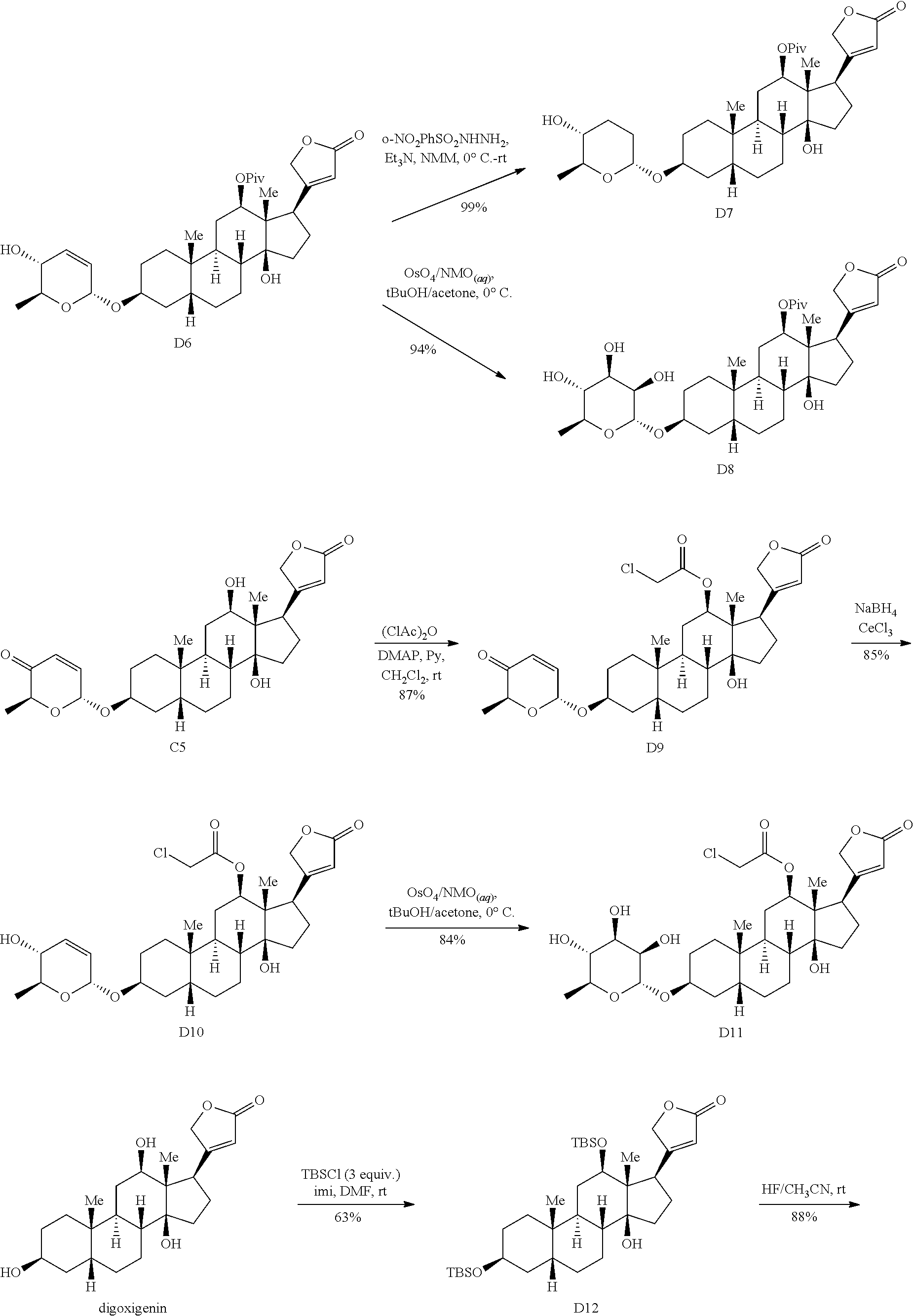

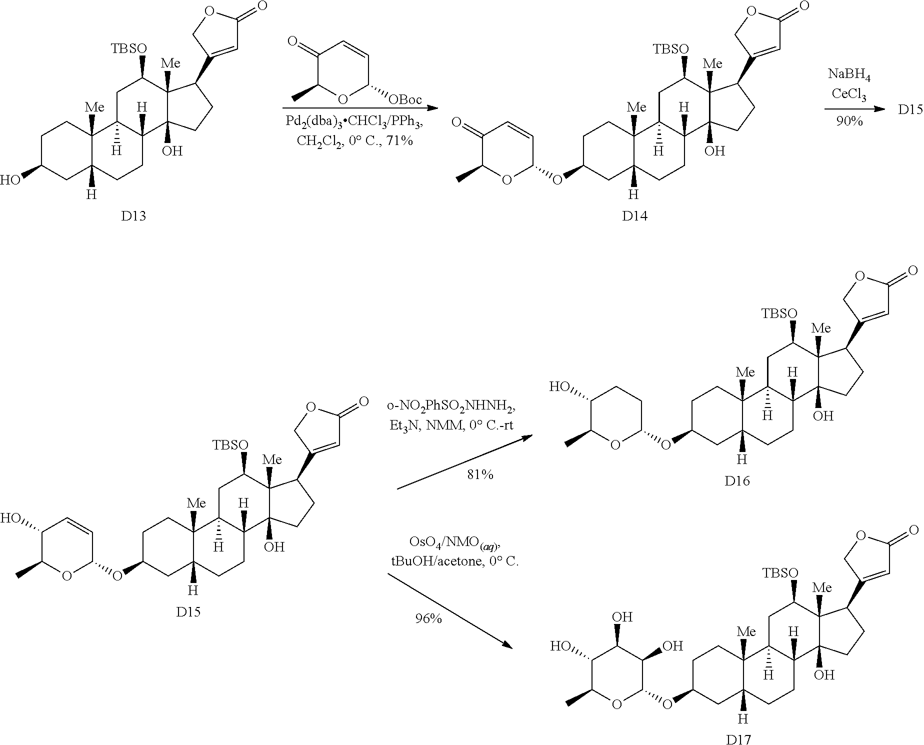

Synthesis of D1:

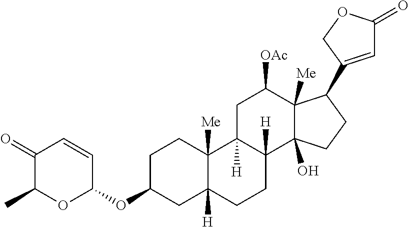

To a 0.22 ml CH₂Cl₂ solution of 5.5 mg C5 was added 10 μl pyridine, 10 μl acetic anhydride and 1.3 mg DMAP at 0° C. The reaction was stirred at 0° C. for 10 mins and then warmed up to room temperature and stirred for 18 hours. After the reaction was done, the mixture was quenched by saturated NaHCO₃ solution and extracted by EtOAc, dried over Na₂SO₄, and concentrated. The crude product was purified by flash chromatography with 40% EtOAc in hexanes to afford product D1 (5 mg, 83%). R_{f}=0.38 [60% EtOAc in hexanes]; mp 114-116° C.; [α]_{D}²⁰=+64.7 (c 2.0, CH₂Cl₂); IR (film) cm⁻¹3489 (w), 2936(m), 1731(s), 1698(s), 1628(w), 1448(w), 1373(m), 1240(s), 1155(w), 1078(m), 1022(s); ¹H NMR (400 MHz, CDCl₃) δ 0.81 (s, 3H), 0.86 (s, 3H), 1.27 (d, 3H, J=6.8 Hz), 1.17-1.96 (m, 18H), 2.02 (s, 3H), 2.06-2.09 (m, 1H), 2.34 (s, 1H), 2.81-2.84 (m, 1H), 3.99 (s, 1H), 4.47 (q, 1H, J=6.8 Hz), 4.55 (dd, 1H, J=11.2, 3.2 Hz), 4.72, 4.85 (AB, 2H, J_{AB}=18.4 Hz), 5.19 (d, 1H, J=3.2 Hz), 5.76 (s, 1H), 5.97 (d, 1H, J=10.4 Hz), 6.75 (dd, 1H, J=10.4, 3.2 Hz); ¹³C NMR (100 MHz, CDCl₃) δ 10.6, 15.4, 21.5, 21.7, 23.7, 26.60, 26.65, 27.4, 30.5, 30.6, 32.4, 33.1, 35.4, 36.7, 41.3, 46.1, 54.3, 70.5, 73.7, 74.3, 77.1, 77.6, 85.7, 92.1, 117.9, 127.1, 144.7, 171.1, 174.4, 174.9, 197.6; mass spectrum (ESI): m/e calcd for C₃₁H₄₃O₈⁺543.2958. found 543.2949.

Synthesis of D2:

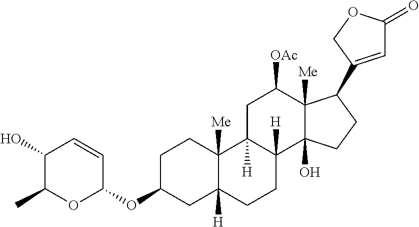

To a 1.2 ml CH₂Cl₂ solution of 271.3 mg D1 was added a solution of CeCl₃ in CH₃OH (0.4M, 1.2 ml), the reaction was then cooled to −78° C., and 21 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 5% MeOH in CHCl₃ to afford product D2 (269.6 mg, 99%). R_{f}=0.24 [5% MeOH in CHCl₃]; mp 120-122° C.; [α]_{D}²⁰=+30.2 (c 2.4, CH₂Cl₂); IR (film) cm⁻¹ 3450 (m), 2934(m), 1732(s), 1626(w), 1448(w), 1379(m), 1242(s), 1074(w), 1026(s); ¹H NMR (400 MHz, CDCl₃) δ 0.88 (s, 3H), 0.92 (s, 3H), 1.28 (d, 3H, J=6.4 Hz), 1.18-2.03 (m, 18H), 2.09 (s, 3H), 2.12-2.19 (m, 1H), 2.87-2.91 (m, 1H), 3.72 (dq, 1H, J=8.8, 6.4 Hz), 3.83 (d, 1H, J=8.4 Hz), 3.97 (s, 1H), 4.62 (dd, 1H, J=12.0, 4.0 Hz), 4.77, 4.88 (ABX, 2H, J_{AB}=18.0 Hz, J_{AX}=J_{BX}=1.6 Hz), 5.00 (d, 1H, J=0.8 Hz), 5.71 (dt, 1H, J=10.0, 2.4 Hz), 5.83 (s, 1H), 5.90 (d, 1H, J=10.0 Hz); ¹³C NMR (100 MHz, CDCl₃) δ 10.6, 18.2, 21.5, 21.8, 23.8, 26.66, 26.70, 26.9, 27.4, 30.5, 30.9, 32.6, 33.4, 35.4, 36.7, 41.6, 46.2, 54.2, 68.2, 70.0, 73.6, 73.7, 77.7, 86.0, 93.5, 118.2, 127.7, 133.2, 171.1, 173.8, 174.7; mass spectrum (ESI): m/e calcd for C₃₁H₄₅O₈⁺545.3114. found 545.3102.

Synthesis of D3:

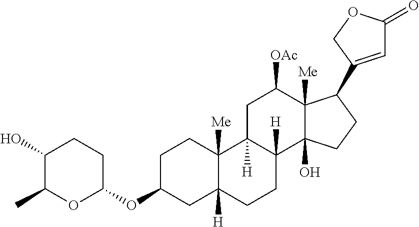

To a 1 ml NMM solution of 54 mg D2 was added 70 μl Et₃N and 217 mg NBSH at 0° C., the reaction was generally warmed up to room temperature and stirred for 24 hours. The reaction was quenched by saturated NaHCO₃ solution and extracted by EtOAc, dried over Na₂SO₄, and concentrated. The crude product was purified by flash chromatography with 5% MeOH in CHCl₃ to afford product D3 (47.4 mg, 87%). R_{f}=0.24 [5% MeOH in CHCl₃]; mp 156-160° C.; [α]_{D}²⁰=+2.8 (c 1.3, CH₂Cl₂); IR (film) cm⁻¹ 3459 (m), 2935(m), 1735(s), 1626(w), 1448(w), 1340(w), 1243(m), 1159(w), 1048(s); ¹H NMR (400 MHz, CDCl₃) δ 0.88 (s, 3H), 0.93 (s, 3H), 1.22 (d, 3H, J=6.4 Hz), 1.19-2.04 (m, 22H), 2.09 (s, 3H), 2.12-2.20 (m, 1H), 2.87-2.91 (m, 1H), 3.23-3.28 (m, 1H), 3.62 (dq, 1H, J=8.8, 6.4 Hz), 3.91 (s, 1H), 4.62 (dd, 1H, J=12.0, 4.0 Hz), 4.77, 4.88 (AB, 2H, J_{AB}=18.0 Hz), 4.81 (s, 1H), 5.84 (s, 1H); ¹³C NMR (100 MHz, CDCl₃) δ 10.6, 18.1, 21.5, 21.9, 23.8, 26.69, 26.75, 26.8, 27.4, 27.9, 30.0, 30.5, 30.7, 32.5, 33.4, 35.4, 36.7, 41.6, 46.1, 54.2, 69.8, 70.9, 72.5, 73.6, 77.7, 86.1, 94.3, 118.2, 171.1, 173.7, 174.6; mass spectrum (ESI): m/e calcd for C₃₁H₄₇O₈⁺547.3271. found 547.3275.

Synthesis of D4:

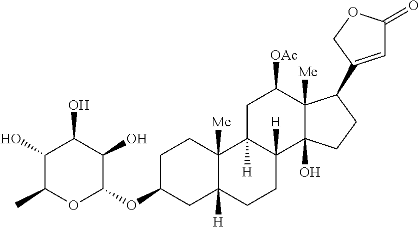

D2 (55 mg) was dissolved in t-BuOH/Acetone (0.1 ml/0.1 ml) and cooled to 0° C., NMO (50% w/v, 0.11 ml) was added, following by 0.5 mg OsO₄. The mixture was stirred at 0° C. for 24 hours. After the reaction was done, aqueous Na₂SO₃ solution was added, extracted by EtOAc, dried over Na₂SO₄, and concentrated. The crude product was purified by flash chromatography with 10% CH₃OH in CH₂Cl₂ to afford product D4 (36.4 mg, 63%). R_{f}=0.15 [10% MeOH in CHCl₃]; mp 235-238° C.; [α]_{D}²⁰=+3.5 (c 0.5, CH₃OH); IR (film) cm⁻¹ 3433 (m), 2929(m), 1731(s), 1626(w), 1448(m), 1376(m), 1259(s), 1045(s), 1021(s); ¹H NMR (400 MHz, CD₃OD) δ 0.91 (s, 3H), 0.96 (s, 3H), 1.24 (d, 3H, J=6.4 Hz), 1.26-2.08 (m, 18H), 2.10 (s, 3H), 2.11-2.20 (m, 1H), 2.98 (dd, 1H, J=9.6, 6.4 Hz), 3.37 (dd, 1H, J=9.6 Hz), 3.62-3.70 (m, 2H), 3.76 (dd, 1H, J=3.2, 1.6 Hz), 3.96 (s, 1H), 4.65 (dd, 1H, J=12.0, 4.0 Hz), 4.77 (s, 1H), 4.90, 4.99 (ABX, 2H, J_{AB}=18.4 Hz, J_{AX}=J_{BX}=1.6 Hz), 5.89 (s, 1H); ¹³C NMR (100 MHz, CD₃OD) δ 9.8, 16.8, 20.0, 21.5, 23.0, 26.2, 26.3, 26.6, 27.2, 29.5, 30.4, 32.2, 32.3, 35.1, 37.0, 41.0, 46.1, 54.3, 68.9, 71.3, 71.7, 72.2, 72.9, 74.1, 77.7, 85.4, 98.6, 116.9, 171.6, 175.8, 176.1; mass spectrum (ESI): m/e calcd for C₃₁H₄₇O₁₀⁺579.3169. found 579.3161.

Synthesis of D5:

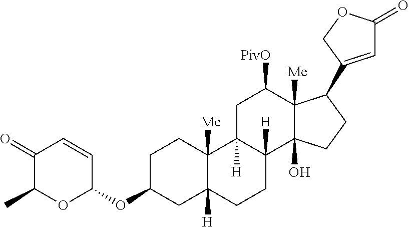

To a 0.5 ml pyridine solution of 20 mg C5 was added 50 μl pivaloyl chloride and 5 mg DMAP at room temperature. The reaction was stirred at room temperature for 20 hours. After the reaction was done, the mixture was quenched by saturated NaHCO₃ solution and extracted by EtOAc, dried over Na₂SO₄, and concentrated. The crude product was purified by flash chromatography with 40% EtOAc in hexanes to afford product D5 (16.6 mg, 71%). R_{f}=0.16 [40% EtOAc in hexanes]; mp 126-128° C.; [α]_{D}²⁰=+57.8 (c 0.8, CH₂Cl₂); IR (film) cm⁻¹ 3510 (w), 2937(m), 1741(s), 1448(w), 1366(m), 1283(w), 1229(w), 1163(m), 1079(w), 1031(m); ¹H NMR (400 MHz, CDCl₃) δ 0.92 (s, 3H), 0.94 (s, 3H), 1.23 (s, 9H), 1.36 (d, 3H, J=6.8 Hz), 1.20-2.08 (m, 18H), 2.16-2.24 (m, 1H), 2.93 (dd, 1H, J=9.6, 6.0 Hz), 4.07 (s, 1H), 4.53-4.59 (m, 2H), 4.80, 4.92 (ABX, 2H, J_{AB}=18.0 Hz, J_{AX}=J_{BX}=1.6 Hz), 5.26 (d, 1H, J=3.6 Hz), 5.84 (s, 1H), 6.07 (d, 1H, J=10.4 Hz), 6.81 (dd, 1H, J=10.4, 3.6 Hz); ¹³C NMR (100 MHz, CDCl₃) δ 10.6, 15.5, 21.7, 23.8, 26.3, 26.6, 26.7, 27.3, 27.5, 30.5, 30.7, 32.5, 33.5, 35.4, 36.7, 39.3, 41.5, 46.0, 54.5, 70.7, 73.6, 74.4, 76.6, 86.1, 92.3, 118.4, 127.3, 144.5, 173.8, 174.5, 178.4, 197.6; mass spectrum (ESI): m/e calcd for C₃₄H₄₉O₈⁺585.3427. found 585.3422.

Synthesis of D6:

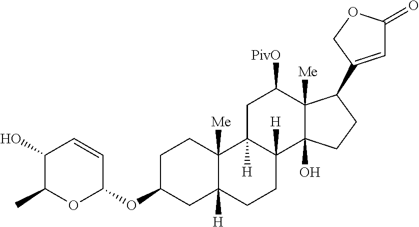

To a 0.2 ml CH₂Cl₂ solution of 41.4 mg D5 was added a solution of CeCl₃ in CH₃OH (0.4M, 0.2 ml), the reaction was then cooled to −78° C., and 3 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 50% EtOAc in hexanes to afford product D6 (34.3 mg, 83%). R_{f}=0.19 [50% EtOAc in hexanes]; mp 146-148° C.; [α]_{D}²⁰=+21.6 (c 0.8, CH₂Cl₂); IR (film) cm⁻¹3451 (w), 2928(m), 1726(s), 1622(w), 1448(w), 1378(m), 1283(m), 1163(s), 1032(s); ¹H NMR (400 MHz, CDCl₃) δ 0.90 (s, 3H), 0.93 (s, 3H), 1.22 (s, 9H), 1.28 (d, 3H, J=6.4 Hz), 1.17-2.06 (m, 18H), 2.13-2.22 (m, 1H), 2.92 (dd, 1H, J=9.6, 6.0 Hz), 3.73 (qt, 1H, J=8.8, 6.4 Hz), 3.83 (d, 1H, J=8.4 Hz), 3.97 (s, 1H), 4.56 (dd, 1H, J=12.0, 4.0 Hz), 4.79, 4.93 (AB, 2H, J_{AB}=18.0 Hz), 5.00 (s, 1H), 5.71 (dt, 1H, J=10.0, 2.4 Hz), 5.83 (s, 1H), 5.90 (d, 1H, J=10.0 Hz); ¹³C NMR (100 MHz, CDCl₃) δ 10.7, 18.2, 21.7, 23.8, 26.3, 26.7, 27.0, 27.3, 27.5, 30.5, 31.0, 32.5, 33.4, 35.4, 36.7, 39.3, 41.4, 46.1, 54.6, 68.2, 70.0, 73.7, 73.8, 76.7, 86.1, 93.5, 118.3, 127.7, 133.2, 174.2, 174.7, 178.5; mass spectrum (ESI): m/e calcd for C₃₄H₅₁O₈⁺587.3584. found 587.3583.

Synthesis of D7:

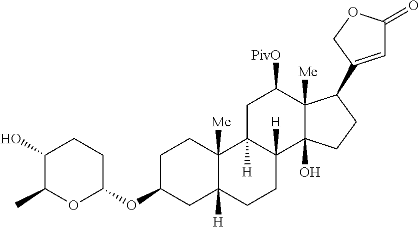

To a 0.5 ml NMM solution of 30 mg D6 was added 70 μl Et₃N and 111 mg NBSH at 0° C., the reaction was generally warmed up to room temperature and stirred for 24 hours. The reaction was quenched by saturated NaHCO₃ solution and extracted by EtOAc, dried over Na₂SO₄, and concentrated. The crude product was purified by flash chromatography with 50% EtOAc in hexanes to afford product D7 (30 mg, 99%). R_{f}=0.19 [50% EtOAc in hexanes]; mp 145-146° C.; [α]_{D}²⁰=−0.7 (c 0.4, CH₂Cl₂); IR (film) cm⁻¹ 3459 (w), 2935(m), 1740(s), 1449(w), 1366(m), 1280(m), 1163(m), 1030(m); ¹H NMR (400 MHz, CDCl₃) δ 0.85 (s, 3H), 0.88 (s, 3H), 1.17 (s, 9H), 1.13-2.02 (m, 25H), 2.09-2.17 (m, 1H), 2.87 (dd, 1H, J=9.6, 6.0 Hz), 3.18-3.22 (m, 1H), 3.56 (dq, 1H, J=9.2, 6.4 Hz), 3.85 (s, 1H), 4.51 (dd, 1H, J=11.6, 4.4 Hz), 4.75 (s, 1H), 4.73, 4.86 (AB, 2H, J_{AB}=18.0 Hz), 5.78 (s, 1H); ¹³C NMR (100 MHz, CDCl₃) δ 10.6, 18.1, 21.8, 23.9, 26.4, 26.7, 26.9, 27.3, 27.5, 27.9, 30.1, 30.5, 30.6, 32.5, 33.4, 35.4, 36.7, 39.3, 41.5, 46.1, 54.5, 69.8, 71.1, 72.6, 73.6, 76.9, 86.1, 94.4, 118.4, 173.9, 174.6, 178.4; mass spectrum (ESI): m/e calcd for C₃₄H₅₃O₈⁺589.3740. found 589.3737.

Synthesis of D8:

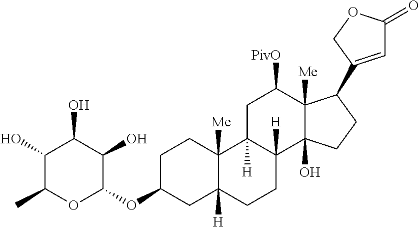

D6 (34 mg) was dissolved in t-BuOH/Acetone (0.25 ml/0.25 ml) and cooled to 0° C., NMO (50% w/v, 0.1 ml) was added, following by 0.3 mg OsO₄. The mixture was stirred at 0° C. for 18 hours. After the reaction was done, aqueous Na₂SO₃ solution was added, extracted by EtOAc, dried over Na₂SO₄, and concentrated. The crude product was purified by flash chromatography with 10% CH₃OH in CH₂Cl₂ to afford product D8 (29.8 mg, 94%). R_{f}=0.15 [10% MeOH in CHCl₃]; mp 220-223° C.; [α]_{D}²⁰=+2.5 (c 0.6, CH₃OH); IR (film) cm⁻¹ 3437 (m), 2933(m), 1738(s), 1622(w), 1448(m), 1396(m), 1276(s), 1229(m), 1162(m), 1045(s); ¹H NMR (400 MHz, CDCl₃) δ 0.91 (s, 3H), 0.93 (s, 3H), 1.23 (s, 9H), 1.29 (d, 3H, J=6.4 Hz), 1.18-2.06 (m, 18H), 2.15-2.23 (m, 1H), 2.52 (s, 1H), 2.75 (s, 1H), 2.92 (dd, 1H, J=9.6, 5.6 Hz), 3.45 (dd, 1H, J=9.2 Hz), 3.67-3.74 (m, 1H), 3.81 (d, 1H, J=8.0 Hz), 3.90 (s, 1H), 3.95 (s, 1H), 4.56 (dd, 1H, J=11.6, 4.0 Hz), 4.86 (s, 1H), 4.79, 4.92 (AB, 2H, J_{AB}=18.4 Hz), 5.84 (s, 1H); ¹³C NMR (100 MHz, CDCl₃) δ 10.6, 17.7, 21.8, 23.8, 26.3, 26.6, 26.7, 27.3, 27.5, 29.7, 30.5, 32.5, 33.5, 35.4, 36.7, 39.3, 41.5, 46.0, 54.5, 68.1, 71.8, 72.0, 72.1, 73.6, 74.0, 76.6, 86.1, 97.8, 118.4, 173.8, 174.5, 178.4; mass spectrum (ESI): m/e calcd for C₃₄H₅₃O₁₀⁺621.3639. found 621.3639.

Synthesis of D9:

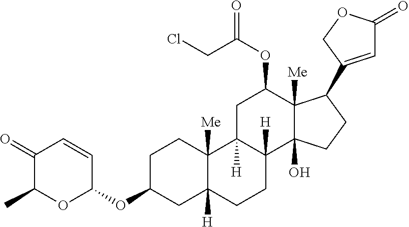

To a 1 ml CH₂Cl₂ solution of 20 mg C5 was added 50 μl pyridine, 68.4 mg chloroacetic anhydride and 5 mg DMAP at 0° C. The reaction was stirred at 0° C. for 10 mins and then warmed up to room temperature and stirred for 24 hours. After the reaction was done, the mixture was quenched by saturated NaHCO₃ solution and extracted by EtOAc, dried over Na₂SO₄, and concentrated. The crude product was purified by flash chromatography with 30% EtOAc in hexanes to afford product D9 (20 mg, 87%). R_{f}=0.39 [60% EtOAc in hexanes]; mp 98-99° C.; [α]_{D}²⁰=+53.8 (c 0.7, CH₂Cl₂); IR (film) cm⁻¹ 3503 (w), 2937(m), 1742(s), 1699(m), 1628(w), 1448(w), 1373(w), 1288(w), 1203(w), 1079(w), 1031(m); ¹H NMR (400 MHz, CDCl₃) δ 0.92 (s, 3H), 0.94 (s, 3H), 1.36 (d, 3H, J=6.8 Hz), 1.23-2.02 (m, 18H), 2.15-2.25 (m, 1H), 2.91 (dd, 1H, J=9.2, 6.0 Hz), 4.04-4.12 (m, 3H), 4.55 (q, 1H, J=6.8 Hz), 4.68 (dd, 1H, J=12.0, 4.0 Hz), 4.80, 4.89 (AB, 2H, J_{AB}=18.0 Hz), 5.26 (d, 1H, J=3.6 Hz), 5.91 (s, 1H), 6.07 (d, 1H, J=10.0 Hz), 6.81 (dd, 1H, J=10.0, 3.2 Hz); ¹³C NMR (100 MHz, CDCl₃) δ 10.6, 15.5, 21.8, 23.8, 26.5, 26.6, 26.7, 27.5, 30.5, 30.7, 32.5, 33.4, 35.4, 36.6, 41.2, 41.5, 46.0, 54.3, 70.7, 73.6, 74.2, 79.8, 86.0, 92.2, 118.5, 127.3, 144.5, 167.4, 173.4, 174.6, 197.6; mass spectrum (ESI): m/e calcd for C₃₁H₄₂ClO₈⁺577.2568. found 577.2578.

Synthesis of D10:

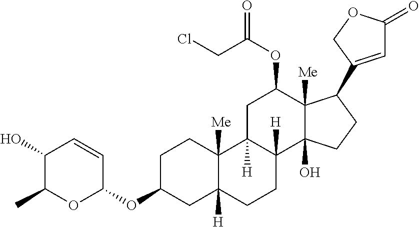

To a 0.15 ml CH₂Cl₂ solution of 21.3 mg D9 was added a solution of CeCl₃ in CH₃OH (0.4M, 0.15 ml), the reaction was then cooled to −78° C., and 2 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 60% EtOAc in hexanes to afford product D10 (18.2 mg, 85%). R_{f}=0.30 [60% EtOAc in hexanes]; mp 178-180° C.; [α]_{D}²⁰=+22.2 (c 1.4, CH₂Cl₂); IR (film) cm⁻¹3459 (w), 2931(m), 1737(s), 1625 (w), 1448(w), 1379(w), 1277(m), 1186(m), 1033(s); ¹H NMR (400 MHz, CDCl₃) δ 0.92 (s, 3H), 0.94 (s, 3H), 1.29 (d, 3H, J=6.4 Hz), 1.20-2.04 (m, 18H), 2.15-2.23 (m, 1H), 2.89-2.93 (m, 1H), 3.69-3.76 (m, 1H), 3.84 (dd, 1H, J=8.0, 8.0 Hz), 3.98 (s, 1H), 4.03-4.11(m, 2H), 4.67 (dd, 1H, J=12.0, 4.0 Hz), 4.80, 4.89 (AB, 2H, J_{AB}=18.0 Hz), 5.01 (s, 1H), 5.73 (d, 1H, J=10.0), 5.91 (s, 1H), 5.90-5.92 (m, 1H); ¹³C NMR (100 MHz, CDCl₃) δ 10.6, 18.2, 21.8, 23.8, 26.5, 26.6, 26.9, 27.4, 30.5, 30.9, 32.5, 33.4, 35.4, 36.6, 41.3, 41.5, 46.0, 54.3, 68.2, 70.0, 73.7(2), 79.9, 86.1, 93.5, 118.5, 127.8, 133.2, 167.4, 173.4, 174.6; mass spectrum (ESI): m/e calcd for C₃H₄₄ClO₈⁺579.2725. found 579.2727.

Synthesis of D11:

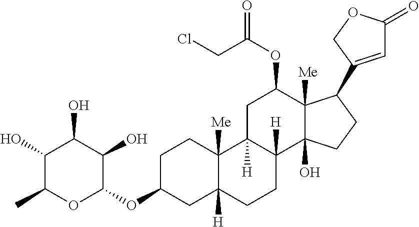

D10 (50 mg) was dissolved in t-BuOH/Acetone (0.4 ml/0.4 ml) and cooled to 0° C., NMO (50% w/v, 0.1 ml) was added, following by 0.5 mg OsO₄. The mixture was stirred at 0° C. for 18 hours. After the reaction was done, aqueous Na₂SO₃ solution was added, extracted by EtOAc, dried over Na₂SO₄, and concentrated. The crude product was purified by flash chromatography with 5% CH₃OH in CH₂Cl₂ to afford product D11 (44.5 mg, 84%). R_{f}=0.25 [5% MeOH in EtOAc]; mp 206-208° C.; [α]_{D}²⁰=−1.4 (c 0.6, CH₃OH); IR (film) cm⁻¹ 3435 (m), 2931(m), 1737(s), 1624(w), 1448(m), 1373(m), 1262(m), 1217(m), 1045(m); ¹H NMR (400 MHz, CDCl₃) δ 0.92 (s, 3H), 0.94 (s, 3H), 1.30 (d, 3H, J=6.4 Hz), 1.18-2.03 (m, 18H), 2.15-2.22 (m, 1H), 2.91 (dd, 1H, J=9.6, 5.6 Hz), 3.45 (dd, 1H, J=9.6, 9.6 Hz), 3.69-3.74 (m, 1H), 3.80 (dd, 1H, J=9.6, 3.6 Hz), 3.90 (m, 1H), 3.97 (s, 1H), 4.04-4.12 (m, 2H), 4.67 (dd, 1H, J=12.0, 4.0 Hz), 4.86 (s, 1H), 4.80, 4.89 (ABX, 2H, J_{AB}=18.0 Hz, J_{AX}=J_{BX}=1.2 Hz), 5.91 (s, 1H); ¹³C NMR (100 MHz, CD₃OD) δ 10.5, 17.7, 21.8, 23.8, 26.5, 26.6, 27.4, 29.6, 29.9, 30.5, 32.5, 33.4, 35.4, 36.6, 41.2, 41.5, 46.0, 54.3, 68.1, 71.75, 71.81, 72.1, 73.6, 74.0, 79.8, 86.0, 97.7, 118.6, 167.4, 173.3, 174.5; mass spectrum (ESI): m/e calcd for C₃₁H₄₆ClO₁₀⁺613.2780. found 613.2772.

Synthesis of D12:

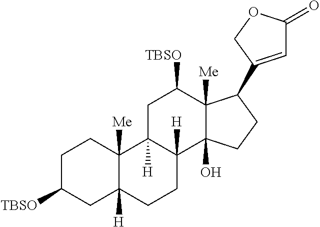

To a DMF (5 ml) solution of 390.5 mg digoxigenin and 272 mg imidazole at 0° C. was added 24.5 mg DMAP and 452 mg TBSCl. Then the reaction was stirred at 0° C. for 5 mins, then warmed up to room temperature and stirred for 15 hours. After the reaction was done, the mixture was quenched by H₂O and extracted by EtOAc threes times, then washed by H₂O five times, the organic phase was then dried over Na₂SO₄, and concentrated. The crude product was purified by flash chromatography with 20% EtOAc in hexanes to afford product D12 (390 mg, 63%). R_{f}=0.79 [80% EtOAc in hexanes]; mp 228-229° C.; [α]_{D}²⁰=+20.8 (c 0.5, CH₂Cl₂); IR (film) cm⁻¹3488 (w), 2929(m), 2858(m), 1742(s), 1622(w), 1471(w), 1377(w), 1257(m), 1157(w), 1053(s); ¹H NMR (400 MHz, CDCl₃) δ 0.00 (s, 6H), 0.03 (s, 3H), 0.05 (s, 3H), 0.78 (s, 3H), 0.86 (s, 9H), 0.89 (s, 9H), 0.91 (s, 3H), 1.16-1.94 (m, 18H), 2.02-2.12 (m, 1H), 3.17 (dd, 1H, J=10.0, 5.2 Hz), 3.32 (dd, 1H, J=11.2, 4.0 Hz), 4.02 (s, 1H), 4.77, 4.89 (AB, 2H, J_{AB}=18.4 Hz), 5.86 (s, 1H); ¹³C NMR (100 MHz, CDCl₃) δ −4.7, −4.6, −4.4, −3.6, 9.8, 18.2, 18.3, 21.9, 23.9, 26.0, 26.1, 26.8, 27.3, 28.6, 30.1, 30.3, 32.5, 33.4, 34.3, 35.2, 36.2, 41.6, 45.7, 56.8, 67.2, 74.0, 76.0, 86.2, 117.7, 175.1, 175.4; mass spectrum (ESI): m/e calcd for C₃₅H₆₃O₅Si₂⁺619.4214. found 619.4230.

Synthesis of D13:

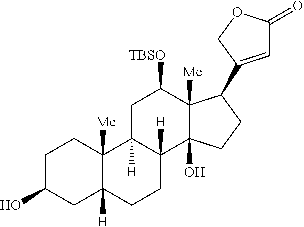

5% HF in CH₃CN (7.4 ml) was added to TBS ether D12 (285 mg) at room temperature and stirred for 24 hours. After the reaction was done, saturated NaHCO₃ solution was added and extracted with EtOAc, the organic phase was combined and dried over Na₂SO₄. The crude product was purified by flash chromatography with 40% EtOAc in hexanes to afford product D13 (205 mg, 88%). R_{f}=0.12 [40% EtOAc in hexanes]; mp>250° C.; [α]_{D}²⁰=+23.3 (c 0.3, MeOH); IR (film) cm⁻¹ 3428 (w), 2925(m), 2852(m), 1729(s), 1615(w), 1443(w), 1365(m), 1260(s), 1229(m), 1089(w), 1026(m); ¹H NMR (400 MHz, CDCl₃) δ 0.00 (s, 3H), 0.01 (s, 3H), 0.74 (s, 3H), 0.85 (s, 9H), 0.90 (s, 3H), 1.14-1.90 (m, 18H), 1.99-2.06 (m, 1H), 3.13 (dd, 1H, J=9.6, 4.8 Hz), 3.28 (dd, 1H, J=11.6, 4.0 Hz), 4.07 (s, 1H), 4.73, 4.82 (AB, 2H, J_{AB}=18.0 Hz), 5.83 (s, 1H); ¹³C NMR (100 MHz, CDCl₃) δ −4.4, −3.5, 9.8, 18.3, 21.8, 23.8, 26.1, 26.5, 27.3, 27.9, 30.0, 30.2, 32.3, 33.4, 33.5, 35.4, 36.1, 41.6, 45.6, 56.7, 66.9, 73.9, 75.9, 86.2, 117.9, 174.9, 175.0; mass spectrum (ESI): m/e calcd for C₂₉H₄₉O₅Si⁺505.3349. found 505.3337.

Synthesis of D14:

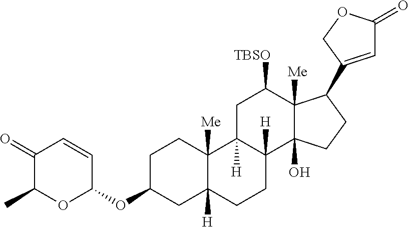

200 mg D13 and 228 mg tert-butyl ((2S,6S)-6-methyl-5-oxo-5,6-dihydro-2H-pyran-2-yl) carbonate was dissolved in THF/CH₂Cl₂ (1:2, 1.6 ml/3.2 ml), and cooled to 0° C. 20.7 mg Pd₂(DBA)₃.CHCl₃ and 21 mg Ph₃P was dissolved in 1 ml CH₂Cl₂ and was added to the above mixture at 0° C. The reaction mixture was stirred at 0° C. for 24 hours, and then was quenched by saturated NaHCO₃ solution and extracted by EtOAc, dried over Na₂SO₄, and concentrated. The crude product was purified by flash chromatography with 40% EtOAc in hexanes to afford product D14 (175 mg, 71%). R_{f}⁼0.21 [40% EtOAc in hexanes]; mp 220-222° C.; [α]_{D}²⁰=+47.9 (c 2.0, CH₂Cl₂); IR (film) cm⁻¹ 3503 (w), 2931(m), 2858(w), 1743(s), 1699(m), 1623(w), 1462(w), 1363(w), 1253(m), 1154(w), 1105(m), 1078(s), 1029(s); ¹H NMR (400 MHz, CDCl₃) δ 0.05 (s, 3H), 0.07 (s, 3H), 0.79 (s, 3H), 0.90 (s, 9H), 0.93 (s, 3H), 1.36 (d, 3H, J=6.8 Hz), 1.17-1.96 (m, 18H), 2.04-2.16 (m, 1H), 3.18 (dd, 1H, J=9.6, 4.8 Hz), 3.34 (dd, 1H, J=11.2, 4.0 Hz), 4.06 (s, 1H), 4.55 (q, 1H, J=6.8 Hz), 4.78, 4.88 (ABX, 2H, J_{AB}=18.0 Hz, J_{AX}=J_{BX}=1.6 Hz), 5.25 (d, 1H, J=3.6 Hz), 5.88 (s, 1H), 6.06 (d, 1H, J=10.0 Hz), 6.81 (dd, 1H, J=10.0, 3.6 Hz); ¹³C NMR (100 MHz, CDCl₃) δ −4.4, −3.5, 9.8, 15.5, 18.2, 21.8, 23.8, 26.1, 26.5, 26.6, 27.3, 30.2, 30.7, 32.5, 33.4, 35.2, 36.7, 41.6, 45.6, 56.8, 70.7, 73.9, 74.3, 75.9, 86.2, 92.3, 117.8, 127.3, 144.5, 174.9, 175.1, 197.5; mass spectrum (ESI): m/e calcd for C₂₉H₄₁O₂⁺615.3717. found 615.3700.

Synthesis of D15:

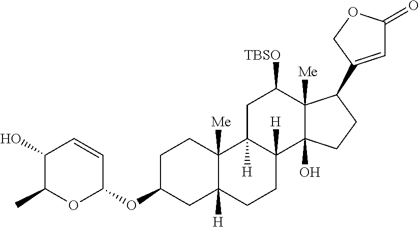

To a 0.3 ml CH₂Cl₂ solution of 61.5 mg D14 was added a solution of CeCl₃ in CH₃OH (0.4M, 0.3 ml), the reaction was then cooled to −78° C., and 4.2 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 40% EtOAc in hexanes to afford product D15 (55.5 mg, 90%). R_{f}=0.13 [40% EtOAc in hexanes]; mp 204-206° C.; [α]_{D}²⁰=+8.4 (c 2.3, CH₂Cl₂); IR (film) cm⁻¹3460 (w), 2928(m), 2856(m), 1729(s), 1621(w), 1461(w), 1382(w), 1252(m), 1094(m), 1033(s), 1000(s), 881(s), 833(s), 74(s), 735(s); ¹H NMR (500 MHz, CDCl₃) δ 0.05 (s, 3H), 0.06 (s, 3H), 0.78 (s, 3H), 0.90 (s, 9H), 0.91 (s, 3H), 1.29 (d, 3H, J=5.5 Hz), 1.16-1.95 (m, 18H), 2.04-2.14 (m, 1H), 3.17 (dd, 1H, J=9.5, 5.5 Hz), 3.33 (dd, 1H, J=12.0, 4.0 Hz), 3.70-3.76 (m, 1H), 3.82 (d, 1H, J=8.5 Hz), 3.96 (s, 1H), 4.78, 4.88 (ABX, 2H, J_{AB}=18.5 Hz, J_{AX}=J_{BX}⁼1.5 Hz), 4.99 (s, 1H), 5.71 (dt, 1H, J=10.5, 2.0 Hz), 5.86 (s, 1H), 5.90 (d, 1H, J=10.5 Hz); ¹³C NMR (100 MHz, CDCl₃) δ −4.4, −3.5, 9.8, 18.2(2C), 21.8, 23.8, 26.1, 26.67, 26.74, 27.3, 30.2, 30.7, 30.9, 32.6, 33.4, 35.2, 36.7, 41.6, 45.6, 56.8, 68.2, 70.0, 73.8, 73.9, 75.9, 86.2, 93.6, 117.8, 127.8, 133.1, 175.0, 175.2; mass spectrum (MALDI): m/e calcd for C₃₅H₅₆O₇SiNa⁺639.3688. found 639.3693.

Synthesis of D16:

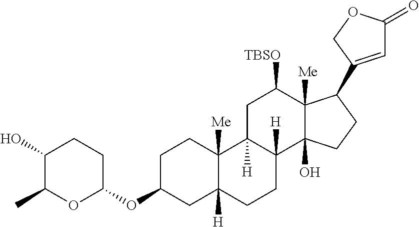

To a 0.5 ml NMM solution of 31 mg D15 was added 70 μl Et₃N and 109 mg NBSH at 0° C., the reaction was generally warmed up to room temperature and stirred for 24 hours. The reaction was quenched by saturated NaHCO₃ solution and extracted by EtOAc, dried over Na₂SO₄, and concentrated. The crude product was purified by flash chromatography with 50% EtOAc in hexanes to afford product D16 (25 mg, 81%). R_{f}=0.13 [40% EtOAc in hexanes]; mp 198-200° C.; [α]_{D}²⁰=−26.7 (c 1.8, CH₂Cl₂); IR (film) cm⁻¹ 3460 (m), 2930(m), 2858(m), 1739(s), 1621(w), 1448(m), 1380(m), 1254(m), 1109(m), 1049(s), 1028(s), 991(s), 881(m), 834(s), 773(m); ¹H NMR (400 MHz, CDCl₃) δ 0.05 (s, 3H), 0.06 (s, 3H), 0.79 (s, 3H), 0.90 (s, 9H), 0.92 (s, 3H), 1.22 (d, 3H, J=6.8 Hz), 1.16-1.96 (m, 22H), 2.03-2.11 (m, 1H), 3.17-3.19 (m, 1H), 3.23-3.27 (m, 1H), 3.33 (dd, 1H, J=11.2, 3.2 Hz), 3.59-3.66 (m, 1H), 3.91 (s, 1H), 4.78, 4.87 (AB, 2H, J_{AB}=17.6 Hz), 4.80 (s, 1H), 5.87 (s, 1H); ¹³C NMR (100 MHz, CDCl₃) δ −4.4, −3.6, 9.8, 18.1, 18.3, 21.8, 23.8, 26.1, 26.67, 26.72, 27.3, 27.9, 30.0, 30.3, 30.5, 30.8, 32.5, 33.4, 35.2, 36.6, 41.6, 45.6, 56.7, 69.9, 70.9, 72.6, 73.9, 76.0, 86.2, 94.3, 117.8, 175.1(2C); mass spectrum (MALDI): m/e calcd for C₃₅H₅₈O₇SiNa⁺641.3844. found 641.3880.

Synthesis of D17:

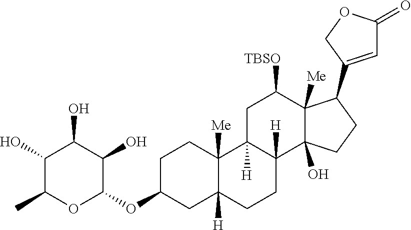

D15 (64 mg) was dissolved in t-BuOH/Acetone/CH₂Cl₂ (0.5 ml/0.5 ml/0.25 ml) and cooled to 0° C., NMO (50% w/v, 0.15 ml) was added, following by 0.5 mg OsO₄. The mixture was stirred at 0° C. for 18 hours. After the reaction was done, aqueous Na₂SO₃ solution was added, extracted by EtOAc, dried over Na₂SO₄, and concentrated. The crude product was purified by flash chromatography with 5% CH₃OH in CH₂Cl₂ to afford product D17 (62.6 mg, 96%). R_{f}=0.11 [5% CH₃OH in CHCl₃]; mp 286-288° C.; [α]_{D}²⁰=−7.8 (c 0.3, CH₃OH); IR (film) cm⁻¹ 3424 (s), 2929(m), 1738(s), 1623(w), 1448(w), 1381(w), 1254(m), 1047(s), 984(w), 881(m), 834(m); ¹H NMR (400 MHz, CD₃OD) δ 0.09 (s, 3H), 0.13 (s, 3H), 0.80 (s, 3H), 0.94 (s, 9H), 0.95 (s, 3H), 1.24 (d, 3H, J=6.0 Hz), 1.48-2.06 (m, 18H), 2.10-2.15 (m, 1H), 3.24-3.26 (m, 1H), 3.36 (dd, 1H, J=9.6, 9.6 Hz), 3.47-3.49 (m, 1H), 3.63-3.69 (m, 1H), 3.75 (s, 1H), 3.95 (s, 1H), 4.76 (s, 1H), 4.87, 4.98 (AB, 2H, J_{AB}=17.6 Hz), 5.81 (s, 1H); ¹³C NMR (100 MHz, CD₃OD) δ −5.7, −4.8, 9.1, 16.8, 17.7, 21.5, 23.0, 25.2, 26.1, 26.5, 26.9, 29.5, 30.3, 30.6, 32.1, 32.4, 35.0, 36.9, 40.9, 45.8, 56.8, 68.9, 71.3, 71.8, 72.3, 72.9, 74.3, 75.8, 85.6, 98.7, 116.5, 175.9, 177.4; mass spectrum (MALDI): m/e calcd for C₃₅H₅₈O₉SiNa⁺673.3742. found 673.3768.

### Example 3

### Synthesis of Hypoxia Sensitive Digoxin Prodrugs

The hypoxia sensitive Digoxin prodrug compounds were synthesized using the procedure depicted in Scheme 3:

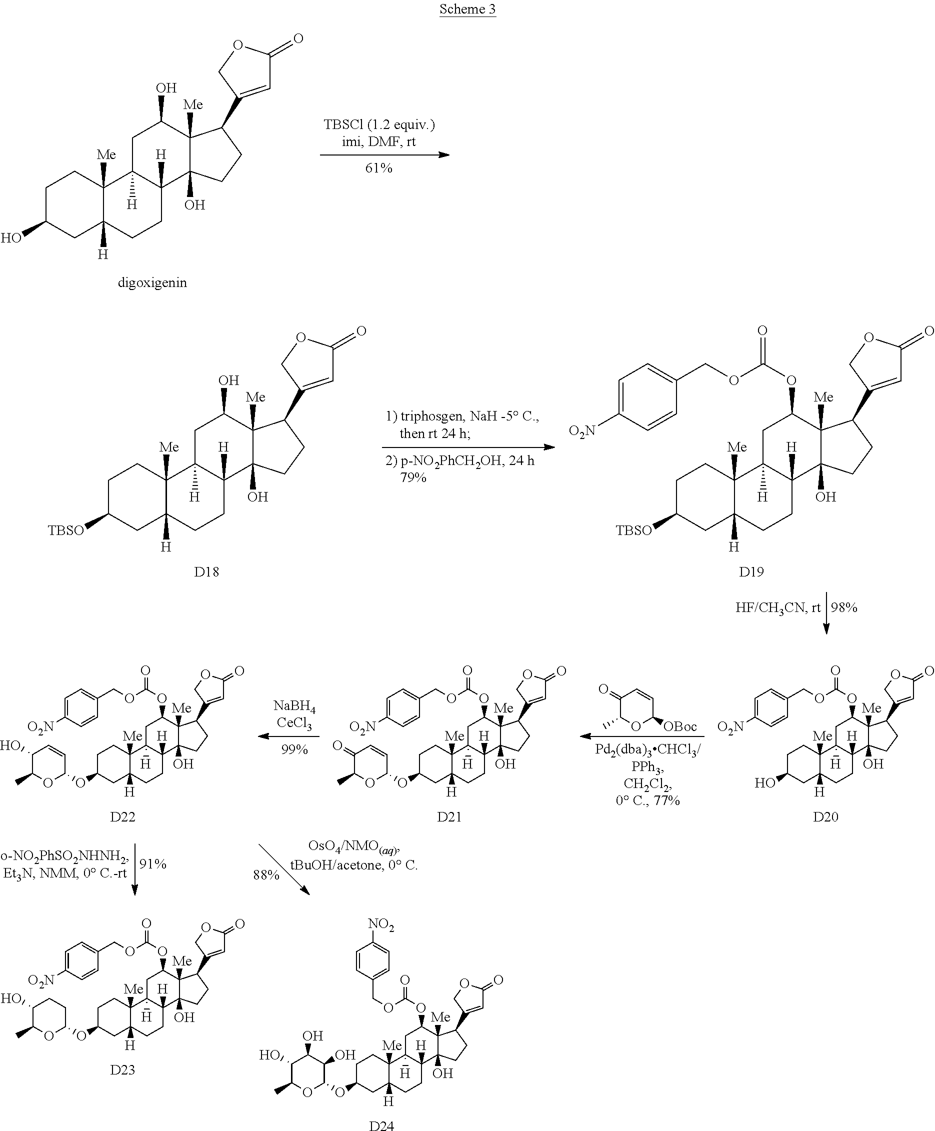

Synthesis of D18:

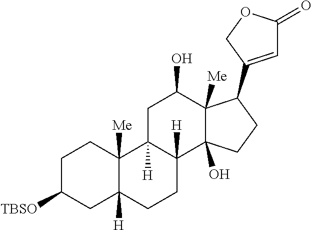

To a DMF (8 ml) solution of 390.5 mg digoxigenin and 272 mg imidazole at 0° C. was added 12.2 mg DMAP and 181 mg TBSCl. Then the reaction was stirred at 0° C. for 5 mins, then warmed up to room temperature and stirred for 24 hours. After the reaction was done, the mixture was quenched by H₂O and extracted by EtOAc threes times, then washed by H₂O five times, the organic phase was then dried over Na₂SO₄, and concentrated. The crude product was purified by flash chromatography with 40% EtOAc in hexanes to afford product D18 (310 mg, 61%, 67% bsrm), 31 mg digoxingenin was recovered. R_{f}=0.20 [40% EtOAc in hexanes]; mp 236-237° C.; [α]_{D}²⁰=+21.2 (c 0.9, CH₃OH); IR (film) cm⁻¹3483 (w), 2928(m), 2856(m), 1754(s), 1613(w), 1450(w), 1360(w), 1250(w), 1093(m), 1062(s), 1027(s); ¹H NMR (400 MHz, d₆-acetone) δ 0.05 (s, 6H), 0.83 (s, 3H), 0.90 (s, 9H), 0.94 (s, 3H), 1.19-2.13 (m, 19H), 3.32 (s, 1H), 3.38-3.46 (m, 2H), 3.82 (d, 1H, J=5.2 Hz), 4.12 (s, 1H), 4.82, 4.93 (ABX, 2H, J_{AB}=18.0 Hz, J_{AX}=J_{BX}=1.6 Hz), 5.82 (s, 1H); ¹³C NMR (100 MHz, d₆-acetone) δ −5.4, −5.3, 9.1, 18.0, 21.9, 23.7, 25.6, 27.0, 27.4, 28.6, 30.1, 30.2, 32.4, 33.0, 34.3, 35.3, 36.7, 41.4, 46.0, 56.1, 67.6, 73.4, 74.3, 85.3, 116.8, 173.9, 176.0; mass spectrum (ESI): m/e calcd for C₂₉H₄₉O₅Si⁺505.3349. found 505.3342.

Synthesis of D19:

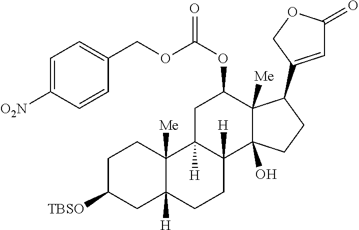

To a 1.5 ml THF solution of triphosgen (74 mg) was added dropwise of a 3.5 ml THF solution of D18 (252 mg) at −5° C., following with 60% NaH (70 mg). Then the mixture was warmed to room temperature and stirred at room temperature for 24 hours. Para-nitrobenzyl alcohol (153 mg) was added and stirred at room temperature for 24 hours. The reaction was quenched by saturated NH₄Cl solution and extracted by EtOAc, dried over Na₂SO₄, and concentrated. The crude product was purified by flash chromatography with 40% EtOAc in hexanes to afford product D19 (271 mg, 79%). R_{f}=0.24 [40% EtOAc in hexanes]; mp 188-190° C.; [α]_{D}²⁰=+42.9 (c 1.4, CH₂Cl₂); IR (film) cm⁻¹3479 (w), 2928(m), 2857(m), 1740(s), 1625(w), 1524(m), 1447(w), 1347(m), 1256(s), 1158(w), 1058(m), 960(m), 939(m), 837(m); ¹H NMR (400 MHz, CDCl₃) δ 0.01 (s, 6H), 0.87 (s, 9H), 0.89 (s, 3H), 0.93 (s, 3H), 1.25-1.96 (m, 18H), 2.15-2.17 (m, 1H), 2.96-2.97 (m, 1H), 4.04 (s, 1H), 4.44 (dd, 1H, J=12.0, 4.0 Hz), 4.77, 4.84 (AB, 2H, J_{AB}=17.6 Hz), 5.23, 5.28 (AB, 2H, J_{AB}=13.2 Hz), 5.85 (s, 1H), 7.55 (d, 2H, J=8.4 Hz), 8.26 (d, 2H, J=8.4 Hz); ¹³C NMR (100 MHz, CDCl₃) δ −4.6, 10.5, 18.3, 21.9, 23.9, 26.0, 26.6, 26.7, 27.4, 28.8, 29.9, 32.5, 33.3, 34.4, 35.5, 36.2, 41.7, 46.2, 54.4, 67.1, 68.3, 73.5, 82.9, 86.1, 118.4, 124.2, 128.7, 142.4, 148.2, 155.1, 173.1, 174.4; mass spectrum (MALDI): m/e calcd for C₃₂H₅₃O₉NSiNa⁺706.3382. found 706.3415.

Synthesis of D20:

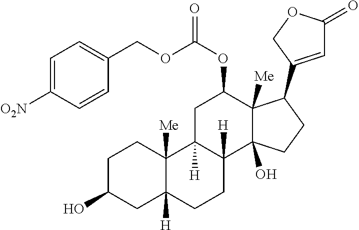

To a 0.1 ml CH₃CN solution of TBS ether D19 (10.4 mg) was added 5% HF in CH₃CN (750 at room temperature, after stirring for overnight, another 75 μl of 5% HF in CH₃CN was added and the mixture was stirred at room temperature for 24 hours. After the reaction was done, saturated NaHCO₃ solution was added and extracted with EtOAc, the organic phase was combined and dried over Na₂SO₄. The crude product was purified by flash chromatography with 60% EtOAc in hexanes to afford product D20 (8.5 mg, 98%). R_{f}=0.12 [60% EtOAc in hexanes]; mp 225-228° C.; [α]_{D}²⁰=+43.8 (c 0.9, CH₂Cl₂); IR (film) cm⁻¹ 3459 (w), 2929(m), 1737(s), 1625(w), 1523(m), 1449(w), 1383(w), 1347(m), 1260(s), 1177(w), 1109(w), 1028(m), 966(m), 854(w), 737(m); ¹H NMR (400 MHz, CDCl₃) δ 0.91 (s, 3H), 0.97 (s, 3H), 1.24-1.96 (m, 18H), 2.15-2.22 (m, 1H), 2.98 (m, 1H), 4.15 (s, 1H), 4.45 (dd, 1H, J=11.6, 4.4 Hz), 4.78, 4.85 (AB, 2H, J_{AB}=17.6 Hz), 5.24, 5.29 (AB, 2H, J_{AB}=13.2 Hz), 5.87 (s, 1H), 7.56 (d, 2H, J=8.0 Hz), 8.27 (d, 2H, J=8.8 Hz); ¹³C NMR (100 MHz, CDCl₃) δ 10.5, 21.8, 23.7, 26.5, 26.6, 27.4, 28.1, 29.8, 32.3, 33.3, 33.5, 35.6, 36.1, 41.6, 46.2, 54.4, 66.8, 68.3, 73.5, 82.8, 86.0, 118.5, 124.2, 128.7, 142.3, 148.3, 155.1, 173.1, 174.4; mass spectrum (MALDI): m/e calcd for C₃₁H₃₉O₉NNa⁺592.2517. found 592.2515.

Synthesis of D21:

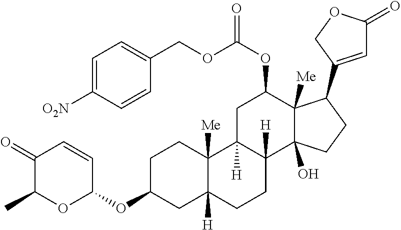

31 mg D20 and 50 mg tert-butyl ((2R,6R)-6-methyl-5-oxo-5,6-dihydro-2H-pyran-2-yl) carbonate was dissolved in CH₂Cl₂ (0.5 ml), and cooled to 0° C. 2.8 mg Pd₂(DBA)₃.CHCl₃ and 2.9 mg Ph₃P was dissolved in 0.4 ml CH₂Cl₂ and was added to the above mixture at 0° C. The reaction mixture was stirred at 0° C. for 30 hours, and then was quenched by saturated NaHCO₃ solution and extracted by EtOAc, dried over Na₂SO₄, and concentrated. The crude product was purified by flash chromatography with 50% EtOAc in hexanes to afford product D21 (28.4 mg, 77%). R_{f}=0.29 [60% EtOAc in hexanes]; mp 102-106° C.; [α]_{D}²⁰=+61.3 (c 2.1, CH₂Cl₂); IR (film) cm⁻¹3481 (w), 2931(m), 1740(s), 1698 (m), 1627(w), 1523(m), 1448(w), 1382(w), 1347(m), 1260(s), 1030(s), 964(m), 943(m); ¹H NMR (400 MHz, CDCl₃) δ 0.89 (s, 3H), 0.94 (s, 3H), 1.23-1.95 (m, 18H), 1.35 (d, 3H, J=6.8 Hz), 2.15-2.21 (m, 1H), 2.95-2.98 (m, 1H), 4.07 (s, 1H), 4.44 (dd, 1H, J=12.0, 4.0 Hz), 4.54 (q, 1H, J=6.8 Hz), 4.77, 4.85 (AB, 2H, J_{AB}=18.4 Hz), 5.23, 5.27 (AB, 2H, J_{AB}=13.2 Hz), 5.25 (s, 1H), 5.86 (s, 1H), 6.06 (d, 1H, J=10.0 Hz), 6.81 (dd, 1H, J=10.0, 3.2 Hz), 7.55 (d, 2H, J=8.0 Hz), 8.25 (d, 2H, J=8.0 Hz); ¹³C NMR (100 MHz, CDCl₃) δ 10.5, 15.5, 21.7, 23.8, 26.6, 26.7, 27.4, 29.9, 30.5, 30.7, 32.5, 33.3, 35.4, 36.6, 41.5, 46.1, 54.4, 68.3, 70.7, 73.6, 74.2, 82.7, 85.9, 92.2, 118.4, 124.2, 127.3, 128.7, 142.3, 144.5, 148.2, 155.1, 173.3, 174.5, 197.5; mass spectrum (MALDI): m/e calcd for C₃₇H₄₅O₁₁NNa⁺702.2885. found 702.2883.

Synthesis of D22:

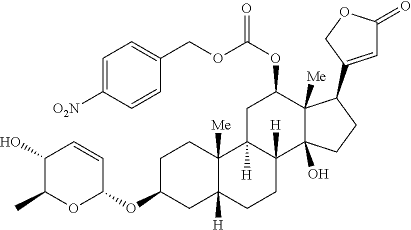

To a 0.1 ml CH₂Cl₂ solution of 6.8 mg D21 was added a solution of CeCl₃ in CH₃OH (0.4M, 0.1 ml), the reaction was then cooled to −78° C., and 4 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 60% EtOAc in hexanes to afford product D22 (6.8 mg, 99%). R_{f}=0.20 [50% EtOAc in hexanes]; mp 116-119° C.; [α]_{D}²⁰=+28.9 (c 1.4, CH₂Cl₂); IR (film) cm⁻¹ 3436 (m), 2933(m), 1734(s), 1624(w), 1523(m), 1448(w), 1382(w), 1347(m), 1258(s), 1029(s), 1000(s), 962(m), 941(m), 732(s), 699(m); ¹H NMR (400 MHz, CDCl₃) δ 0.89 (s, 3H), 0.93 (s, 3H), 1.24-1.95 (m, 18H), 1.28 (d, 3H, J=6.0 Hz), 2.14-2.21 (m, 1H), 2.97 (m, 1H), 3.70-3.83 (m, 1H), 3.83 (m, 1H), 3.98 (s, 1H), 4.44 (dd, 1H, J=12.0, 4.0 Hz), 4.78, 4.84 (AB, 2H, J_{AB}=18.4 Hz), 5.00 (s, 1H), 5.23, 5.28 (AB, 2H, J_{AB}=13.2 Hz), 5.72 (d, 1H, J=10.4 Hz), 5.86 (s, 1H), 5.91 (d, 1H, J=10.4 Hz), 7.55 (d, 2H, J=8.4 Hz), 8.26 (d, 2H, J=8.0 Hz); ¹³C NMR (100 MHz, CDCl₃) δ 10.5, 18.2, 21.3, 23.8, 26.58, 26.63, 26.9, 27.4, 30.5, 30.9, 32.5, 33.3, 35.4, 36.6, 41.5, 46.1, 54.4, 68.2, 68.3, 69.9, 73.6, 73.7, 82.8, 86.0, 93.5, 118.4, 124.2, 127.7, 128.7, 133.2, 142.3, 148.2, 155.1, 173.4, 174.6 mass spectrum (MALDI): m/e calcd for C₃₂H₄₂O₁₁NNa⁺704.3041. found 704.3051.

Synthesis of D23:

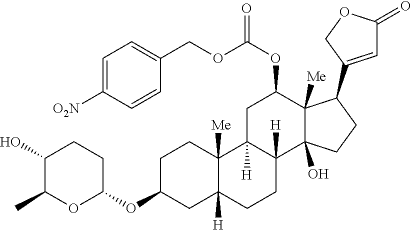

To a 0.2 ml NMM solution of 7 mg D22 was added 15 μl Et₃N and 22 mg NBSH at 0° C., the reaction was generally warmed up to room temperature and stirred for 24 hours. The reaction was quenched by saturated NaHCO₃ solution and extracted by EtOAc, dried over Na₂SO₄, and concentrated. The crude product was purified by flash chromatography with 60% EtOAc in hexanes to afford product D23 (6.4 mg, 91%). R_{f}=0.24 [60% EtOAc in hexanes]; mp 92-96° C.; [α]_{D}²⁰=+3.1 (c 0.5, CH₂Cl₂); IR (film) cm⁻¹ 3450 (m), 2927(m), 1738(s), 1624(w), 1524(m), 1449(w), 1382(w), 1347(m), 1260(s), 1029(m), 991(m); ¹H NMR (400 MHz, CDCl₃) δ 0.89 (s, 3H), 0.94 (s, 3H), 1.21-1.95 (m, 20H), 1.22 (d, 3H, J=6.8 Hz), 2.14-2.21 (m, 1H), 2.97 (m, 1H), 3.26 (m, 1H), 3.58-3.65 (m, 1H), 3.91 (s, 1H), 4.43-4.46 (m, 1H), 4.77, 4.84 (AB, 2H, J_{AB}=18.0 Hz), 4.80 (s, 1H), 5.23, 5.28 (AB, 2H, J_{AB}=13.2 Hz), 5.86 (s, 1H), 7.55 (d, 2H, J=8.0 Hz), 8.26 (d, 2H, J=8.0 Hz); ¹³C NMR (100 MHz, CDCl₃) δ 10.5, 18.1, 21.8, 23.8, 26.6, 26.7, 26.8, 27.4, 28.0, 30.1, 30.4, 30.7, 32.5, 33.4, 35.4, 36.6, 41.6, 46.1, 54.4, 68.3, 69.8, 70.9, 72.5, 73.5, 82.8, 86.0, 94.4, 118.4, 124.2, 128.7, 142.3, 148.2, 155.1, 173.2, 174.5; mass spectrum (MALDI): m/e calcd for C₃₂H₄₉O₁₁NNa⁺706.3198. found 706.3187.

### Example 4

### Alternate Synthesis of Hypoxia Sensitive Digoxin Prodrugs

Some hypoxia sensitive Digoxin prodrug compounds were synthesized using the alternate procedure depicted in Scheme 4:

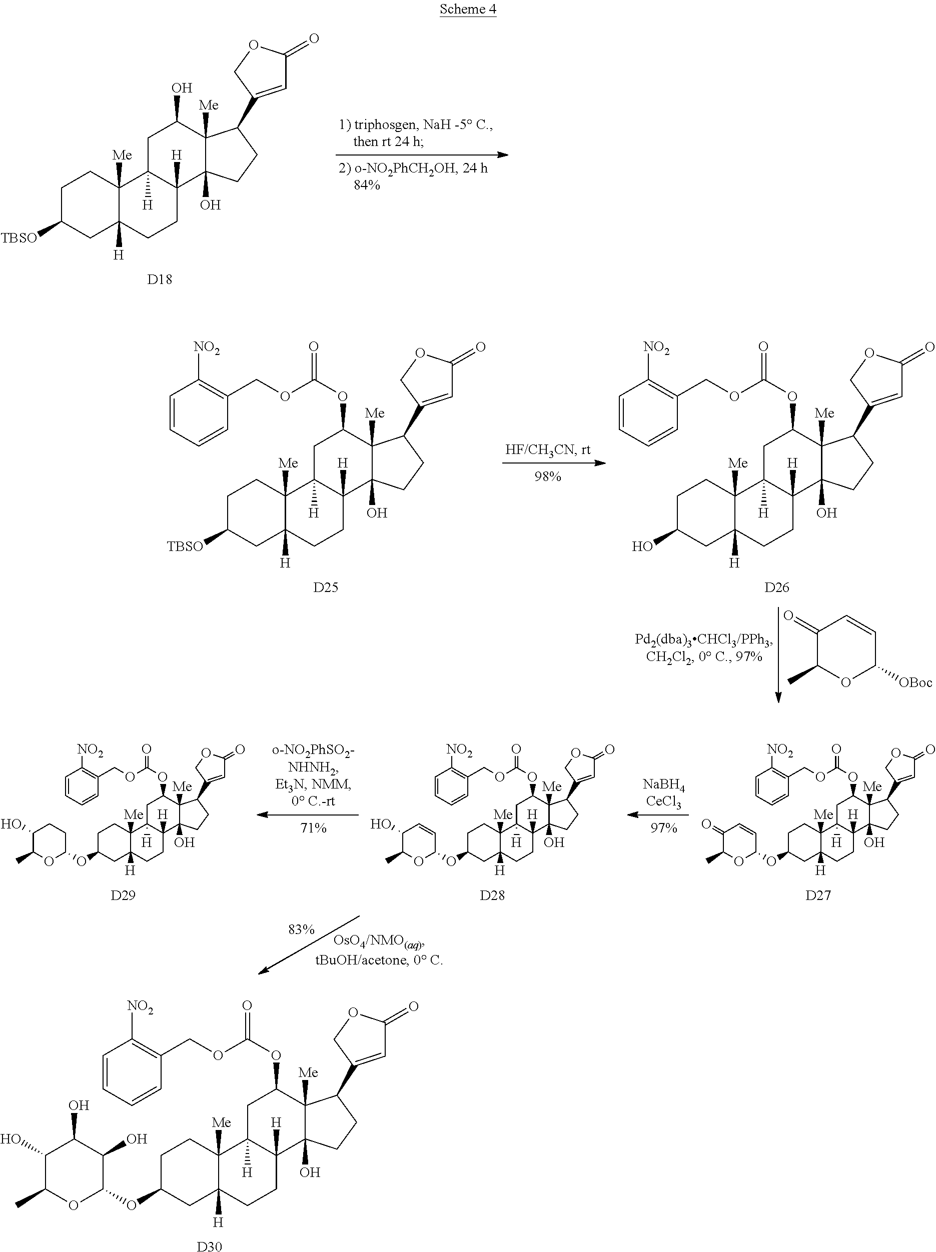

Synthesis of D24:

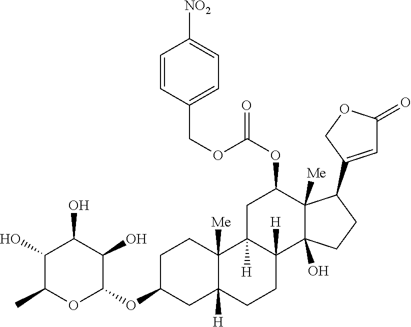

D22 (7 mg) was dissolved in t-BuOH/Acetone (0.1 ml/0.1 ml) and cooled to 0° C., NMO (50% w/v, 25 μl) was added, following by 0.2 mg OsO₄. The mixture was stirred at 0° C. for 18 hours. After the reaction was done, aqueous Na₂SO₃ solution was added, extracted by EtOAc, dried over Na₂SO₄, and concentrated. The crude product was purified by flash chromatography with 5% CH₃OH in CH₂Cl₂ to afford product D24 (6.5 mg, 88%). R_{f}=0.10 [5% MeOH in CHCl₃]; mp 90-95° C.; [α]_{D}²⁰=+6.2 (c 0.5, CH₂Cl₂/MeOH: 4/1); IR (film) cm⁻¹ 3405 (m), 2928(m), 1738(s), 1625(w), 1523(m), 1449(w), 1383(w), 1347(m), 1261(s), 1048(m); ¹H NMR (400 MHz, CD₃OD) δ 0.88 (s, 3H), 0.96 (s, 3H), 1.24-2.04 (m, 18H), 1.23 (d, 3H, J=6.8 Hz), 2.15-2.20 (m, 1H), 3.06-3.08 (m, 1H), 3.36 (dd, 1H, J=9.6, 9.6 Hz), 3.62-3.69 (m, 2H), 3.75 (s, 1H), 3.95 (s, 1H), 4.50-4.53 (m, 1H), 4.76 (s, 1H), 4.84-4.96 (m, 2H), 5.31 (s, 2H), 5.81 (s, 1H), 7.65 (d, 2H, J=8.8 Hz), 8.27 (d, 2H, J=8.8 Hz); ¹³C NMR (100 MHz, CD₃OD) δ 9.7, 16.8, 21.5, 23.0, 26.2, 26.3, 26.5, 27.2, 29.5, 30.4, 32.1, 32.2, 35.2, 36.9, 41.0, 46.0, 54.5, 68.0, 68.9, 71.3, 71.7, 72.2, 72.9, 74.0, 82.5, 85.4, 98.7, 117.0, 123.6, 128.6, 143.3, 148.2, 155.1, 175.8(2C); mass spectrum (MALDI): m/e calcd for C₃₂H₄₉O₁₃NNa⁺738.3096. found 738.3097.

Synthesis of D25:

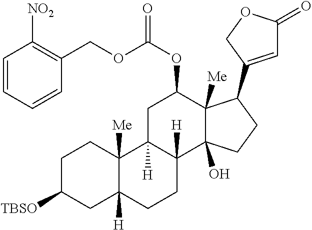

To a 1.5 ml THF solution of triphosgen (78.5 mg) was added dropwise of a 3.5 ml THF solution of D18 (267 mg) at −5° C., following with 60% NaH (74 mg). Then the mixture was warmed to room temperature and stirred at room temperature for 24 hours. 2-nitrobenzyl alcohol (162 mg) was added and stirred at room temperature for 24 hours. The reaction was quenched by saturated NH₄Cl solution and extracted by EtOAc, dried over Na₂SO₄, and concentrated. The crude product was purified by flash chromatography with 25% EtOAc in hexanes to afford product D25 (303 mg, 84%). R_{f}=0.29 [40% EtOAc in hexanes]; mp 105-107° C.; [α]_{D}²⁰+50.9 (c 2.9, CH₂Cl₂); IR (film) cm⁻¹3455 (w), 2929 (m), 2857(w), 1738(s), 1528(m), 1446(w), 1382(w), 1343(m), 1251(s), 1158(w), 1056(m), 960(m), 939(m); ¹H NMR (400 MHz, CDCl₃) δ 0.00 (s, 6H), 0.86 (s, 9H), 0.89 (s, 3H), 0.92 (s, 3H), 1.20-1.95 (m, 18H), 2.15-2.21 (m, 1H), 2.98-2.99 (m, 1H), 4.03 (s, 1H), 4.47 (dd, 1H, J=12.0, 3.6 Hz), 4.78, 4.88 (AB, 2H, J_{AB}=18.0 Hz), 5.49, 5.65 (AB, 2H, J_{AB}=14.8 Hz), 5.89 (s, 1H), 7.54 (dd, 1H, J=8.0, 7.6 Hz), 7.62 (d, 1H, J=8.0 Hz), 7.70 (dd, 1H, J=7.6, 7.2 Hz), 8.14 (d, 2H, J=8.0 Hz); ¹³C NMR (100 MHz, CDCl₃) δ −4.6, 10.5, 18.3, 21.9, 23.9, 26.0, 26.6, 26.8, 27.6, 28.8, 29.9, 32.4, 33.3, 34.4, 35.5, 36.2, 41.6, 46.1, 54.4, 66.6, 67.1, 73.7, 82.7, 86.1, 118.3, 125.5, 129.1, 129.5, 131.6, 134.2, 147.6, 154.9, 173.7, 174.7; mass spectrum (MALDI): m/e calcd for C₃₇H₅₃O₉NSiNa⁺706.3382. found 706.3351.

Synthesis of D26:

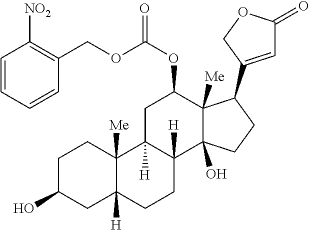

To a 1 ml CH₃CN solution of TBS ether D25 (303 mg) was added 5% HF in CH₃CN (4.5 ml) at room temperature, and the mixture was stirred at room temperature for 24 hours. After the reaction was done, saturated NaHCO₃ solution was added and extracted with EtOAc, the organic phase was combined and dried over Na₂SO₄. The crude product was purified by flash chromatography with 60% EtOAc in hexanes to afford product D26 (246.5 mg, 98%). R_{f}=0.17 [60% EtOAc in hexanes]; mp 129-132° C.; [α]_{D}²⁰=+57.0 (c 1.9, CH₂Cl₂); IR (film) cm⁻¹ 3459 (w), 2930(m), 1738(s), 1528(m), 1447(w), 1382(w), 1343(m), 1259(s), 1028(m), 965(m), 941(m); ¹H NMR (400 MHz, CDCl₃) δ 0.92 (s, 3H), 0.97 (s, 3H), 1.26-1.98 (m, 18H), 2.17-2.22 (m, 1H), 3.00 (m, 1H), 4.15 (s, 1H), 4.49 (dd, 1H, J=12.4, 4.4 Hz), 4.79, 4.88 (AB, 2H, J_{AB}=18.0 Hz), 5.51, 5.67 (AB, 2H, J_{AB}=14.4 Hz), 5.91 (s, 1H), 7.56 (dd, 1H, J=8.0, 7.2 Hz), 7.64 (d, 1H, J=8.0 Hz), 7.72 (dd, 1H, J=7.6, 7.2 Hz), 8.15 (d, 1H, J=8.4 Hz); ¹³C NMR (100 MHz, CDCl₃) δ 10.5, 21.8, 23.8, 26.5, 26.6, 27.6, 28.1, 29.8, 32.3, 33.36, 33.42, 35.6, 36.1, 41.6, 46.1, 54.4, 66.6, 66.8, 73.7, 82.6, 86.1, 118.4, 125.5, 129.2, 129.6, 131.5, 134.2, 147.7, 154.9, 173.3, 174.6; mass spectrum (MALDI): m/e calcd for C₃₁H₃₉O₉NNa⁺592.2517. found 592.2538.

Synthesis of D27:

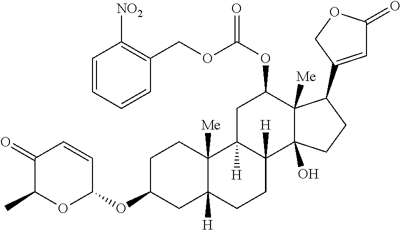

114 mg D26 and 183 mg tert-butyl ((2S,6S)-6-methyl-5-oxo-5,6-dihydro-2H-pyran-2-yl) carbonate was dissolved in CH₂Cl₂ (2 ml), and cooled to 0° C. 10.4 mg Pd₂(DBA)₃.CHCl₃ and 10.5 mg Ph₃P was dissolved in 0.5 ml CH₂Cl₂ and was added to the above mixture at 0° C. The reaction mixture was stirred at 0° C. for 8 hours, and then was quenched by saturated NaHCO₃ solution and extracted by EtOAc, dried over Na₂SO₄, and concentrated. The crude product was purified by flash chromatography with 50% EtOAc in hexanes to afford product D27 (131.6 mg, 97%). R_{f}=0.35 [60% EtOAc in hexanes]; mp 110-114° C.; [α]_{D}²⁰=+60.8 (c 1.8, CH₂Cl₂); IR (film) cm⁻¹ 3483 (w), 2938(m), 1743(s), 1700(m), 1629(w), 1528(s), 1448(m), 1382(m), 1344(m), 1261(s), 1031(m); ¹H NMR (400 MHz, CDCl₃) δ 0.88 (s, 3H), 0.92 (s, 3H), 1.20-1.95 (m, 18H), 1.33 (d, 3H, J=6.4 Hz), 2.15-2.20 (m, 1H), 2.96-2.98 (m, 1H), 4.05 (s, 1H), 4.46 (dd, 1H, J=12.0, 3.6 Hz), 4.52 (q, 1H, J=6.4 Hz), 4.77, 4.87 (AB, 2H, J_{AB}=18.4 Hz), 5.23 (s, 1H), 5.47, 5.63 (AB, 2H, J_{AB}=14.4 Hz), 5.88 (s, 1H), 6.03 (d, 1H, J=10.0 Hz), 6.79 (dd, 1H, J=10.0, 2.4 Hz), 7.52 (dd, 1H, J=8.0, 7.2 Hz), 7.61 (d, 1H, J=7.2 Hz), 7.69 (dd, 1H, J=8.0, 7.2 Hz), 8.11 (d, 1H, J=8.0 Hz); ¹³C NMR (100 MHz, CDCl₃) δ 10.5, 15.4, 21.7, 23.8, 26.58, 26.63, 26.7, 27.6, 30.5, 30.7, 32.5, 33.2, 35.4, 36.7, 41.4, 46.0, 54.5, 66.6, 70.6, 73.8, 74.3, 82.5, 85.9, 92.2, 118.2, 125.5, 127.2, 129.2, 129.6, 131.5, 134.2, 144.6, 147.6, 154.9, 173.9, 174.9, 197.6; mass spectrum (MALDI): m/e calcd for C₃₇H₄₅O₁₁NNa⁺702.2885. found 702.2897.

Synthesis of D28:

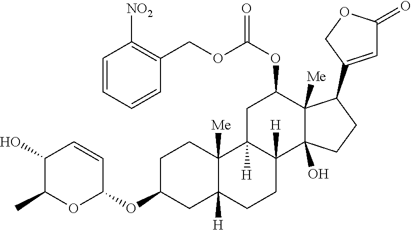

To a 0.3 ml CH₂Cl₂ solution of 31.6 mg D27 was added a solution of CeCl₃ in CH₃OH (0.4M, 0.1 ml), the reaction was then cooled to −78° C., and 9 mg NaBH₄ was added. The reaction was stirred at −78° C. for 1 hour. After the reaction was done, the mixture was quenched by saturated NaHCO₃ solution and extracted by EtOAc, dried over Na₂SO₄, and concentrated. The crude product was purified by flash chromatography with 60% EtOAc in hexanes to afford product D28 (30.6 mg, 97%). R_{f}=0.10 [50% EtOAc in hexanes]; mp 211-213° C.; [α]_{D}²⁰=+32.5 (c 3.0, CH₂Cl₂); IR (film) cm⁻¹ 3436 (m), 2938(m), 1744(s), 1529(s), 1448(m), 1382(m), 1344(m), 1261(s), 1031(s); ¹H NMR (400 MHz, CDCl₃) δ 0.89 (s, 3H), 0.93 (s, 3H), 1.21-1.98 (m, 18H), 1.28 (d, 3H, J=6.0 Hz), 2.15-2.21 (m, 1H), 2.97-3.00 (m, 1H), 3.70-3.74 (m, 1H), 3.81-3.84 (m, 1H), 3.97 (s, 1H), 4.47 (dd, 1H, J=11.6, 2.8 Hz), 4.78, 4.87 (AB, 2H, J_{AB}=17.6 Hz), 4.99 (s, 1H), 5.49, 5.65 (AB, 2H, J_{AB}=14.8 Hz), 5.71 (d, 1H, J=10.4 Hz), 5.89 (s, 2H), 7.54 (dd, 1H, J=8.0, 7.2 Hz), 7.62 (d, 1H, J=7.2 Hz), 7.70 (dd, 1H, J=8.0, 7.2 Hz), 8.14 (d, 1H, J=8.0 Hz); ¹³C NMR (100 MHz, CDCl₃) δ 10.5, 18.2, 21.8, 23.8, 26.6, 26.7, 26.9, 27.6, 30.5, 30.9, 32.5, 33.3, 35.4, 36.7, 41.6, 46.1, 54.4, 66.6, 68.2, 70.0, 73.69, 73.74, 82.6, 86.0, 93.5, 118.3, 125.5, 127.7, 129.1, 129.5, 131.5, 133.2, 134.2, 147.6, 154.9, 173.6, 174.8; mass spectrum (MALDI): m/e calcd for C₃₇H₄₇O₁₁NNa⁺704.3041. found 704.3049.

Synthesis of D29:

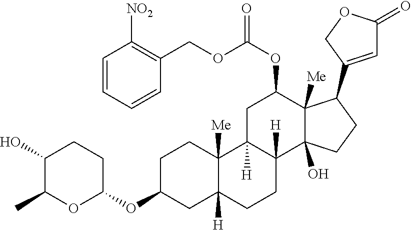

To a 0.6 ml NMM solution of 40 mg D28 was added 0.1 ml Et₃N and 126 mg NBSH at 0° C., the reaction was generally warmed up to room temperature and stirred for 24 hours. The reaction was quenched by saturated NaHCO₃ solution and extracted by EtOAc, dried over Na₂SO₄, and concentrated. The crude product was purified by flash chromatography with 60% EtOAc in hexanes to afford product D29 (28.3 mg, 71%). R_{f}=0.21 [60% EtOAc in hexanes]; mp 210-212° C.; [α]_{D}²⁰=+7.4 (c 2.8, CH₂Cl₂); IR (film) cm⁻¹ 3446 (m), 2935(m), 1742(s), 1529(m), 1344(m), 1261(s), 1031(m); ¹H NMR (400 MHz, CDCl₃) δ 0.89 (s, 3H), 0.93 (s, 3H), 1.21-1.95 (m, 20H), 1.21 (d, 3H, J=6.0 Hz), 2.15-2.21 (m, 1H), 2.98 (m, 1H), 3.22-3.27 (m, 1H), 3.59-3.63 (m, 1H), 3.90 (s, 1H), 4.47 (d, 1H, J=11.2 Hz), 4.78, 4.87 (AB, 2H, J_{AB}=18.4 Hz), 4.80 (s, 1H), 5.49, 5.65 (AB, 2H, J_{AB}=14.8 Hz), 5.89 (s, 1H), 7.54 (dd, 1H, J=8.0, 7.2 Hz), 7.62 (d, 1H, J=7.2 Hz), 7.70 (dd, 1H, J=7.2, 7.2 Hz), 8.13 (d, 1H, J=8.0 Hz); ¹³C NMR (100 MHz, CDCl₃) δ 10.5, 18.1, 21.8, 23.8, 26.6, 26.7, 26.8, 27.6, 27.9, 30.1, 30.5, 30.7, 32.5, 33.3, 35.4, 36.6, 41.6, 46.0, 54.4, 66.6, 69.8, 71.0, 72.5, 73.7, 82.6, 86.0, 94.4, 118.3, 125.5, 129.1, 129.5, 131.5, 134.2, 147.6, 154.9, 173.6, 174.7; mass spectrum (MALDI): m/e calcd for C₃₇H₄₉O₁₁NNa⁺706.3198. found 706.3216.

Synthesis of D30:

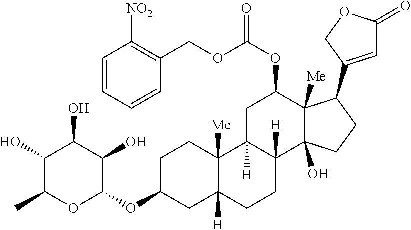

D28 (39.3 mg) was dissolved in t-BuOH/Acetone/CH₂Cl₂ (0.5 ml/0.5 ml/0.2 ml) and cooled to 0° C., NMO (50% w/v, 0.12 ml) was added, following by 0.8 mg OsO₄. The mixture was stirred at 0° C. for 8 hours. After the reaction was done, aqueous Na₂SO₃ solution was added, extracted by EtOAc, dried over Na₂SO₄, and concentrated. The crude product was purified by flash chromatography with 5% CH₃OH in CH₂Cl₂ to afford product D30 (34.2 mg, 83%). R_{f}=0.09 [5% MeOH in CHCl₃]; mp 218-221° C.; [α]_{D}²⁰=+15.1 (c 2.7, CH₂Cl₂/MeOH: 4/1); IR (film) cm⁻¹ 3436 (s), 2933(m), 1739(s), 1529(s), 1448(m), 1383(m), 1344(m), 1260(s), 1047(s); ¹H NMR (400 MHz, CD₃OD) δ 0.88 (s, 3H), 0.96 (s, 3H), 1.23-2.04 (m, 18H), 1.23 (d, 3H, J=6.0 Hz), 2.15-2.20 (m, 1H), 3.05-3.08 (m, 1H), 3.36 (dd, 1H, J=9.6, 9.6 Hz), 3.62-3.69 (m, 2H), 3.75 (s, 1H), 3.94 (s, 1H), 4.52 (d, 1H, J=9.6 Hz), 4.76 (s, 1H), 4.87, 4.96 (AB, 2H, J_{AB}=18.4 Hz), 5.46, 5.62 (AB, 2H, J_{AB}=14.0 Hz), 5.89 (s, 1H), 7.60 (dd, 1H, J=8.0, 7.2 Hz), 7.68 (d, 1H, J=7.2 Hz), 7.75 (dd, 1H, J=8.0, 7.2 Hz), 8.11 (d, 1H, J=8.0 Hz); ¹³C NMR (100 MHz, CD₃OD) δ 9.7, 16.8, 21.5, 23.0, 26.2, 26.3, 26.5, 27.2, 29.5, 30.4, 32.1, 32.3, 35.2, 36.9, 40.9, 46.0, 54.6, 66.3, 68.9, 71.3, 71.7, 72.2, 72.9, 74.1, 82.4, 85.4, 98.6, 117.1, 124.9, 129.5, 129.6, 131.2, 133.8, 148.1, 155.0, 175.81, 175.85; mass spectrum (MALDI): m/e calcd for C₃₇H₄₉O₁₃NNa⁺738.3096. found 738.3094.

### Example 5

### Synthesis of Oligosaccharide Compounds

The oligosaccharide compounds were synthesized using the procedure depicted in Scheme 5:

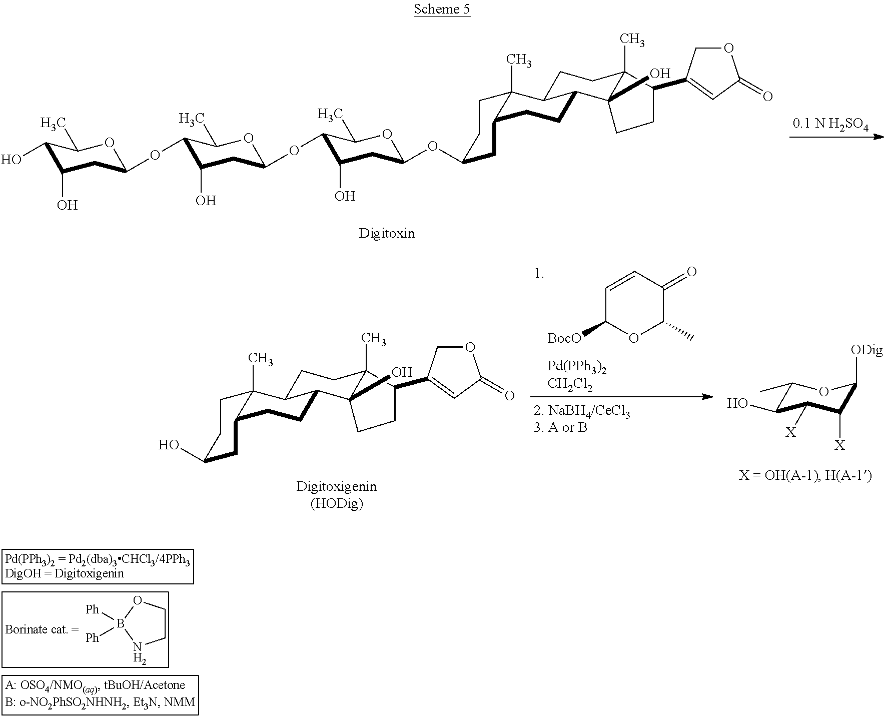

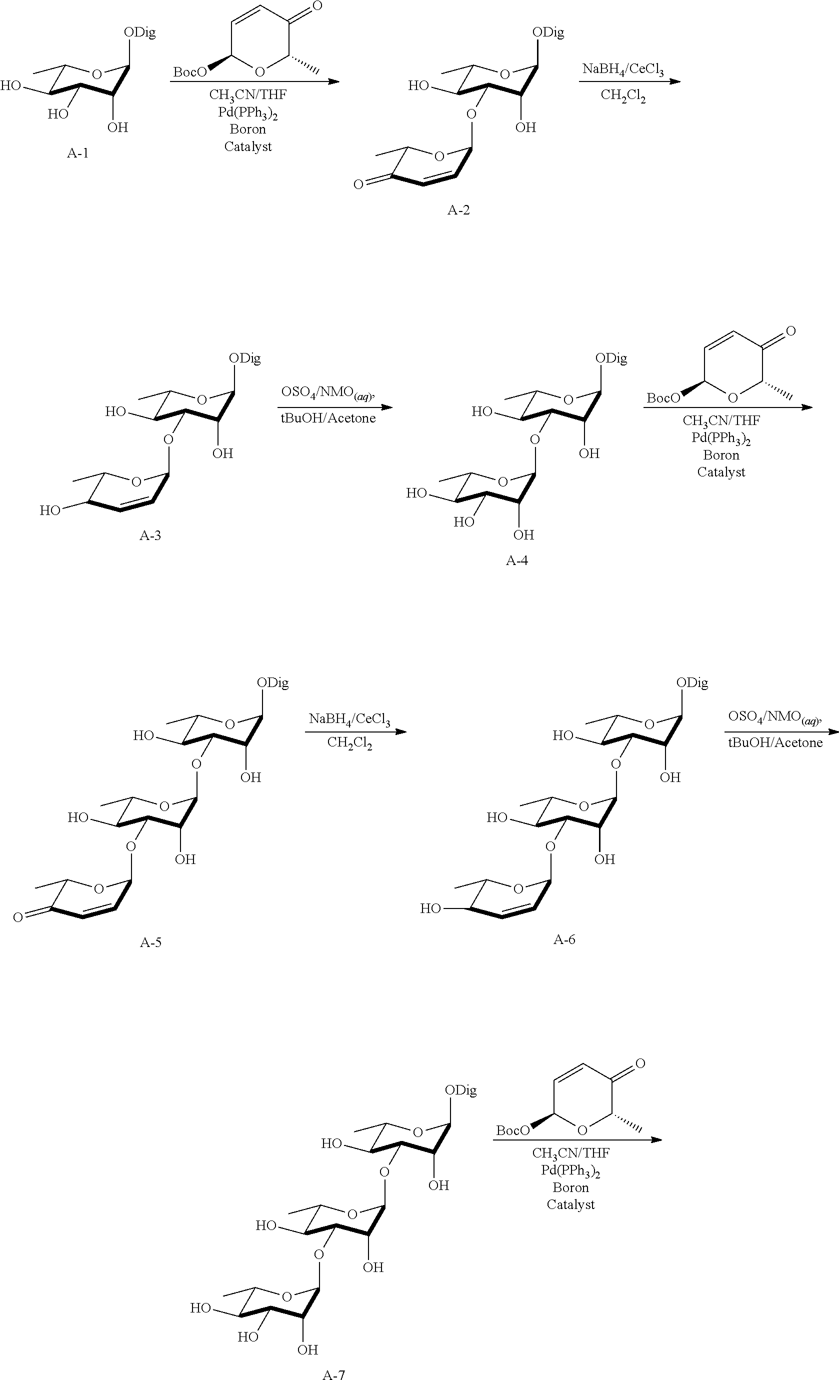

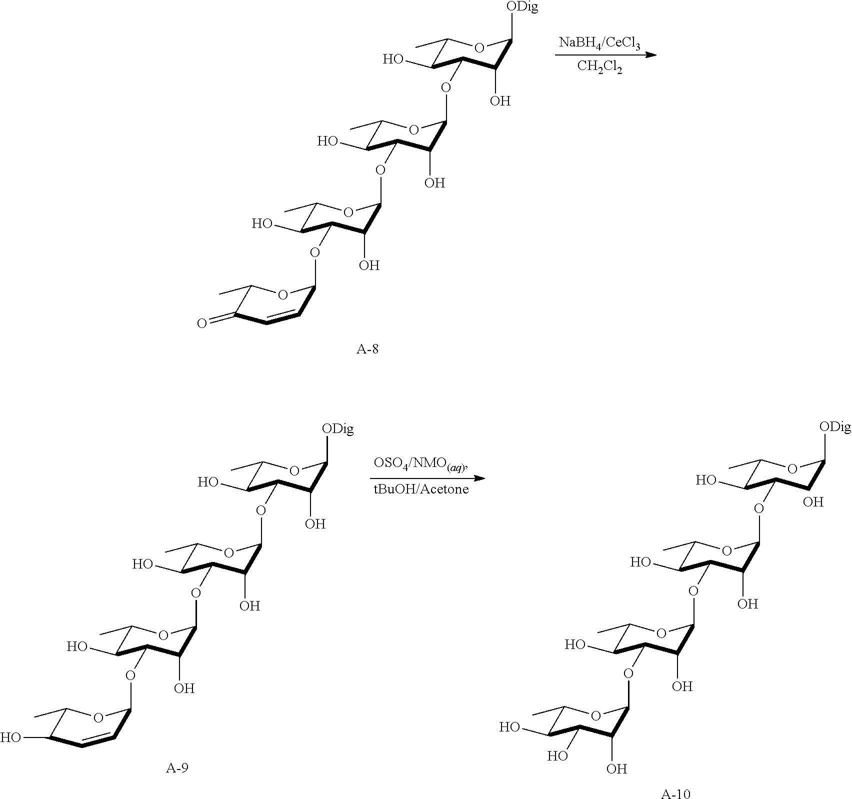

### Synthesis of [2,3-didehydro-4-oxo-α-L-Rhap-(1→3)-2,4-dihydroxy-α-L-Rhap]-Dig-2)

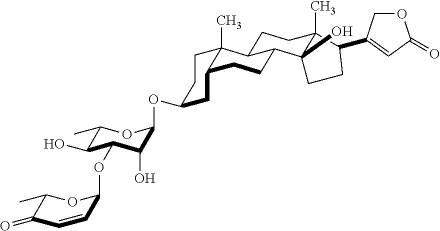

To rhamnose A-1 (60 mg, 0.115 mmol) dissolved in CH₃CN/THF (1:0.2) (2.2 mL, 0.05M) was added boron catalyst (3.9 mg, 0.017 mmol) and stirred at 0° C. for 20 min then added Boc-pyranone (35 mg, 0.15 mmol) followed by the addition of Pd₂(dba)₂.CHCl₃ (6 mg, 5 mol %) and PPh₃ (6.1 mg, 20 mol %) solution in CH₃CN/THF at 0 under argon atmosphere. Reaction was continued to stir for 5 h. The reaction mixture was diluted with EtOAc (5 mL) and quenched with 5 mL saturated NaHCO₃ solution, extracted with EtOAc (3×5 mL), dried with Na₂SO₄, and concentrated under reduced pressure. The crude product was purified using silica gel flash chromatography, eluting with 65-70% EtOAc/hexanes to give disaccharide enone A-2 (49 mg, 0.078 mmol, 67%) as a white sticky solid; R; (100% EtOAc)=0.5; [α]²⁵_{D}=−23 (C=0.67, CH₂Cl₂); IR (thin film, cm⁻¹) 3350, 2986, 2833, 1733, 1373, 1320, 1240, 1182, 1122, 1045, 1024, 733, 703; ¹H NMR (400 MHz, CDCl₃) δ 6.91 (dd, J=10.4, 3.6 Hz, 1H), 6.14 (d, J=9.6 Hz, 1H), 5.88 (br, 1H), 5.57 (d, J=3.6 Hz, 1H), 5.01 (dd, J=17.8, 1.6 Hz 1H), 4.87 (br, 1H), 4.83 (dd, J=18.0, 1.6 Hz, 1H), 4.66 (q, J=6.4, 1H), 4.04 (br, 1H), 4.00 (d, J=2.4, 1H), 3.99 (dd, J=8.4, 3.6 Hz, 1H), 3.79 (dq, J=9.6, 6.4 Hz, 1H), 3.65 (dd, J=9.6, 9.6 Hz, 1H), 2.79 (m, 1H), 2.16 (m, 2H), 2.00-1.27 (m, 19H), 1.41 (d, J=6.4 Hz, 3H), 1.32 (d, J=6.4 Hz, 3H), 0.95 (s, 3H), 0.88 (s, 3H); ¹³C NMR (400 MHz, CDCl₃) δ 196.6, 174.8 (2C), 143.03, 127.7, 117.9, 97.6, 94.8, 85.8, 80.4, 73.7, 72.4, 72.1, 71.7, 71.1, 68.3, 51.1, 49.8, 42.0, 40.2, 36.7, 35.9, 35.4, 33.4, 30.6, 29.6, 27.1, 26.8, 26.7, 24.0, 21.6, 21.4, 17.8, 16.0, 15.6. ESIHRMS Calcd for [C₃₅H₅₀O₁₀+H]⁺: 631.3482. Found: 631.3487.

### Synthesis of [2,3-didehydro-4-hydroxy-α-L-Rhap-(1→3)-2,4-dihydroxy-α-L-Rhap]-Dig (A-3)

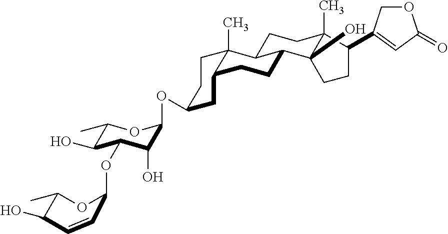

To a CH₂Cl₂ (0.3 mL) solution of enone A-2 (90 mg, 0.14 mmol) in CeCl₃/MeOH (0.4 M in MeOH, 0.3 mL) cooled to −78° C. was added NaBH₄ (8.3 mg, 0.21 mmol) and the resulting solution was stirred at −78° C. for 3 h. The reaction was monitored by TLC and was diluted with EtOAc (5 mL), quenched with 5 mL of saturated aqueous NaHCO₃, extracted with EtOAc (3×5 mL), dried over Na₂SO₄ and concentrated under reduced pressure. The crude product was purified by using silica gel flash chromatography, eluting with 75-80% EtOAc/hexanes to give allylic alcohol A-3 (79 mg, 0.125 mmol, 87%) as a white solid: R_{f} (100% EtOAc)=0.3; mp: 125-130° C.; [α]²⁵_{D}=−25 (c=0.17, CH₂Cl₂); IR (thin film, cm⁻¹) 3427, 2970, 2935, 1776, 1730, 1618, 1448, 1402, 1390, 1166, 1096, 980, 722; ¹H NMR (400 MHz, CDCl₃) δ 6.00 (d, J=8.8 Hz, 1H), 5.88 (br, 1H), 5.82 (ddd, J=10.0, 2.4, 2.0 Hz, 1H), 5.25 (s, 1H), 5.01 (dd, J=18.4, 1.6 Hz, 1H), 4.88 (br, 1H), 4.83 (dd, J=18.4, 1.2 Hz, 1H), 4.02 (dd, J=4.0, 2.4 Hz, 1H), 3.97 (m, 1H), 3.90 (dd, J=9.6, 2.8 Hz, 1H), 3.86 (br, 1H), 3.79 (dq, J=8.8, 6.4 Hz, 1H), 3.76 (dq, J=9.6, 6.0 Hz, 1H), 3.61 (dd, J=9.6, 9.6 Hz, 1H), 2.78 (m, 1H), 2.18 (m, 2H), 2.00-1.27 (m, 19H), 1.34 (d, J=6.4 Hz, 3H), 1.31 (d, J=6.4 Hz, 3H), 0.93 (s, 3H), 0.87 (s, 3H); ¹³C NMR (100 MHz, CDCl₃) δ 174.80 (2C), 134.1, 126.2, 117.9, 97.4, 96.0, 85.8, 80.5, 73.7, 72.1, 71.9, 71.7, 69.4, 68.9, 68.1, 51.1, 49.8, 42.0, 40.2, 36.7, 35.9, 35.4, 33.4, 30.6, 29.6, 27.1, 26.8, 26.7, 24.0, 21.6, 21.4, 18.2, 17.9, 16.0; ESIHRMS Calcd for [C₃₅H₅₂O₁₀+H]⁺: 633.3639. Found: 633.3642.

### Synthesis of [2,3-dihydro-4-hydroxy-α-L-Amip-(1→3)-2,4-dihydroxy-α-L-Rhap]-Dig (A-4-Dihydro)

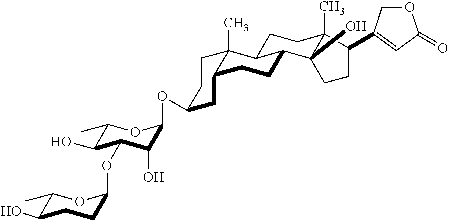

To 4-methylmorpholine (NMM) (0.16 mL, 0.2M) solution of allylic alcohol A-3 (20 mg, 0.0316 mmol) at 0° C. was added o-nitrobenzenesulfonyl hydrazine (NBSH) (35 mg, 0.16 mmol) followed by addition of Et₃N (8.6 μl, 0.063 mmol). The resulting mixture was stirred from 0° C. to rt for 24 h. The reaction mixture was diluted with EtOAc and quenched with saturated NaHCO₃ solution. The mixture was extracted with EtOAc (3×30 ml), dried over Na₂SO₄, and concentrated under reduced pressure. The crude product was purified via silica gel flash chromatography eluting with 80-85% EtOAc/hexanes to give alcohol (A-4-Dihydro) as white solid (18 mg, 0.028 mmol, 90%): R_{f} (100% EtOAc)=0.3; mp: 152-157° C.; [α]²⁵_{D}=−49 (c=0.45, CH₂Cl₂); IR (thin film, cm⁻¹) 3461, 2922, 2868, 1778, 1618; 1460, 1384, 1148, 1120, 1040, 980, 954, 734; ¹H NMR (400 MHz, CDCl₃) δ 5.87 (br, 1H), 5.09 (s, 1H), 5.01 (d, J=18.4 Hz, 1H), 4.87 (s, 1H), 4.83 (d, J=18.4 Hz, 1H), 3.97 (br, 2H), 3.86 (dd, J=9.6, 2.8 Hz, 1H), 3.79 (m, 2H), 3.61 (dd, J=9.6, 8.8 Hz, 1H), 3.33 (m, 1H), 2.78 (m, 1H), 2.19 (m, 2H), 1.92-1.27 (m, 23H), 1.25 (d, J=6.8 Hz, 3H), 1.22 (d, J=6.8 Hz, 3H), 0.93 (s, 3H), 0.87 (s, 3H); ¹³C NMR (100 MHz, CDCl₃) δ 174.7 (2C), 117.9, 98.2, 97.5, 85.8, 79.4, 73.7, 72.4, 71.92, 71.90, 71.6, 70.8, 68.2, 51.1, 49.8, 42.0, 40.2, 36.7, 35.9, 35.4, 33.4, 30.6, 29.9, 29.8, 29.6, 27.6, 27.1, 26.8, 26.7, 24.0, 21.6, 21.4, 18.1, 17.9, 16.0; ESIHRMS Calcd for [C₃₅H₅₄O₁₀+H]⁺: 635.3795. Found: 635.3796.

### Synthesis of [2,3,4-trihydroxy-α-L-Rhap-(1→3)-2,4-dihydroxy-α-L-Rhap]-Dig (A-4)

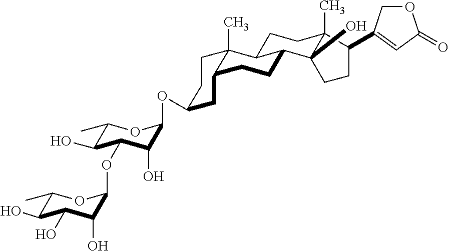

To a t-BuOH/acetone (130 μL, 1:1 (v/v), 0.5M) solution of allylic alcohol A-3 (40 mg, 0.063 mmol) at 0° C. was added a solution of N-methylmorpholine-N-oxide/water (NMO) (50% w/v, 65 μL), followed by addition of OsO₄ (0.81 mg, 5 mol %). The reaction mixture was continued to stir for 8 h. The reaction mixture was quenched with 1 mL of saturated Na₂S₂O₃ solution, extracted with EtOAc (3×10 ml), dried over Na₂SO₄, and concentrated under reduced pressure. The crude product was purified via silica gel flash chromatography eluting with 4-6% MeOH/DCM. Pure fractions were combined, concentrated and recrystallized from EtOH/hexanes to afford A-4, as white solid (39 mg, 0.058 mmol, 92%): R_{f} (10% MeOH/DCM)=0.2; mp=158-162° C.; [α]²⁵_{D}=−55 (c=0.7, MeOH); IR (thin film, cm⁻¹) 3420, 2922, 2870, 1788, 1730, 1630; 1450, 1133, 1090, 1060, 982, 860; 788; ¹H NMR (400 MHz, CD₃OD) δ 5.90 (br, 1H), 5.06 (dd, J=18.4, 1.2 Hz, 1H), 5.02 (d, J=1.2 Hz, 1H), 4.94 (dd, J=18.4, 1.6 Hz, 1H), 4.76 (d, J=1.6 Hz, 1H), 3.98 (dd, J=3.2, 1.6 Hz, 1H), 3.97 (dd, J=3.6, 2.0 Hz, 1H), 3.86 (m, 2H), 3.78 (dd, J=9.6, 2.8 Hz, 1H), 3.76 (m, 1H), 3.71 (dq, J=9.6, 6.0 Hz, 1H), 3.53 (dd, J=9.6, 9.6 Hz, 1H), 3.42 (dd, J=9.6, 9.6 Hz, 1H), 2.86 (m, 1H), 2.23 (m, 2H), 2.00-1.27 (m, 22H), 1.23 (d, J=6.4 Hz, 3H), 0.97 (s, 3H), 0.89 (s, 3H); ¹³C NMR (100 MHz, CD₃OD) δ 178.5, 177.3, 117.8, 103.8, 99.7, 86.5, 78.8, 75.4, 74.0, 73.6, 73.5, 72.9, 72.2, 72.1, 70.4, 70.1, 52.1, 51.1, 42.7, 40.9, 38.2, 36.8, 36.4, 33.4, 31.6, 30.8, 28.1, 27.9, 27.5, 24.4, 22.6, 22.4, 18.0, 17.95, 16.4; ESIHRMS Calcd for [C₃₅H₅₄Oₘ+H]⁺: 667.3694. Found: 637.3694.

### Synthesis of [2,3-didehydro-4-oxo-α-D-Rhap-(1→3)-2,4-dihydroxy-α-L-Rhap]-Dig (A-2-D)

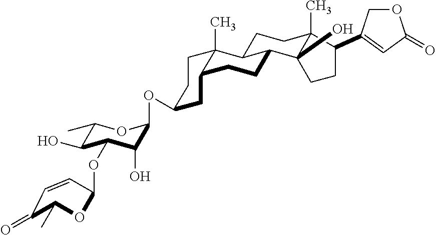

To a solution of digitoxin rhamnose A-1 (60 mg, 0.115 mmol) in CH₃CN/THF (1:0.2) (2.2 mL, 0.05 M) was added boron catalyst (3.9 mg, 0.017 mmol), stirred at 0° C. for 20 min then was added α-D-Boc-pyranone (34 mg, 0.15 mmol) followed by addition of previously mixed solution of Pd₂(dba)₃.CHCl₃ (5.96 mg)/PPh₃ (6.05 mg) in CH₃CN/THF at 0° C. under argon atmosphere. Reaction was continued to stir for 6 h. Quenched with 5 mL of saturated NaHCO₃ solution and extracted with EtOAc, dried over Na₂SO₄ and concentrated under reduced pressure. The crude product was purified using silica gel flash chromatography eluting with 65-70% EtOAc/hexane to give enone (46 mg, 0.073 mmol, 63%); R_{f}(100% EtOAc)=0.5; [α]²⁵_{D}=−32 (c=0.1, CH₂Cl₂); IR (thin film, cm⁻¹) 3354, 2978, 2822, 1718, 1366, 1238, 1180, 1045, 1015, 725, 697; ¹H NMR (400 MHz, CDCl₃) δ 6.87 (dd, J=10.4, 2.8 Hz, 1H), 6.16 (d, J=9.6 Hz, 1H), 5.88 (br, 1H), 5.45 (d, J=3.6 Hz, 1H), 5.01 (d, J=18.4 Hz, 1H), 4.90 (br, 1H), 4.83 (d, J=18.4 Hz, 1H), 4.77 (q, J=7.2 Hz, 1H), 3.98 (m, 2H), 3.91 (dd, J=9.6, 3.6 Hz, 1H), 3.75 (dq, J=9.6, 6.4 Hz, 1H), 3.61 (dd, J=9.6, 9.6 Hz, 1H), 2.80 (m, 1H), 2.16 (m, 2H), 1.87-1.44 (m, 19H), 1.43 (d, J=6.0 Hz, 3H), 1.34 (d, J=6.0 Hz, 3H), 0.95 (s, 3H), 0.88 (s, 3H); ¹³C NMR (100 MHz, CDCl₃) δ 195.8, 174.7 (2C), 142.7, 127.8, 117.9, 97.3, 93.4, 85.8, 82.1, 73.6, 72.0, 71.9, 71.6, 70.8, 68.2, 51.1, 49.8, 42.1, 40.2, 36.7, 35.9, 35.4, 33.4, 30.6, 29.5, 27.1, 26.8, 26.6, 24.0, 21.6, 21.4, 17.9, 16.0, 15.5; ESIHRMS Calcd for [C₃₅H₅₀O₁₀+H]⁺: 631.3482. Found: 631.3489.

### Synthesis of [2,3-didehydro-4-hydroxy-α-D-Rhap-(1→3)-2,4-dihydroxy-α-L-Rhap]-Dig (A-3-D)

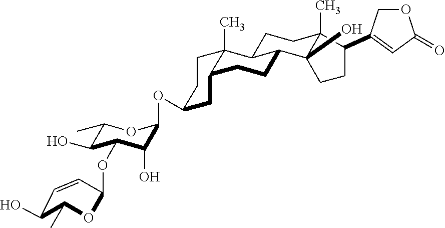

A CH₂Cl₂ (0.9 mL) solution of enone A-2-D (40 mg, 0.063 mmol) in CeCl₃.MeOH (0.4 M in MeOH, 0.15 mL) was cooled to −78° C., to it was added NaBH₄ (3.8 mg, 0.095 mmol) and the resulting solution was stirred at −78° C. for 3 h. The reaction mixture was diluted with EtOAc (5 mL) and was quenched with 4 mL of saturated aqueous NaHCO₃, extracted with EtOAc (3×10 mL), dried over Na₂SO₄, and concentrated under reduced pressure. The crude product was purified by silica gel flash chromatography eluting with 75-80% EtOAc/hexanes to give allylic alcohol (35 mg, 0.055 mmol, 87%) as a white sticky solid; R_{f} (100% EtOAc)=0.3; mp: 125-128° C.; [α]²⁵_{D}=−5.4 (c=0.98, CH₂Cl₂); IR (thin film, cm⁻¹) 3432, 2975, 2925, 1772, 1722, 1611, 1448, 1379, 1170, 1096, 981, 718; ¹H NMR (400 MHz, CDCl₃) δ 6.01 (d, J=9.6 Hz, 1H), 5.87 (br, 1H), 5.77 (dd, J=9.6, 2.4 Hz, 1H), 5.14 (s, 1H), 5.01 (d, J=18.4, 1.6 Hz, 1H), 4.87 (br, 1H), 4.83 (dd, J=18.4, 1.2 Hz, 1H), 4.02 (br, 1H), 3.96 (brs, 1H), 3.88 (dq, J=10.0, 6.4 Hz, 1H), 3.85 (m, 1H), 3.76 (dd, J=9.6, 3.6 Hz, 1H), 3.72 (dq, J=9.6, 6.4 Hz, 1H), 3.54 (dd, J=9.6, 8.8 Hz, 1H), 2.78 (m, 1H), 2.18 (m, 2H), 1.86-1.39 (m, 19H), 1.38 (d, J=5.2 Hz, 3H), 1.34 (d, J=6.0 Hz, 3H), 0.93 (s, 3H), 0.87 (s, 3H); ¹³C NMR (100 MHz, CDCl₃) δ 174.8 (2C), 134.1, 126.0, 117.9, 97.2, 95.2, 85.8, 82.2, 73.7, 71.9, 71.7, 71.3, 69.3, 69.2, 68.1, 51.1, 49.8, 42.0, 40.2, 36.7, 35.9, 35.4, 33.4, 30.6, 29.5, 27.1, 26.8, 26.6, 24.0, 21.6, 21.4, 18.0, 17.9, 16.0; ESIHRMS Calcd for [C₃₅H₅₂O₁₀+H]⁺: 633.3639. Found: 633.3636.

### Synthesis of [2,3,4-trihydroxy-α-D-Rhap-(1→3)-2,4-dihydroxy-α-L-Rhap]-Dig (A-4-D)

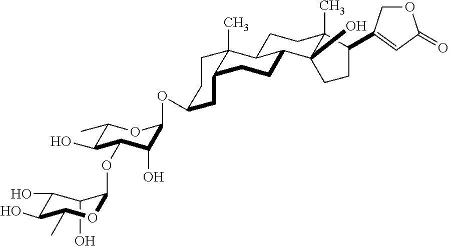

To a solution of allylic alcohol A-3-D (20 mg, 0.032 mmol) in t-BuOH/acetone (130 μl, 1:1 (v/v), 0.5M) at 0° C. was added a solution of N-methylmorpholine-N-oxide/water (NMO) (50% w/v, 33 μl). Crystalline OsO₄ (0.4 mg, 5 mol %) was added and the reaction mixture was stirred for 10 h. The reaction mixture was quenched with 1.5 mL of saturated Na₂S₂O₃ solution, extracted with EtOAc (3×10 ml), dried over Na₂SO₄, and concentrated under reduced pressure. The crude product was purified using silica gel flash chromatography eluting with 4-6% MeOH/DCM to get compound. Pure fractions were combined, concentrated, to afford as solid (18 mg, 0.027 mmol, 85%); R_{f}(10% MeOH/DCM)=0.2; mp=176-180° C.; [α]²⁵_{D}=+21 (c=0.98, CH₂Cl₂); IR (thin film, cm⁻¹) 3406, 2920, 2878, 1766, 1728, 1632, 1455, 1378, 1233, 1118, 1038, 978, 880; 768; ¹H NMR (400 MHz, CD₃OD) δ 5.90 (br, 1H), 5.06 (dd, J=18.4, 1.2 Hz, 1H), 4.94 (d, J=1.6 Hz, 1H), 4.88-4.85 (m, 1H), 4.82 (d, J=1.2 Hz, 1H), 3.96-3.94 (m, 2H), 3.93-3.89 (m, 2H), 3.82 (dd, J=6.8, 2.8 Hz, 1H), 3.80 (m, 1H), 3.73 (dq, J=9.6, 5.6 Hz, 1H), 3.50 (dd, J=9.6, 9.2 Hz, 1H), 3.42 (dd, J=9.6, 9.6 Hz, 1H), 2.84 (m, 1H), 2.21 (m, 2H), 2.01-1.27 (m, 22H), 1.27 (d, J=6.4 Hz, 3H), 0.96 (s, 3H), 0.89 (s, 3H); ¹³C NMR (100 MHz, CD₃OD) δ 178.5, 177.3, 117.8, 99.8, 98.0, 86.5, 76.5, 75.4, 74.0, 73.8, 72.4, 72.3, 72.2, 70.0, 69.9, 68.7, 52.1, 51.1, 42.7, 40.9, 38.2, 36.8, 36.4, 33.4, 31.6, 30.9, 28.1, 27.9, 27.5, 24.3, 22.6, 22.4, 18.1, 18.0, 16.4; ESIHRMS Calcd for [C₃₅H₅₄O₁₂+H]⁺: 667.3694. Found: 667.3687.

### Synthesis of [2,3-dihydro-4-hydroxy-α-D-Amip-(1→3)-2,4-dihydroxy-α-L-Rhap]-Dig (A-4-D-Dihydro)

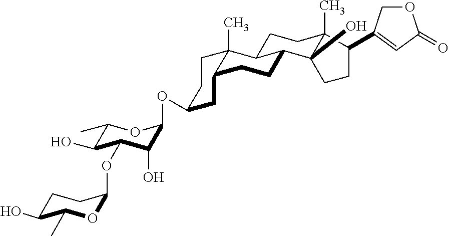

To a N-Methylmorpholine (NMM) (0.16 ml, 0.2M) solution of allylic alcohol (15 mg, 0.024 mmol) at 0° C. was added o-nitrobenzenesulfonyl hydrazine (NBSH) (35 mg, 0.16 mmol) and Et₃N (9 μl mg, 0.03 mmol). The resulting mixture was stirred at 0° C. and gradually raised to rt for 24 h. The reaction mixture was diluted with EtOAc and quenched with saturated NaHCO₃ solution. The mixture was extracted with EtOAc (3×30 ml), dried over Na₂SO₄, and concentrated under reduced pressure, the crude product was purified using silica gel flash chromatography eluting with 80-90% EtOAc/hexanes to give alcohol as white sticky solid, recrystallized with ethanol/hexane (12 mg, 0.019 mmol, 79%); R_{f} (100% EtOAc)=0.3; mp: 160-164° C.; [α]²⁵_{D}=+6.8 (c=0.85, CH₂Cl₂); IR (thin film, cm⁻¹) 3430, 2916, 2838, 1766, 1621; 1448, 1381, 1139, 1125, 1030, 954; 729; ¹H NMR (400 MHz, CDCl₃) δ 5.87 (br, 1H), 5.01 (dd, J=18.4, 1.2 Hz, 1H), 4.98 (s, 1H), 4.8 (d, J=1.6 Hz, 1H), 4.83 (d, J=18.4, 1.6 Hz, 1H). 4.01 (br, 1H), 3.96 (m, 1H), 3.92 (dd, J=3.2, 1.6 Hz, 1H), 3.86 (dq, J=9.6, 6.8, 1H), 3.74 (dd, J=8.8, 2.8 Hz, 1H), 3.70 (m, 1H), 3.54 (dd, J=9.6, 9.6 Hz, 1H), 3.35 (m, 1H), 2.78 (m, 1H), 2.15 (m, 2H), 1.94-1.39 (m, 23H), 1.33 (d, J=6.0 Hz, 3H), 1.31 (d, J=6.4 Hz, 3H), 0.93 (s, 3H), 0.87 (s, 3H); ¹³C NMR (100 MHz, CDCl₃) δ 174.8 (2C), 117.9, 97.2, 96.9, 85.8, 81.3, 73.7, 72.0, 71.7(2C), 71.3, 71.0, 68.0, 51.1, 49.8, 42.0, 40.2, 36.7, 35.9, 35.4, 33.4, 30.6, 30.1, 29.6, 27.4, 27.1, 26.8, 26.6, 23.9, 21.6, 21.4, 18.1, 17.9, 16.0; ESIHRMS Calcd for [C₃₅H₅₄O₁₀+H]⁺: 635.3795. Found: 635.3790.

### Synthesis of [2,3-didehydro-4-oxo-α-L-Rhap-(1→3)-2,4-dihydroxy-α-L-Rhap-(1→3)-2,4-dihydroxy-α-L-Rhap]-Dig (A-5)

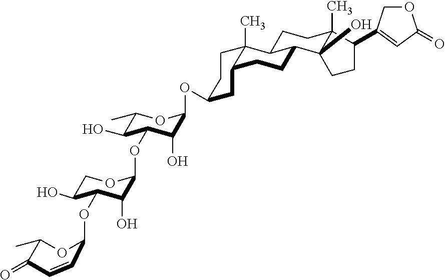

To a solution of disaccharide triol A-4 (35 mg, 0.053 mmol) in CH₃CN/THF (1.05 mL, (1:0.2)) was added boron catalyst at 0° C., stirred for 20 min. To it was added Boc-pyranone (16.0 mg, 0.069 mmol) followed by the addition of Pd₂(dba)₃.CHCl₃ (2.7 mg, 5 mol %) and PPh₃ (2.75 mg, 20 mol %) solution at 0° C. Reaction was continued to stir for 3 h and directly concentrated on rotary evaporator, loaded onto the silica gel column. The crude product was purified by silica gel flash chromatography eluting with 3-4% MeOH/CH₂Cl₂ to give enone A-5 (29 mg, 0.037 mmol, 71%) as a white sticky solid; R_{f} (10% MeOH/CH₂Cl₂)═CH₂Cl₂)=0.65; [α]²⁵_{D}=−45 (c=0.85, CH₂Cl₂); IR (thin film, cm⁻¹) 3480, 2968, 2890, 1753, 1740, 1232, 1190, 1064, 1012, 720; ¹H NMR (400 MHz, CDCl₃) δ 6.92 (dd, J=9.6, 3.6 Hz 1H), 6.14 (d, J=10.4 Hz, 1H), 5.88 (br, 1H), 5.57 (d, J=2.8 Hz, 1H), 5.15 (s, 1H), 5.01 (dd, J=18.4, 1.6 Hz, 1H), 4.85 (s, 1H), 4.83 (dd, J=18.4, 1.2 Hz, 1H), 4.67 (q, J=6.8 Hz, 1H), 4.17 (br, 1H), 3.99 (dd, J=3.6 Hz, 1H), 3.97 (m, 2H), 3.91 (dd, J=8.8, 3.2 Hz, 1H), 3.89 (dd, J=8.8, 3.2 Hz, 1H), 3.75 (m, 1H), 3.68 (dq, J=9.6, 6.4 Hz, 1H), 3.63 (dq, J=9.6, 6.4 Hz, 1H), 2.78 (m, 1H), 2.19 (m, 3H), 1.89 (m, 3H), 1.78-1.49 (m, 12H), 1.42 (d, J=6.8 Hz, 3H), 1.41 (d, J=6.8 Hz, 3H), 1.34 (m, 6H), 0.93 (s, 3H), 0.87 (s, 3H); ¹³C NMR (100 MHz, CDCl₃) δ 196.5, 174.9(2C), 143.0, 127.8, 117.9, 101.4, 97.5, 95.0, 85.8, 80.2, 79.9, 73.7, 72.4, 72.05, 72.02, 71.6, 71.2, 71.05, 69.1, 68.4, 51.1, 49.8, 42.0, 40.2, 36.7, 35.9, 35.4, 33.3, 30.5, 29.9, 29.6, 27.1, 26.8, 26.7, 24.0, 21.6, 21.4, 17.9, 16.0, 15.7; ESIHRMS Calcd for [C₄₁H₆₀O₁₄+H]⁺: 777.4061. Found: 777.4066.

### Synthesis of [2,3-didehydro-4-hydroxy-α-L-Rhap-(1→3)-2,4-dihydroxy-α-L-Rhap-(1→3)-2,4-dihydroxy-α-L-Rhap]-Dig (A-6)

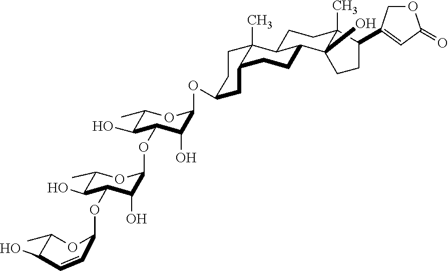

To a stirred solution of enone A-5 (15 mg, 0.019 mmol) in CH₂Cl₂ was added CeCl₃/MeOH solution (0.4 M in MeOH, 0.04 mL) followed by addition of NaBH₄ (1.1 mg, 0.029 mmol) and the resulting solution was stirred at −78° C. for 3 h. The reaction mixture was diluted with EtOAc (5 mL) and was quenched with 0.1 mL of saturated aqueous NaHCO₃, extracted with EtOAc (3×5 mL), dried over Na₂SO₄, and concentrated under reduced pressure. The crude product was purified by silica gel flash chromatography eluting with 5-7% MeOH/CH₂Cl₂ to give allylic alcohol A-6 (12 mg, 0.015 mmol, 80%) as a white solid; R_{f}(10% MeOH/CH₂Cl₂)=0.35; mp: 135-140° C.; [α]²⁵_{D}=−30 (c=0.7, MeOH); IR (thin film, cm⁻¹) 3460, 2968, 2928, 1778, 1730, 1618, 1438, 1400, 1391, 1158, 1096, 980, 730; ¹H NMR (400 MHz, CD₃OD) δ 5.90 (brs, 1H), 5.88 (br, 1H), 5.84 (d, J=2.4 Hz, 1H), 5.21 (s, 1H), 5.05 (s, 1H), 5.02 (d, J=15.6 Hz, 1H), 4.94 (m, 1H), 4.76 (s, 1H), 4.11 (d, J=1.2 Hz, 1H), 3.97 (br, 1H), 3.91 (dd, J=9.6, 2.8 Hz, 1H), 3.86-3.78 (m, 3H), 3.74 (m, 2H), 3.57 (dq, J=9.6, 8.0 Hz, 2H), 2.83 (m, 1H), 2.19 (m, 2H), 1.92 (m, 3H), 1.78-1.46 (m, 13H), 1.27-1.24 (m, 12H), 0.97 (s, 3H), 0.89 (s, 3H); ¹³C NMR (100 MHz, CD₃OD) δ 178.5, 177.3, 134.8, 127.3, 117.8, 103.7, 99.6, 97.9, 86.5, 80.3, 78.6, 75.4, 73.6(2C), 73.1, 72.9, 72.5, 70.5, 70.2 (2C), 69.0, 52.1, 51.1, 42.7, 40.9, 38.3, 36.8, 36.4, 33.4, 31.6, 30.8, 28.1, 27.9, 27.6, 24.4, 22.6, 22.4, 18.3, 18.0, 17.9, 16.4; ESIHRMS Calcd for [C₄₁H₆₂O₁₄+Na]⁺: 801.4037. Found: 801.4045.

### Synthesis of [2,3,4-trihydroxy-α-L-Rhap-(1→3)-2,4-dihydroxy-α-L-Rhap-(1→3)-2,4-dihydroxy-α-L-Rhap]-Dig (A-7)

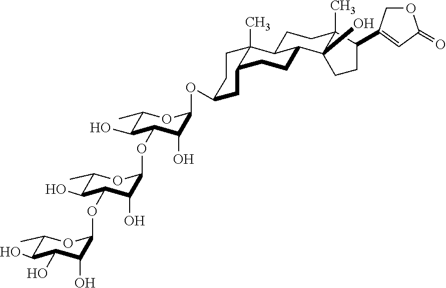

To a t-BuOH/acetone (154 μL, 1:1 (v/v), 0.1M) solution of allylic alcohol A-6 (12 mg, 0.015 mmol) at 0° C. was added a solution of N-methylmorpholine-N-oxide/water (NMO) (50% w/v, 7 μl) followed by addition of crystalline OsO₄ (0.2 mg, 5 mol %). The reaction mixture was stirred for 6 h and quenched with 0.2 mL of saturated Na₂S₂O₃ solution, added silica gel, concentrated on rotary evaporator and directly dry loaded on the column. The crude product was purified via silica gel flash chromatography eluting with 10-12% MeOH/DCM. Pure fractions were combined, concentrated and recrystallized using EtOH/hexanes to afford A-7 as white solid (11 mg, 0.014 mmol, 89%): R_{f} (10% MeOH/DCM)=0.2; mp=185-189° C.; [α]²⁵_{D}=−29 (c=0.4, MeOH); IR (thin film, cm⁻¹) 3443, 2959, 2355, 1733, 1374, 1071; ¹H NMR (500 MHz, CD₃OD) δ 5.93 (s, 1H), 5.09 (dd, J=18.0, 1.5 Hz, 1H), 5.08 (s, 1H), 5.06 (s, 1H), 5.06 (dd, J=18.5, 1.5 Hz, 1H), 4.81 (s, 1H), 4.12 (dd, J=3.5, 2.0 Hz, 1H), 4.03 (dd, J=3.5, 2.0 Hz, 1H), 4.00 (br, 1H), 3.92 (dd, J=9.0, 3.0 Hz, 1H), 3.90 (dd, J=9.5, 3.5 Hz, 1H), 3.89 (m, 2H), 3.83-3.81 (m, 2H), 3.76 (dq, J=10.0, 6.5 Hz, 1H), 3.60 (dd, J=9.5, 9.0 Hz, 1H), 3.56 (dd, J=9.5, 9.0 Hz, 1H), 3.45 (dd, J=9.5, 9.5 Hz, 1H), 2.90 (m, 1H), 2.25 (m, 2H), 1.98-1.48 (m, 16H), 1.33 (m, 12H), 1.01 (s, 3H), 0.93 (s, 3H); ¹³C NMR (100 MHz, CD₃OD) δ 178.5, 177.3, 117.8, 104.1, 103.8, 99.6, 86.5, 80.0, 78.7, 75.4, 74.1, 73.6, 73.5, 73.2, 72.9, 72.2 (2C), 72.0, 70.5, 70.3, 70.1, 52.1, 51.1 42.7, 40.9, 38.3, 36.8, 36.4, 33.4, 31.6, 30.8, 28.1, 27.9, 27.5, 24.4, 22.6, 22.4, 17.98, 17.95 (2C), 16.4; ESIHRMS Calcd for [C₄₁H₆₄O₁₆+Na]⁺: 835.4092. Found: 835.4096.

### Synthesis of [2,3-didehydro-4-oxo-α-D-Rhap-(1→3)-2,4-dihydroxy-α-L-Rhap-(1→3)-2,4-dihydroxy-α-L-Rhap]-Dig (A-5-D)

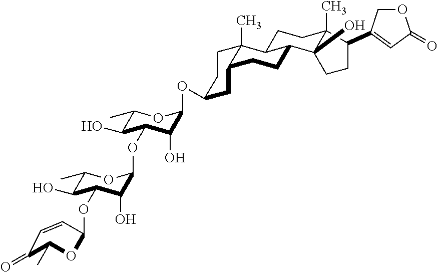

To triol A-4 (32 mg, 0.048 mmol) dissolved in CH₃CN/THF (1:0.2) (1.0 mL) was added boron catalyst (1.6 mg, 15 mol %), stirred for 20 min and then α-D-Boc-pyranone (14.3 mg, 0.062 mmol) was added at 0° C., followed by addition of previously mixed solution of Pd₂(dba)₃.CHCl₃ (2.5 mg)/PPh₃ (2.51 mg) in CH₃CN/THF. Reaction continued to stir for 8 h. The reaction mixture was diluted with EtOAc (5 mL) and was quenched with 5 mL of saturated NaHCO₃ solution, extracted with EtOAc (3×5 mL), dried over Na₂SO₄, and concentrated under reduced pressure. The crude product was purified by silica gel flash chromatography eluting with 4-5% MeOH/CH₂Cl₂ to give trisaccharide enone A-5-D (31 mg, 0.04 mmol, 83%) as white solid: R_{f} (10% MeOH/CH₂Cl₂)=0.6; mp: 145-148° C.; [α]²⁵_{D}=−44 (c=1.5, CH₂Cl₂); IR (thin film, cm⁻¹) 3426, 2933, 2344, 2320, 1717, 1099; ¹H NMR (400 MHz, CDCl₃) δ 6.88 (dd, J=10.4, 2.8 Hz 1H), 6.15 (d, J=10.0 Hz, 1H), 5.88 (brs, 1H), 5.44 (d, J=3.6 Hz, 1H), 5.19 (s, 1H), 5.01 (d, J=18.0 Hz, 1H), 4.84 (s, 1H), 4.83 (d, J=18.4 Hz, 1H), 4.76 (q, J=6.8 Hz, 1H), 4.14 (br, 1H), 3.95 (brs, 1H), 3.93 (d, J=2.8 Hz, 1H), 3.91-3.88 (m, 2H), 3.85 (dq, J=9.0, 6.4 Hz, 1H), 3.73 (dq, J=9.0, 6.4 Hz, 1H), 3.62 (m, 2H), 3.53 (d, J=2.4 Hz, 1H), 2.77 (m, 1H), 2.62 (br, 1H), 2.47 (br, 1H), 2.36 (br, 1H), 2.15 (m, 3H), 1.86 (m, 3H), 1.71-24 (m, 24H), 0.93 (s, 3H), 0.86 (s, 3H); ¹³C NMR (100 MHz, CDCl₃) δ 196.0, 175.1, 175.0, 142.8, 127.7, 117.8, 101.1, 97.5, 93.0, 85.8, 80.8, 79.6, 73.8, 72.5, 72.0, 71.61, 71.58, 71.54, 69.8, 69.0, 68.4, 51.0, 49.8, 42.0, 40.2, 36.7, 35.9, 35.4, 33.3, 30.5, 29.6, 27.1, 26.74, 26.7, 24.0, 21.6, 21.4, 17.9, 17.8, 16.0, 15.5; ESIHRMS Calcd for [C₄H₆₀O₁₄+H]⁺: 777.4061. Found: 777.4067.

### Synthesis of [2,3-didehydro-4-hydroxy-α-D-Rhap-(1→3)-2,4-dihydroxy-α-L-Rhap-(1→3)-2,4-dihydroxy-α-L-Rhap]-Dig (A-6-D)

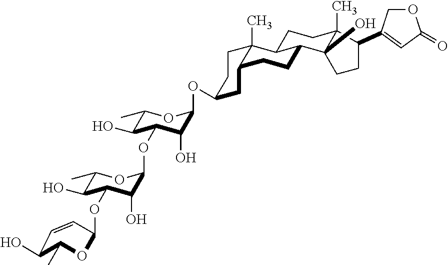

To a CH₂Cl₂ (0.3 mL) solution of enone A-5-D (25 mg, 0.032 mmol) in CeCl₃/MeOH solution (0.4 M in MeOH, 0.06 mL) was cooled to −78° C. and added NaBH₄ (2.0 mg, 0.05 mmol) and the resulting solution was stirred at −78° C. for 3 h. The reaction mixture was diluted with EtOAc (5 mL) and was quenched with 1 mL of saturated aqueous NaHCO₃, extracted with EtOAc (3×5 mL), dried over Na₂SO₄ and concentrated under reduced pressure. The crude product was purified by silica gel flash chromatography eluting with 5-7% MeOH/CH₂Cl₂ to obtain allylic alcohol A-6-D (22 mg, 0.028 mmol, 88%) as white solid: R_{f} (10% MeOH/CH₂Cl₂)=0.35; mp: 172-175° C.; [α]²⁵_{D}=−23 (c=0.45, MeOH); IR (thin film, cm⁻¹) 3466, 2955, 2916, 1770, 1732, 1620, 1438, 1391, 1148, 1087, 978, 722; ¹H NMR (400 MHz, CD₃OD) δ 5.93 (d, J=10.4 Hz, 1H), 5.90 (br, 1H), 5.80 (d, J=10.0 Hz, 1H), 5.12 (s, 1H), 5.08 (s, 1H), 5.06 (d, J=19.2 Hz, 1H), 4.94 (d, J=18.4 Hz, 1H), 4.76 (s, 1H), 4.12 (br, 1H), 3.96 (dd, J=9.6, 2.8 Hz, 1H), 3.92 (dd, J=9.6, 2.8 Hz, 1H), 3.86-3.81 (m, 4H), 3.75 (dq, J=9.6, 6.8 Hz, 1H), 3.69 (dq, J=9.6, 6.8 Hz, 1H), 3.57 (m, 2H), 2.84 (m, 1H), 2.19 (m, 2H), 1.87-1.46 (m, 16H), 1.29-1.24 (m, 12H), 0.97 (s, 3H), 0.89 (s, 3H); ¹³C NMR (100 MHz, CD₃OD) δ 178.5, 177.3, 135.0, 127.3, 117.8, 103.7, 99.6, 93.3, 86.4, 79.0, 78.4, 75.4, 73.6, 73.5, 72.8, 72.3, 70.4, 70.1 (2C), 69.2 (2C), 52.1, 51.1, 42.7, 40.9, 38.2, 36.8, 36.4, 33.4, 31.6, 30.8, 28.1, 27.9, 27.5, 24.4, 22.6, 22.4, 18.2, 18.1, 17.9, 16.4; ESIHRMS Calcd for [C₄₁H₆₂O₁₄+Na]⁺: 801.4037. Found: 801.4037.

### Synthesis of [2,3,4-trihydroxy-α-D-Rhap-(1→3)-2,4-dihydroxy-α-L-Rhap-(1→3)-2,4-dihydroxy-α-L-Rhap]-Dig (A-7-D)

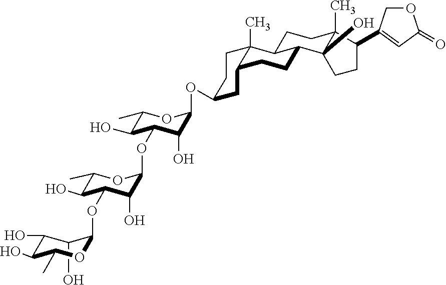

To a t-BuOH/acetone (260 μL, 1:1 (v/v), 0.1M) solution of allylic alcohol A-6-D (20 mg, 0.026 mmol) at 0° C. was added a solution of N-methylmorpholine-N-oxide/water (50% w/v, 12 μL). Crystalline OsO₄ (0.33 mg, 5 mol %) was added and the reaction mixture was stirred for 6-8 hours. The reaction mixture was quenched with 0.1 mL of saturated Na₂S₂O₃ solution and added silica gel, concentrated on rotary evaporator and directly loaded on the column. The crude product was purified via silica gel flash chromatography eluting with 10-13% MeOH/DCM. Pure fraction were combined, concentrated, crystallized using EtOH/hexane/ether to afford A-7-D as solid (16 mg, 0.0197 mmol, 76%); R_{f} (10% MeOH/DCM)=0.2; mp=138-145° C.; [α]²⁵_{D}=−12 (c=0.44, MeOH); IR (thin film, cm⁻¹); 3388, 2929, 2871, 2341, 1738, 1380, 1048, 982; ¹H NMR (500 MHz, CD₃OD) δ 5.95 (br, 1H), 5.13 (d, J=1.5 Hz, 1H), 5.10 (dd, J=18.5, 1.5 Hz, 1H), 4.99 (dd, J=18.5, 1.5 Hz, 1H), 4.94 (d, J=1.5 Hz, 1H), 4.81 (d, J=1.5 Hz, 1H), 4.20 (dd, J=3.0, 1.5 Hz, 1H), 4.01 (br, 1H), 3.99 (m, 1H), 3.97 (dd, J=3.5, 1.5 Hz, 1H), 3.94 (dd, J=10.0, 2.5 Hz, 1H), 3.90 (m, 2H), 3.88 (m, 1H), 3.85 (dd, J=10.0, 3.5 Hz, 1H), 3.76 (dq, J=10.0, 6.5 Hz, 1H), 3.58-3.52 (m, 3H), 3.47 (dd, J=10.0, 9.5 Hz, 1H), 2.90 (m, 1H), 2.25 (m, 2H), 1.97-1.49 (m, 16H), 1.33 (m, 13H), 1.24 (d, J=7.5 Hz, 1H), 1.23 (d, J=7.0 Hz, 1H), 1.02 (s, 3H), 0.94 (s, 3H); ¹³C NMR (100 MHz, CD₃OD) δ 178.5, 177.3, 117.8, 103.8, 99.7, 98.3, 86.5, 79.3, 76.8, 75.4, 74.0, 73.6, 73.4, 72.8, 72.4, 72.2 (2C), 70.4, 70.0, 69.9, 68.3, 52.1, 51.1 42.7, 40.9, 38.2, 36.8, 36.4, 33.4, 31.6, 30.8, 28.1, 27.9, 27.5, 24.4, 22.6, 22.4, 18.1, 17.9 (2C), 16.4; ESIHRMS Calcd for [C₄₁H₆₄O₁₆+Na]⁺: 835.4092. Found: 835.4101.

### Synthesis of 2,3-didehydro-4-oxo-α-L-Rhap-(1→3)-2,4-dihydroxy-α-L-Rhap-(1→3)-2,4-dihydroxy-α-L-Rhap-(1→3)-2,4-dihydroxy-α-L-Rhap]-Dig (A-8)

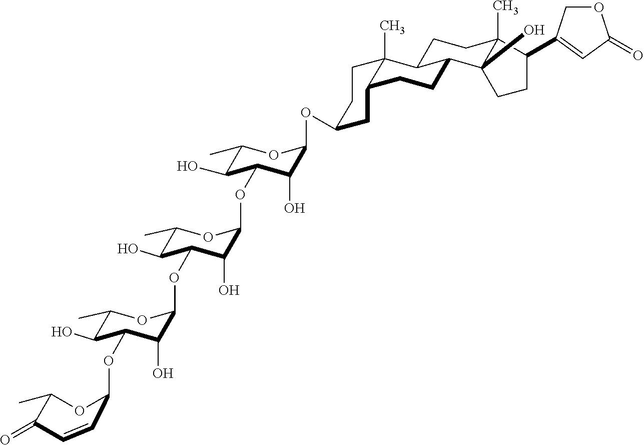

To a stirred solution of trisaccharide triol A-7 (30 mg, 0.037 mmol) in CH₃CN/THF (0.74 mL (1:0.2)) was added boron catalyst (1.3 mg, 15 mol %), stirred for 20 min and was added α-L-Boc-pyranone (11.0 mg, 0.048 mmol) at 0° C., followed by addition of Pd₂(dba)₃.CHCl₃ (1.91 mg)/PPh₃ (1.94 mg) solution in CH₃CN/THF. Reaction was continued to stir for 6 h. The reaction mixture was concentrated under reduced pressure and directly loaded on the column. The crude product was purified using silica gel flash chromatography eluting with 6-8% MeOH/CH₂Cl₂ to give enone A-8 (24.8 mg, 0.027 mmol, 73%) as a sticky oil: R_{f}(10% MeOH/CH₂Cl₂)=0.3; [α]²⁵_{D}=−69 (c=0.67, MeOH); IR (thin film, cm⁻¹) 3425, 2960, 2343, 2319, 1761, 1732, 1041, 908; ¹H NMR (400 MHz, CD₃OD) δ 7.08 (dd, J=10.4, 3.6 Hz, 1H), 6.08 (d, J=10.0 Hz, 1H), 5.92 (br, 1H), 5.54 (s, 1H), 5.07 (s, 1H), 5.05 (s, 1H), 5.03 (s, 1H), 4.96 (m, 1H), 4.81 (m, 2H), 4.20 (br, 1H), 4.10 (br, 1H), 4.03 (d, J=8.0 Hz, 1H), 3.98 (br, 1H), 3.91 (dd, J=9.6, 9.6 Hz, 1H), 3.89 (dd, J=9.6, 9.6 Hz, 1H), 3.86 (br, 2H), 3.81 (m, 1H), 3.71 (m, 1H), 3.57 (m, 3H), 2.86 (m, 1H), 2.22 (m, 2H), 1.92-1.48 (m, 16H), 1.35 (d, J=6.4 Hz, 4H), 1.31-1.25 (m, 11H), 0.98 (s, 3H), 0.90 (s, 3H); ¹³C NMR (100 MHz, CD₃OD) δ 199.2, 178.5, 177.3, 145.8, 127.6, 117.8, 104.1, 103.8, 99.6, 96.6, 86.4, 81.0, 80.0, 78.8, 75.4, 73.6, 73.5, 73.2 (2C), 72.9, 72.0, 71.9, 71.6, 70.5, 70.34, 70.27, 52.1, 51.1, 42.7, 40.9, 38.3, 36.8, 36.4, 33.4, 31.6, 30.8, 28.1, 27.9, 27.6, 24.4, 22.6, 22.4, 17.9, 16.4, 15.5.

### Synthesis of [2,3-didehydro-4-hydroxy-α-L-Rhap-(1→3)-2,4-dihydroxy-α-L-Rhap-(1→3)-2,4-dihydroxy-α-L-Rhap-(1→3)-2,4-dihydroxy-α-L-Rhap]-Dig (A-9)

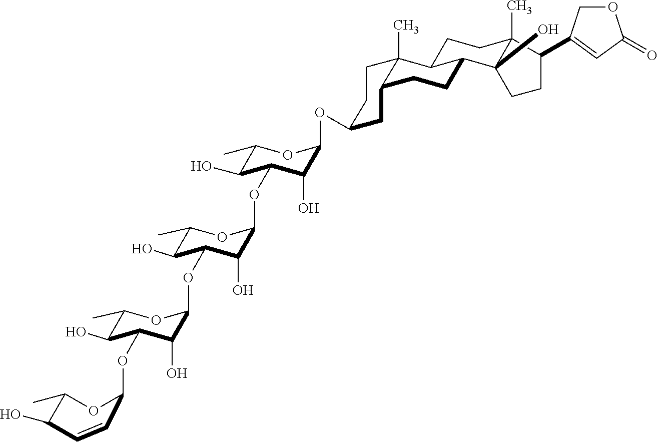

To a stirred solution of enone A-8 (20 mg, 0.022 mmol) in CH₂Cl₂ was added CeCl₃/MeOH solution (0.4 M in MeOH, 0.04 mL) at −78° C. followed by addition of NaBH₄ (1.3 mg, 0.033 mmol). The resulting solution was stirred at −78° C. for 3 h. The reaction mixture was diluted with EtOAc (5 mL) and was quenched with 0.1 mL of saturated NaHCO₃ solution, added silica gel and concentrated under reduced pressure. The crude product was purified using silica gel flash chromatography, eluting with 6-10% MeOH/DCM to give tetra-saccharide allylic alcohol A-9 (15 mg, 0.016 mmol, 74%) as sticky oil; R_{f} (10% MeOH/DCM)=0.15; [α]²⁵_{D}=−66 (c=1.38, MeOH); IR (thin film, cm⁻¹) 3377, 2932, 1736, 1641, 1449, 1401, 1044, 989, 832; ¹H NMR (400 MHz, CD₃OD) δ 5.90 (br 1H), 5.88-5.82 (m, 2H), 5.22 (s, 1H), 5.05 (s, 1H), 5.03 (s, 1H), 5.02 (br, 1H), 4.94 (d, J=18.4 Hz, 1H), 4.76 (s, 1H), 4.12 (br, 1H), 4.08 (br, 1H), 3.96 (brs, 1H), 3.93-3.79 (m, 6H), 3.81 (m, 2H), 3.57 (dd, J=9.6, 4.0 Hz, 1H), 3.52 (m, 3H), 2.84 (m, 1H), 2.21 (m, 2H), 1.92-1.46 (m, 16H), 1.31-1.25 (m, 15H), 0.97 (s, 3H), 0.89 (s, 3H); ¹³C NMR (100 MHz, CD₃OD) δ 178.5, 177.3, 134.7, 127.3, 117.8, 103.9, 103.7, 99.6, 97.8, 86.4, 80.2, 79.7, 78.7, 75.4, 73.6, 73.5, 73.2 (2C), 72.8, 72.5, 72.0, 70.4, 70.3 (2C), 70.2, 68.9, 52.1, 51.1, 42.7, 40.9, 38.2, 36.8, 36.4, 33.4, 31.6, 30.8, 28.1, 27.9, 27.5, 24.4, 22.6, 22.4, 18.3, 17.98, 17.95, 16.4; ESIHRMS Calcd for [C₄₇H₇₂O₁₈+Na]⁺: 947.4616. Found: 947.4617.

### Synthesis of [2,3,4-trihydroxy-α-L-Rhap-(1→3)-2,4-dihydroxy-α-L-Rhap-(1→3)-2,4-dihydroxy-α-L-Rhap-(1→3)-2,4-dihydroxy-α-L-Rhap]-Dig (A-10)

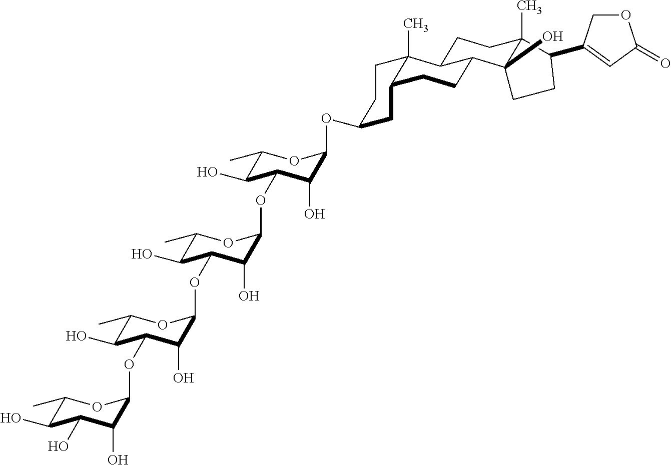

To a t-BuOH/acetone (110 μL, 1:1 (v/v), 0.1M) solution of allylic alcohol A-9 (11 mg, 0.012 mmol) at 0° C. was added a solution of NMO (50% w/v, 5 μL) followed by addition of OsO₄ (0.15 mg, 5 mol %) and the reaction mixture was stirred for 8 h. The reaction mixture was quenched with 0.1 mL of saturated Na₂S₂O₃ solution and directly concentrated under reduced pressure and loaded on the column. The crude product was purified via silica gel flash chromatography eluting with 15-18% MeOH/DCM, pure fractions were combined, concentrated and recrystallized from EtOH/hexanes to afford A-10 as white solid (9 mg, 0.009 mmol, 78%): R_{f} (20% MeOH/DCM)=0.2; [α]²⁵_{D}=−85 (c=0.55, MeOH); mp: 194-198° C.; IR (thin film, cm⁻¹) 3419, 2922, 2344, 1746, 1115, 1071, 1048; ¹H NMR (500 MHz, CD₃OD) δ 5.95 (brs, 1H), 5.11 (d, J=17.5 Hz, 1H), 5.09 (s, 1H), 5.07 (s, 2H), 4.99 (dd, J=18.5, 1.5 Hz, 1H), 4.81 (s, 1H), 4.13 (m, 2H), 4.04 (dd, J=3.5, 1.5 Hz, 1H), 4.01 (br, 1H), 3.94 (dd, J=3.0, 2.0 Hz, 1H), 3.91 (m, 4H), 3.87 (m, 1H), 3.84 (dd, J=10.0, 3.5 Hz, 2H), 3.81 (dd, J=10.0, 3.5 Hz, 1H), 3.76 (dq, J=9.5, 6.5 Hz, 1H), 3.61 (dd, J=9.0, 2.5 Hz, 1H), 3.60 (dd, J=9.0, 2.5 Hz, 1H), 3.59 (dd, J=9.5, 9.5 Hz, 1H), 3.46 (dd, J=10.0, 9.0 Hz, 1H), 2.90 (m, 1H), 2.25 (m, 2H), 1.98-1.50 (m, 16H), 1.33 (m, 15H), 1.02 (s, 3H), 0.94 (s, 3H); ¹³C NMR (100 MHz, CD₃OD): δ 178.5, 177.3, 117.8, 104.1, 104.0, 103.7, 99.6, 86.4, 79.9, 79.9, 78.7, 75.4, 74.1, 73.6, 73.5, 73.3, 73.2, 72.9, 72.2, 72.0 (2C), 70.5, 70.3, 70.1, 52.1, 51.1, 42.7, 41.0, 38.3, 36.8, 36.4, 33.4, 31.6, 30.8, 28.1, 27.9, 27.6, 24.4, 22.6, 22.4, 17.9, 16.4; ESIHRMS Calcd for [C₄₇H₇₄O₂₀+Na]⁺: 981.4671. Found: 981.4673.

### Synthesis of [2,3-didehydro-4-oxo-α-D-Rhap-(1→3)-2,4-dihydroxy-α-L-Rhap-(1→3)-2,4-dihydroxy-α-L-Rhap-(1→3)-2,4-dihydroxy-α-L-Rhap]-Dig (A-8-D)

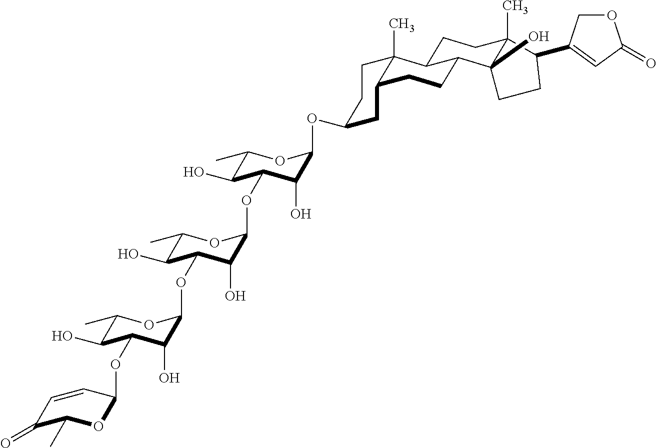

To triol A-7 (22 mg, 0.027 mmol) was dissolved in CH₃CN/THF (0.54 mL (1:0.3)) added boron catalyst (0.9 mg, 15 mol %), stirred for 20 min and then was added α-D Boc-pyranone (8.0 mg, 0.035 mmol) at 0° C., followed by addition of Pd₂(dba)₃.CHCl₃ (1.4 mg) and PPh₃ (1.45 mg, 20 mol %) solution. Reaction was continued to stir for 4 h at 0° C. The reaction mixture was concentrated under reduced pressure and directly loaded on the column. The crude product was purified by silica gel flash chromatography eluting with 5-6% MeOH/CH₂Cl₂ to give enone A-8-D (16 mg, 0.017 mmol, 64%) as a thick oily compound; R_{f} (10% MeOH/CH₂Cl₂)=0.3; [α]²⁵_{D}=−52 (c=0.54, MeOH); ¹H NMR (400 MHz, CD₃OD) δ 7.05 (dd, J=10.4, 3.6 Hz, 1H), 6.08 (d, J=10.4 Hz, 1H), 5.91 (s, 1H), 5.42 (d, J=3.6 Hz, 1H), 5.09 (s, 1H), 5.06 (s, 1H), 5.03 (d, J=17.6 Hz, 1H), 4.95 (br, 1H), 4.77 (s, 1H), 4.22 (br, 1H), 4.11 (br, 1H), 4.10 (dd, J=9.6, 2.8 Hz, 1H), 3.98 (br, 1H), 3.91 (dd, J=10.4, 3.6 Hz, 1H), 3.88 (d, J=10.4 Hz, 1H), 3.86 (br, 2H), 3.84 (m, 1H), 3.73 (m, 1H), 3.61 (m, 3H), 2.85 (m, 1H), 2.22 (m, 2H), 1.92-1.39 (m, 16H), 1.32 (m, 15H), 0.98 (s, 3H), 0.90 (s, 3H); ¹³C NMR (100 MHz, CD₃OD) δ 199.5, 178.5, 177.3, 146.2, 127.8, 117.8, 103.98, 103.81, 99.6, 91.2, 86.5, 80.3, 78.8, 77.7, 75.4, 73.6, 73.5, 73.1, 72.9, 72.4, 71.9, 71.6, 70.5, 70.3, 70.2, 68.5, 52.1, 51.1, 42.7, 40.9, 38.3, 36.8, 36.4, 33.4, 31.6, 30.8, 28.1, 27.9, 27.5, 24.4, 22.6, 22.4, 18.1, 18.0, 17.9, 16.4, 15.5.

### Synthesis of [2,3-didehydro-4-hydroxy-α-L-Rhap-(1→3)-2,4-dihydroxy-α-L-Rhap-(1→3)-2,4-dihydroxy-α-L-Rhap-(1→3)-2,4-dihydroxy-α-L-Rhap]-Dig (A-9-D)

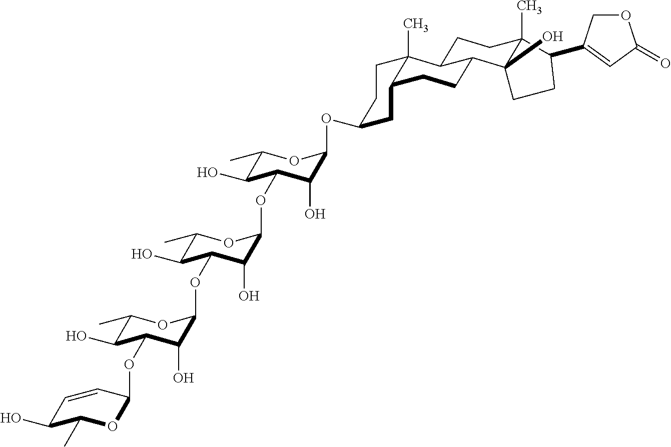

To a solution of enone A-8-D (15 mg, 0.016 mmol) in CH₂Cl₂ (0.16 mL) was added CeCl₃/MeOH solution (0.4 M in MeOH, 0.03 mL) was cooled to −78° C. To it was added NaBH₄ (1.0 mg, 0.024 mmol) and the resulting solution was stirred at −78° C. for 3 h. The reaction mixture was diluted with EtOAc (2 mL) and was quenched with 0.1 mL of saturated aqueous NaHCO₃, added silica gel and concentrated under reduced pressure, directly loaded on the column. The crude product was purified by silica gel flash chromatography eluting with 8-10% MeOH/DCM to give alcohol A-9-D (13 mg, 0.014 mmol, 88%) as a sticky oil: R_{f}(10% MeOH/DCM)=0.15; [α]²⁵_{D}=−28 (c=0.18, MeOH); IR (thin film, cm⁻¹) 3400, 2928, 1734, 1449, 1380, 1043, 737; ¹H NMR (500 MHz, CD₃OD) δ 5.97 (m, 2H), 5.85 (ddd, J=9.5, 2.5, 1.5 Hz, 1H), 5.17 (s, 1H), 5.10 (d, J=17.5 Hz, 1H), 5.09 (s, 2H), 4.98 (d, J=18.5 Hz, 1H), 4.81 (s, 1H), 4.18 (br, 1H), 4.14 (d, J=2.0 Hz, 1H), 4.01 (dd, J=9.5, 3.0 Hz, 1H), 3.99 (dd, J=9.5, 3.0 Hz, 1H), 3.94 (m, 3H), 3.89 (br, 1H), 3.86 (dq, J=9.0, 6.5 Hz, 1H), 3.79 (d, J=9.5 Hz, 1H), 3.76 (dq, J=9.5, 6.0 Hz, 1H), 3.68 (m, 1H), 3.61-3.51 (m, 3H), 2.90 (m, 1H), 2.25 (m, 2H), 1.96-1.50 (m, 16H), 1.35-1.29 (m, 13H), 1.24 (t, J=6.4 Hz, 2H), 1.01 (s, 3H), 0.93 (s, 3H); ¹³C NMR (100 MHz, CD₃OD) δ 178.5, 177.3, 135.0, 127.3, 117.8, 103.96, 103.77, 99.6, 93.3, 86.4, 80.2, 78.7, 78.3, 75.4, 73.6, 73.5, 73.1, 72.9, 72.4, 71.9, 70.5, 70.3, 70.14, 70.07, 69.2, 52.1, 51.1, 42.7, 40.9, 38.2, 36.8, 36.4, 33.4, 31.6, 30.8 (2C), 28.1, 27.9, 27.5, 24.4, 22.6, 22.4, 18.2, 18.1, 18.0, 17.9, 16.4; ESIHRMS Calcd for [C₄₇H₇₂O₁₈+Na]⁺: 947.4611. Found: 947.4625.

### Synthesis of [2,3,4-trihydroxy-α-D-Rhap-(1→3)-2,4-dihydroxy-α-L-Rhap-(1→3)-2,4-dihydroxy-α-L-Rhap-(1→3)-2,4-dihydroxy-α-L-Rhap]-Dig (A-10-D)

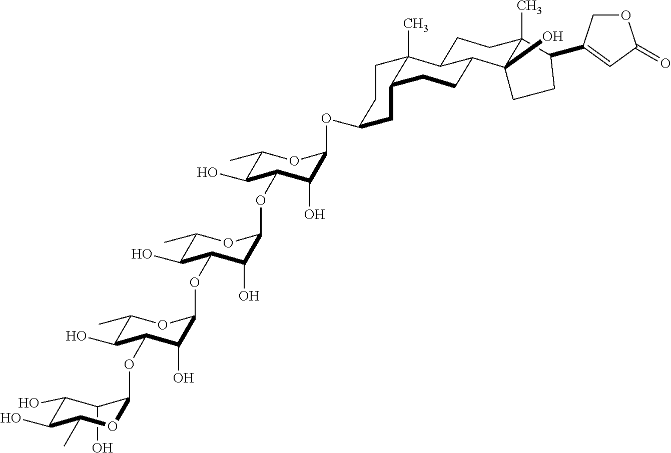

To a t-BuOH/acetone (110 μL, 1:1 (v/v), 0.1M) solution of alcohol A-9-D (10 mg, 0.011 mmol) at 0° C. was added a solution of N-methylmorpholine-N-oxide/water (50% w/v, 4 μl). Crystalline OsO₄ (0.15 mg, 5 mol %) was added and the reaction mixture was stirred for 12 h. The reaction mixture was quenched with 0.1 mL of saturated Na₂S₂O₃ solution, directly concentrated under reduced pressure and dry loaded on the column using silica gel. The crude product was purified via silica gel flash chromatography eluting with 15-18% MeOH/DCM, pure fractions were combined, concentrated, and recrystallized from EtOH/hexanes/ether to afford A-10-D as white solid (8.9 mg, 0.009 mmol, 85%); R_{f} (20% MeOH/DCM)=0.2; [α]²⁵_{D}=−26 (c=0.48, MeOH); mp: 218-225° C.; IR (thin film, cm⁻¹) 3446, 2344, 1734, 1456, 1383, 1046, 988; ¹H NMR (500 MHz, CD₃OD): δ 5.95 (brs, 1H), 5.10 (dd, J=18.0, 1.5 Hz, 1H), 5.10 (m, 2H), 4.99 (dd, J=18.0, 1.5 Hz, 1H), 4.94 (d, J=1.5 Hz, 1H), 4.81 (s, 1H), 4.21 (dd, J=3.0, 2.0 Hz, 1H), 4.14 (dd, J=3.0, 2.0 Hz, 1H), 4.01 (brs, 1H), 4.00 (m, 1H), 3.97 (dd, J=3.5, 2.0 Hz, 1H), 3.96 (dd, J=3.5, 2.0 Hz, 1H), 3.93-3.91 (m, 3H), 3.89 (br, 2H), 3.85 (dd, J=9.5, 3.0 Hz, 1H), 3.83 (dd, J=10.0, 3.5 Hz, 1H), 3.76 (dq, J=9.5, 6.0 Hz, 1H), 3.60 (m, 2H), 3.55 (dd, J=10.0, 9.0 Hz, 1H), 3.47 (dd, J=10.0, 9.0 Hz, 1H), 2.90 (m, 1H), 2.25 (m, 2H), 1.97-1.49 (m, 16H), 1.34 (m, 14H), 1.02 (s, 3H), 0.93 (s, 3H); ¹³C NMR (100 MHz, CD₃OD) δ 178.5, 177.3, 117.8, 104.0, 103.8, 99.6, 98.3, 86.4, 80.4, 78.7, 76.6, 75.4, 74.0, 73.6, 73.5, 73.1, 72.9, 72.4, 72.3, 72.2, 71.9, 70.5, 70.3, 70.1, 69.9, 68.3, 52.1, 51.1, 42.7, 40.9, 38.2, 36.8, 36.4, 33.4, 31.6, 30.8, 28.1, 27.9, 27.5, 24.4, 22.6, 22.4, 18.1, 18.0, 17.9 (2C), 16.4; ESIHRMS Calcd for [C₄₂H₂₄O₂₀+Na]⁺: 981.4666. Found: 981.4639.

### Example 6

### Synthesis of 4-Aminosugar Analogues

The 4-aminosugar compounds were synthesized using the procedure depicted in Scheme 6:

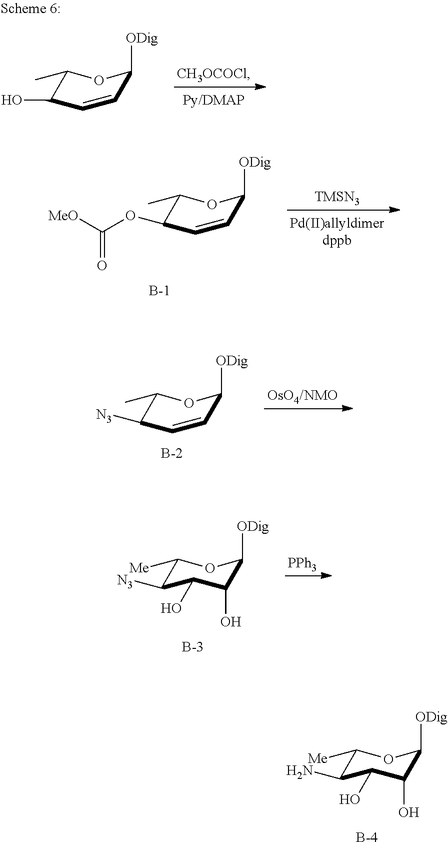

### Synthesis of 2,3-didehydro-4-oxo-carbonato-α-L-Rhap]-Dig (B-1)

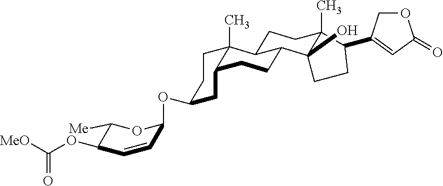

To digitoxin monosaccharide allylic alcohol (94 mg, 0.193 mmol) in CH₂Cl₂ (0.5 mL) added pyridine (80 μL, 0.97 mmol), DMAP (4.8 mg, 0.39 mmol) at 0° C. followed by addition of methyl chloroformate (75 μL, mmol). Reaction continued to stir from 0° C. to rt for 3 h. Reaction diluted with EtOAc and washed with dilute 0.5N HCl. Performed column chromatography with 30-35% EtOAc/hexane to give desired carbonate B-1 (95 mg, 0.174 mmol 90%); R_{f} (50% EtOAc/hexanes)=0.55; [α]²⁵_{D}=−27.2 (c=0.37, CH₂Cl₂); IR (thin film, cm⁻¹) 3497, 2936, 17460, 1621, 1443, 1260, 1067, 1017, 990, 734; ¹H NMR (400 MHz, CDCl₃): δ 5.91 (d, J=10.4 Hz, 1H), 5.87 (br 1H), 5.81 (d, J=9.6 Hz, 1H), 5.04 (s, 1H), 5.02 (d, J=18.4 Hz, 1H), 4.88 (d, J=9.2 Hz, 1H), 4.06 (dq, J=6.0, 2.8 Hz, 1H), 3.97 (brs, 1H), 3.81 (s, 3H), 2.80 (dd, J=8.8, 5.2 Hz, 1H), 2.20-2.09 (m, 2H), 1.88-1.36 (m, 16H), 1.25 (d, J=6.8 Hz, 6H) 0.93 (s, 3H), 0.87 (s, 3H); ¹³C NMR (100 MHz, CDCl₃) δ 174.8, 174.7, 155.6, 129.3, 128.7, 117.9, 93.6, 85.8, 74.9, 74.2, 73.6, 64.8, 55.2, 51.1, 49.8, 42.1, 40.2, 36.6, 35.9, 35.4, 33.4, 31.1, 30.5, 27.1, 27.0, 26.8, 23.9, 21.6, 21.4, 18.1, 16.0.

### Synthesis of [2,3-didehydro-4-azido-α-L-Rhap]-Dig (B-2)

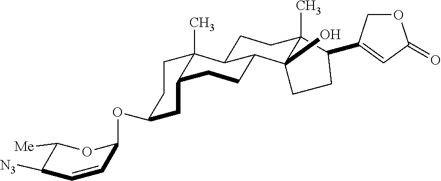

To digitoxin carbonate B-1 (105 mg, 0.193 mmol) dissolved in CH₂Cl₂ (1.5 ml) added 1,4-bisdiphenylphosphinobutane (dppb) (33 mg, 0.0772 mmol), allyl palladium(II)chloride dimer (7.4 mg, 0.0193 mmol) followed by addition of azido(trimethyl)silane (130 μL, 0.965 mmol). Reaction continued to stir at rt for 3 h. Reaction completed, diluted with CH₂Cl₂ and directly loaded on column with elution 30-35% EtOAc/hexane to give allylic azide B-2 (87 mg, 0.170 mmol, 88%); R_{f} (50% EtOAc/hexanes)=0.52; mp: 90-95° C.; [α]²⁵_{D}=−36.5; IR (thin film, cm⁻¹) 3411, 2708, 2493, 2111, 1712, 1536, 1462, 1370, 1350, 1237, 1209, 1187, 1114, 1025, 745, 614; ¹H NMR (400 MHz, CDCl3): δ 5.91 (d, J=12.8 Hz, 1H), 5.88 (br 1H), 5.81 (d, J=5.2 Hz, 1H), 5.02 (s, 1H), 5.01 (dd, J=18.4, 1.6 Hz, 1H), 4.83 (dd, J=18.4, 1.2 Hz, 1H), 3.97 (brs, 1H), 3.84 (dq, J=9.6, 6.8 Hz, 1H), 3.57 (dd, J=9.6, 1.6 Hz, 1H), 2.78 (d, J=8.8 Hz, 1H), 2.15-2.11 (m, 2H), 1.88-1.39 (m, 17H), 1.29-1.22 (m, 6H), 0.93 (s, 3H), 0.87 (s, 3H); ¹³C NMR (100 MHz, CDCl₃) 174.8, 174.7, 129.7, 128.0, 117.8, 93.3, 85.8, 74.0, 73.6, 66.0, 60.6, 51.1, 49.8, 42.0, 40.2, 36.6, 35.9, 35.4, 33.3, 30.9, 30.5, 27.0, 26.9, 26.8, 23.9, 21.5, 21.3, 18.7, 16.0; ESIHRMS Calcd for [C₂₉H₄₁O₅H⁺]: 512.3124. Found: 512.3124.

### Synthesis of [2,3-dihydroxy-4-azido-α-L-Rhap]-Dig (B-3)

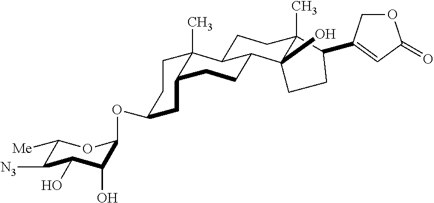

To a t-BuOH/acetone (290 μL, 1:1 (v/v), 0.2M) solution of digitoxin allylic azide B-2 (30 mg, 0.0586 mmol) at 0° C. was added a solution of N-methylmorpholine-N-oxide/water (50% w/v, 30 μL). Crystalline OsO₄ (0.75 mg, 5 mol %) was added and the reaction mixture was stirred for 8 h. The reaction mixture was quenched with 0.5 mL of saturated Na₂S₂O₃ solution, extracted with EtOAc (3×10 ml), dried over Na₂SO₄, and concentrated under reduced pressure. The crude product was purified via silica gel flash chromatography eluting with 1-2% MeOH/DCM to get sticky white solid B-3 (27 mg, 0.0495 mmol, 84%); R_{f} (10% MeOH/DCM)=0.25; ¹H NMR (400 MHz, CDCl₃): δ 5.87 (br 1H), 5.02 (d, J=18.4 Hz, 1H), 4.87 (s, 1H), 4.83 (dd, J=18.4, 1.5 Hz, 1H), 3.92 (brs, 1H), 3.90 (m, 2H), 3.64 (dq, J=10.4, 6.0 Hz, 1H), 3.31 (dd, J=9.6, 9.6 Hz, 1H), 2.78 (m, 1H), 2.53 (br, 1H), 2.35 (br, 1H), 2.15 (m, 2H), 1.88 (m, 2H), 1.72-1.21 (m, 20H) 0.92 (s, 3H), 0.87 (s, 3H); ¹³C NMR (100 MHz, CDCl₃) δ 174.8, 174.8, 117.9, 97.5, 85.8, 73.7, 72.2, 71.0, 70.8, 67.0, 66.4, 51.1, 49.8, 42.0, 40.2, 36.6, 35.9, 35.4, 33.3, 30.5, 29.6, 27.1, 26.7, 26.7, 24.0, 21.6, 21.4, 18.6, 16.0; ESIHRMS Calcd for [C₂₉H₄₃N₃O₂H⁺]: 546.3179. Found: 546.3179.

### Synthesis of [2,3-dihydroxy-4-amino-α-L-Rhap]-Dig (B-4)

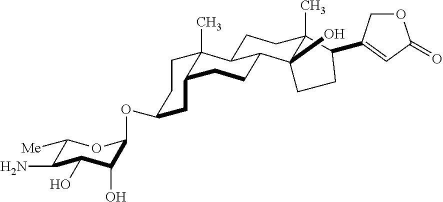

To a solution of azide diol B-3 (9 mg, 0.0165 mmol) in THF/H₂O (9:1, v/v, 0.16 mL) was added PPh₃ (10.8 mg, 0.041 mmol), then the mixture was stirred at room temperature for 6 h. The reaction mixture was evaporated with a little silica gel under reduced pressure and the crude product was purified with silica gel flash chromatography eluting with 10-12% MeOH/DCM to give C-4 amino rhamnose B-4 (7.1 mg, 0.0137 mmol, 83%); R_{f}=0.15 (15% MeOH/DCM); mp=125-132° C.; [α]²⁵_{D}=−22.6 (c=0.64, MeOH); ¹H NMR (400 MHz, CD₃OD): δ 5.90 (brs, 1H), 5.06 (d, J=19.2 Hz, 1H), 4.94 (s, 1H), 4.81 (s, 1H), 3.95 (brs, 1H), 3.70 (m, 2H), 3.64 (dd, J=10.4, 2.8 Hz, 1H), 2.83 (m, 2H), 2.21 (m, 2H), 1.91-1.26 (m, 19H), 1.24 (d, J=6.4 Hz, 3H) 0.96 (s, 3H), 0.88 (s, 3H); ¹³C NMR (100 MHz, CD₃OD) δ 178.5, 177.3, 117.8, 100.0, 86.4, 75.4, 73.6, 71.9, 71.7, 69.7, 55.7, 52.1, 51.1, 42.7, 40.9, 38.2, 36.8, 36.4, 33.4, 31.6, 30.8, 28.1, 27.9, 27.5, 24.3, 22.6, 22.4, 18.2, 16.4; ESIHRMS Calcd for [C₂₉H₄₅NO₇H⁺]: 520.3274. Found: 520.3270.

The following compounds were synthesized using the synthetic procedures describe above and were used for further assay and analysis. All of the compounds of Table 1 had the O-Dig (O-Digitoxigening) steroid core:

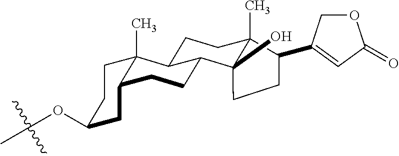

TABLE 1
Compounds having O-digitoxigenin core with varying sugar substituents

| Compound | | |
|---|---|---|
| No. | Glycoside residue | Structure |
| 1.1 | β-D-digitoxose mono- | 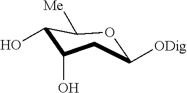 |
| 1.2 | α-L-rhamnoside mono- | 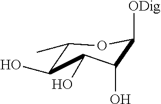 |
| 1.3 | α-L-rhamnoside di- | 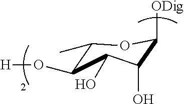 |
| 1.4 | α-L-rhamnoside tri- | 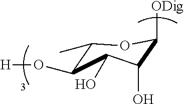 |
| 1.5 | α-L-amicetoside mono- | 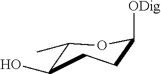 |
| 1.6 | α-L-amicetoside di- | 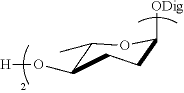 |
| 1.7 | α-L-amicetoside tri- | 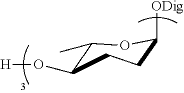 |

All of the compounds of Table 2 had the O-Dig (O-Digitoxigening) steroid and a α-L-rhamno or amiceto sugars substituted at the C-2, C-3 and C-4 position as shown below:

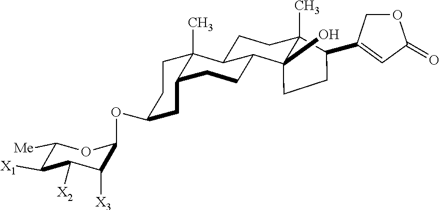

TABLE 2
Compounds having O-digitoxigenin core
with varying sugar substituents

| Compound No. | X₁ | X₂ | X₃ |
|---|---|---|---|
| 2.1 | NH₂ | OH | OH |
| 2.2 | NH₂ | H | H |
| 2.3 | OH | L-rhamnose | OH |
| 2.4 | OH | D-rhamnose | OH |
| 2.5 | OH | L-amicetose | OH |
| 2.6 | OH | D-amicetose | OH |
| 2.7 | D-rhamnose | OH | OH |
| 2.8 | L-rhamnose | OH | OH |
| 2.9 | OH | (1,3)-L,D-Dirhamnose | OH |
| 2.10 | OH | (1,3)-L,L-Dirhamnose | OH |

All of the compounds of Table 3 have a substituted O-Dig (O-Digitoxigening) steroid and α-L-rhamno or amiceto sugars substituted at the C-2 and C-3 position as shown below:

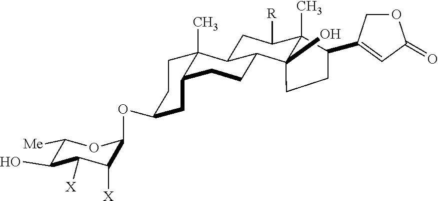

TABLE 3
Compounds having substituted O-digitoxigenin
core and varying sugar substituents

| Compound No. | X | R |
|---|---|---|
| 3.1 | H | OH |
| 3.2 | OH | OH |
| 3.3 | H | OH |
| 3.4 | H | OAc |
| 3.5 | H | OPiv |
| 3.6 | H | OTBS |
| 3.7 | OH | OH |
| 3.8 | OH | OAc |
| 3.9 | OH | OClAc |
| 3.10 | OH | OPiv |
| 3.11 | OH | OTBS |

### Example 5

### Structure Activity Relationship (SAR) Studies

### (A) Assay Procedures:

i. Cell Culture

Human non-small cell lung cancer cells (NCI-H460) were purchased from from the American Type Culture Collection (Manassas, Va.). NCI-H460 cells were cultured in RPMI 1640 medium supplemented with 10% bovine fetal serum (FBS), 2 mM L-glutamine and 100-units/ml penicillin/streptomycin. All experiments with NCI-H460 cells were performed in medium enriched with 1% FBS serum, 2 mM L-glutamine and 100-units/ml penicillin/streptomycin. 1% FBS was used due to existing concerns about digitoxin binding to serum proteins.

ii. Cell Viability Tests with MTT Assay

Cells were seeded overnight in 96 well plates at a concentration of 1×10⁴ cells/well, and then treated for 48 h with a log₁₀ scale dilution series of the glycosylated CS compounds (e.g., digitoxin or D6-MA) dissolved in sterile filtered DMSO. Subsequently, 10 μl of 5 mg/ml MTT reagent was added to each well and then incubated for 4 h at 37° C. Isopropanol acidified with 0.04 N HCl was used to dissolve converted dye. Absorbance at 570 nm was measured using an Automated Microplate Reader ELx800 (BioTek, Winooski, Vt.). Each experiment was conducted 4 times with 4 replicate wells per concentration.

iii. Trypan Blue Exclusion Assay

NCI-H460 cells were seeded overnight in 60 mm² dishes at 5×10⁵ cell/dish, and subsequently treated with 10 nM of the glycosylated CS compounds (e.g., digitoxin or D6-MA) for 24 h, 48 h and 72 h. After treatment, cells were collected, stained with 0.4% trypan blue, and counted using a Countess automated cell counter.

iv. Na⁺/K⁺ ATPase Activity Assay

Na⁺/K⁺ ATPase activity assay for release of inorganic phosphate was performed on Na⁺/K⁺ ATPase isolated from porcine cerebral cortex following exposure to each compound according to the manufacturer's protocol. Briefly, serial dilutions of each compound were prepared in a buffer containing 50 mM Tris, 25 mM MgCl₂, 0.5 mM ATP, 130 mM NaCl, and 20 mM KCl at pH 7.5, then plated in a 96 well plate in triplicate. Subsequently, diluted Na⁺/K⁺ ATPase was added to each well and the reaction allowed to proceed for 15 minutes. The reaction was stopped with Pi ColorLock Gold for 30 minutes, and then the absorbance of each well was determined at 595 nm.

α1/2/3-Na⁺/K⁺ ATPase pump binding data was assessed for compounds of Table 1.

v. Apoptosis Assay

Cells were seeded overnight in 12 or 24 well plates at a concentration of 1×10⁵ cell/ml and subsequently treated with different concentrations of the glycosylated CS compounds for 24 h. After treatment, cells were incubated with 10 mg/ml of Hoechst 33342 for 30 min and analyzed for apoptosis by scoring the percentage of cells having intensely condensed chromatin and/or fragmented nuclei using fluorescence microscopy (Leica Microsystems, Bannockburn, Ill.). Approximately 1,000 nuclei from ten random fields were analyzed for each sample. The apoptotic index was calculated as the percentage of cells with apoptotic nuclei over total number of cells.

vi. Cell Cycle Analysis

NCI-H460 cells were seeded in 60 mm² cell culture dishes at a concentration of 5×10⁵ cells/dish, starved overnight in serum-free media, and then treated with 1, 5, 10, and 20 nM of the glycosylated CS compounds for 48 h. Treated cells were then trypsinized, collected, washed with Phosphate buffered saline (PBS) and fixed in 70% ethanol at 4° C. overnight. Subsequently, cells were washed with PBS and stained with propidium iodide containing 0.05% RNase. For cell cycle analysis, the DNA content was determined using a FACScan laser flow cytometer (FACSCalibur; Becton Dickinson, San Jose, Calif.). Data were analyzed using MODFIT software (Verity Software House, Topsham, Me.). Experiments were repeated 4 times to conduct statistical analysis.

vii. Western Blot Analysis

Cells were seeded in 60 mm² cell culture dishes at a concentration of 1×10⁶ cell/plate, starved overnight, and then treated with 5 to 50 nM of each of the prepared compounds for 24 h. After treatment, cells were collected and lysed for 30 min on ice in lysis buffer containing 2% Triton X-100, 1% sodium dodecyl sulfate (SDS), 100 mM NaCl, 10 mM Tris-HCl (pH 7.5), 1 mM EDTA, and Complete Mini cocktail protease inhibitors. Insoluble debris was pelleted by centrifugation at 4° C. and 6800 g for 15 min. Subsequently, the supernatant was collected and used to determine protein content using BCA assay. Briefly, diluted supernatant samples and bovine serum albumin standards were plated in duplicate to a 96 well plate. Working reagent (1000 μL) was prepared by mixing 50 parts of reagent A (1000 μL) with 1 part of reagent B (20 μL), added to each well (200 μL each), and incubated at 37° C. for 30 mM. Absorbance of each well was measured at 562 nm with a Varioskan spectrophotometer (Thermo, Waltham, Mass.). BSA protein standard curves were plotted to determine sample protein content.

Samples were next separated on 12% SDS-PAGE and transferred to PVDF membranes using the iBlot® Dry Blotting System. Membranes were blocked in 5% skim milk in TBST (25 mM Tris-HCl, pH 7.4, 125 mM NaCl, 0.1% Tween 20) for 1 h, and subsequently incubated with appropriate primary antibodies at 4° C. overnight. Membranes were washed three times for 10 min each with TBST and then incubated with horseradish peroxidase-conjugated secondary antibodies for 2 h at room temperature. The immune complexes formed were detected by chemiluminescence (Supersignal West Pico; Pierce, Rockford, Ill.). Band quantification via densitometry was performed using ImageJ software version 10.2.

viii. Statistical Analysis

All results are presented as mean±standard deviation. For cell viability, ATPase activity and apoptosis assays, dose-response curves and concentrations that caused 50% effect (i.e. IC₅₀) were calculated for the prepared compounds listed in Tables 1-3 in all the cell lines tested using non-linear regression analysis in GraphPad Prism 5.0 (San Diego, Calif.). Two-way analysis of variance (ANOVA) and unpaired two-tailed Student's t-test with α=0.05 were performed to compare the effect of compounds and administered dose on cell viability, apoptosis and quantified protein expression data. Post-hoc Tukey-Kramer HSD tests were conducted on significant ANOVA results. Results were considered significant when p≦0.05.

ix. Anti-CMV Activity Assay

It has been observed that the infection of human foreskin fibroblasts (HFF) cells with HCMV causes an up regulation of the α3-isoform of the Na⁺/K⁺-Pump. Therefore the inhibition of HCMV replication by the compounds described herein was tested using procedure described by Cai, H. et al., in Med. Chem. Lett. 2014, 5, 395-399, which is incorporated by reference in its entirety. Generally, inhibition of Towne HCMV: pp28-luciferase activity was measured in cell lysates of HCMV-infected HFFs collected at 72 h post infection (hpi). Virus DNA yield in supernatants of HCMV-infected cells collected at 96 hpi was measured by real-time PCR. Plaque reduction assay performed at 8 days postinfection. Data represent mean values (±SD) of triplicate determinations from three independent experiments.

Inhibition of TB40 HCMV: HFFs were infected with HCMV-TB40 strain at MOI of 1 pfu/cell and treated with compounds listed in Tables 1-3 for 3 days.

### (B) Results:

### (i) Na⁺/KATPase Enzyme Activity

The α1/2/3-Na⁺/K⁺ ATPase pump binding data and anticancer data (MTT and Apoptosis) for the compounds of Table 1 is depicted the Table 4.

TABLE 4
Na⁺/K⁺ATPase and Anticancer Data for Compounds of Table 1

| | Na⁺/K⁺ATPase inhibition | | | Anticancer | |
|---|---|---|---|---|---|
| | K_{D}, nM | | | MTT | Apoptosis |
| | α1β1 | α2β1 | α1β1 | IC₅₀ | GI₅₀ |
| Cardiac glycoside | | | | | |
| Digitoxin | 80 | 38 | 36 | 357 | 11 |
| β-D-digitoxose | | | | | |
| 1.1: mono- | — | — | — | 75 | 4 |
| α-L-rhamnoside | | | | | |
| 1.2: mono- | 27 | 23 | 19 | 47 | 2 |
| 1.3: di- | — | — | | 365 | — |
| 1.4: tri | 393 | 164 | 345 | 1347 | — |
| α-L-amicetoside | | | | | |
| 1.5: mono- | 56 | 38 | 26 | 48 | 3 |
| 1.6: di- | 223 | 156 | 186 | 510 | — |
| 1.7: tri- | 670 | 149 | 736 | 3963 | — |

From the studies, it was observed that amongst the tested compounds, O-glycosides with β-D-digitoxo-, α-L-amiceto-, and α-L-rhamno-stereochemistry were the most active. It was also observed that regardless of the glycosidic linkage (O-/neo- and/or α-/β-) and sugar stereochemistry, the monosaccharides were more effective than di- and trisaccharides. Further, substitution of the C6′-position on the α-L-rhamno and α-L-amiceto-sugars was found to be deleterious to cancer cell cytotoxicity. This shows that the modification of the carbohydrate portion of the cardiac glycosides in the compounds is such that it improves the anti-cancer activity.

Further, from the results, a strong correlation between cancer cell cytotoxicity and α1/3-Na⁺/K⁺ ATPase pump binding selectivity was observed (see: α-L-amiceto-mono-, di- and tri-saccharides; Scheme 1). In addition, it was observed that mono-saccharides have improved activity and α1/2/3-isoform selectivity. In addition, it was also demonstrated that the compounds bind to the three α-isoforms (α1β1/α2β1/α3β1) of the Na⁺/K⁺ ATPase (e.g., compounds 1.2, 1.4-1.7; Table 1) in a radioactive obtain displacement assay. The Na⁺/K⁺ ATPase binding data also correlates with the cytotoxicity data, which can be seen in the binding data of compounds 1.5-1.7 to all three isomers (α1β1/α2β1/α3β1), which decreases proportionally the MTT data (Table 1). This observation strongly suggests that binding to the Na⁺/K⁺ ATPase in cancer cells is the origin of its anti-cancer activity.

### (ii) NCI-H460 Cell Viability

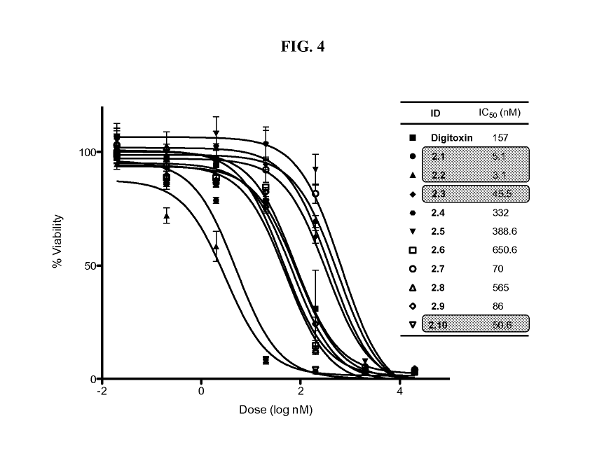

The dose-response curve for the digitoxin analogues having varying substitution on the sugar molecule are depicted in FIG. 4 and the IC₅₀ values for these compounds are summarized in Table 5.

TABLE 5
IC₅₀ Data for Compounds of Table 2

| ID | IC₅₀ (nM) |
|---|---|
| Digitoxin | 157 |
| 2.1 | 5.1 |
| 2.2 | 3.1 |
| 2.3 | 45.5 |
| 2.4 | 332 |
| 2.5 | 388.6 |
| 2.6 | 650.6 |
| 2.7 | 70 |
| 2.8 | 565 |
| 2.9 | 86 |
| 2.10 | 50.6 |

From the results, it was observed that the C4 amino-substituted compounds (2.1 and 2.2) exhibited improved cytotoxicity for H460 cells. It was also observed that the mixed D/L-disaccharide (2.7), with 1,4-linkage has much greater activity than the mixed L/L-disaccharide (2.8). Similarly promising L/D-selectivity was discovered for substitution of the monosaccharide at C3, where substitution with another α-L-rhamnose ring does not significantly reduce activity (i.e., disaccharide 2.3) in contrast to a D-sugar (i.e., disaccharide 2.4). Moreover, a subsequent substitution at the C3-position with another L- or D-rhamnose rings showed similar selectivity (i.e., trisaccharides 2.9 vs. 2.10) but with minimal loss in activity. This L-/D-sugar selectivity indicates that the mono- di- and tri-saccharide portions of these analogs are all still in the carbohydrate binding region.

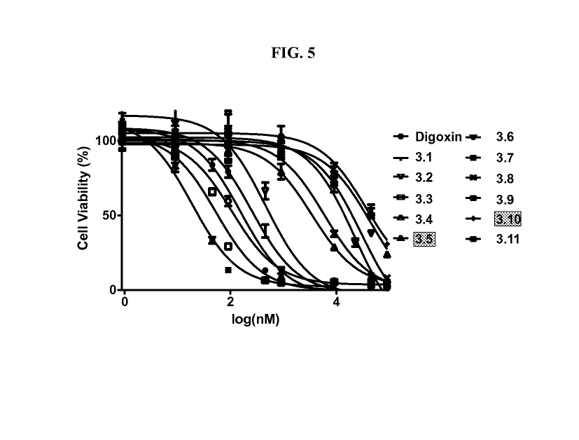

It was determined that cancer cell selectivity can also be introduced via substitution of the C-ring of the aglycon (i.e., digoxin). The dose-response curve for the digitoxin analogues having varying substitution on the C-ring of the steroid are depicted in FIG. 5 and the IC50 values for these compounds are summarized in Table 6.

TABLE 6
IC₅₀ Data for Compounds of Table 3

| ID | IC₅₀ (nM) |
|---|---|
| Digitoxin | 157 |
| 3.1 | 253 |
| 3.2 | 486 |
| 3.3 | 54 |
| 3.4 | 3445 |
| 3.5 | ≧50000 |
| 3.6 | 29724 |
| 3.7 | 19 |
| 3.8 | 5977 |
| 3.9 | 105 |
| 3.10 | ≧50000 |
| 3.11 | 2249 |

From the results, it was observed that substituting the C-ring with large groups (i.e., OPiv as in 3.5 and 3.10) greatly reduced activity. Thus, the C-ring substitution site is an ideal position for the introduction of prodrug Co-NO₂Bn) and photo-affinity (o-NO₂Bn) carbonate groups, which was accomplished by substituting the digitoxigenin to digoxigenin, which showed a similar carbohydrate substitution effect where the α-L-rhamno- and amiceto-sugars (3.7 and 3.3) were more active than the diastereomeric α-D-sugars (3.2 and 3.1).

(iii) Anti-CMV Activity

The glycosylated cardiotonic steroid compounds were tested for their anti-cytomegalovirus (CMV) activity. Specifically, the compounds were tested for the ability to inhibit HCMV replication. For each compound, the anti-HCMV activity in infected HFFs was expressed as EC₅₀, whereas cytotoxicity in noninfected HFFs was expressed as CC₅₀. Selectivity index (SI), defined as CC₅₀/EC₅₀, was calculated for each compound. The EC₅₀, CC₅₀, and Selectivity Index (SI) values for exemplary compounds is listed in Table 7 below.

TABLE 7
EC₅₀, CC₅₀ and selectivity index (SI) of digitoxin analogs

| Compound | EC₅₀ (nM) | CC₅₀ (nM) MTT | SI |
|---|---|---|---|
| Digitoxin | 23.33 ± 0.67 | 2810.6 ± 668.0 | 120.46 |
| α-L-Amicetose (1.5) | 3.77 ± 0.08 | 654.7 ± 177.0 | 173.61 |
| Bis-α-L-Amicetose | 14.78 ± 0.44 | 1119.7 ± 140 | 75.77 |
| Tris-α-L-Amicetose | 113.37 ± 2.86 | 2370.6 ± 278.3 | 20.91 |
| α-D-Amicetose | 37.50 ± 0.52 | 1105.05 ± 272.93 | 29.46 |
| Bis-α-D-Amicetose | 785.57 ± 37.28 | 12096.2 ± 718.3 | 15.40 |
| Tris-α-D-Amicetose | 2081.8 ± 93.4 | 14602.4 ± 1018.5 | 7.01 |
| α-L-Rhamnose (1.2) | 4.77 ± 0.23 | 664.2 ± 184.6 | 139.25 |
| Bis-α-L-Rhamnose | 36.69 ± 0.91 | 1496.4 ± 573.4 | 40.78 |
| Tris-α-L-Rhamnose | 232.75 ± 3.88 | 6952.4 ± 859.6 | 29.87 |
| α-D-Rhamnose | 26.57 ± 0.88 | 2272.7 ± 551.2 | 85.52 |
| Bis-α- -Rhamnose | 209.59 ± 5.98 | 6739.3 ± 717.4 | 32.15 |
| Tris-α-D-Rhamnose | 321.87 ± 11.67 | 9968.7 ± 1318.3 | 30.97 |
| α-D-Mannose | 7.31 ± 0.12 | 927.5 ± 376.9 | 126.79 |

From the data, it was observed that, as with the cancer cell cytotoxicity, the L-isomers showed improved anti-HCMV activity compared to the D-isomers, as well as, an inverse correlation between the sugar chain length and anti-HCMV activity was observed. Virus yields were determined for these compounds, and a dose dependent effect on virus DNA yield was observed. While the selectivity (SI>100) for infected cells are high, these selectivities are being achieved with pan-Na⁺/K⁺ ATPase inhibitors. When compared to the anti-HCMV data in Table 7, a similar trend in improved cytotoxicity is seen in cell infected with HCMV. Importantly, this improvement in activity also comes with a modest improvement in the selectivity index (SI) for infected and cancer cells over normal cells. So, even without affecting the isoform selectivity, it was possible to significantly improve the potency and the selectivity index for two new analogs 1.2 and 1.5 (SI of 173 and 139, respectively) versus digitoxin (SI of 120), a prescribed drug for congestive heart failure.

### (iv) Summary of SAR Studies for Anti-Cancer Activity:

From the studies, it was observed that, (a) reducing the 1,4-trisaccharide to a monosaccharide improves activity (˜5 fold), (b) only the anomeric stereocenter (β-D and α-L have the same anomeric configuration) is conserved for activity, (c) larger 1,3-linked di- and tri-saccharides only have a slight reduction in activity, and (d) a C4 amino-substitution significantly improves activity (>10 fold).

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A compound which is a glycosylate of an A-ring of a cardiotonic steroid, wherein the steroid is attached to the anomeric position of (a) a monosaccharide comprising a C-4 amino group, or (b) an oligosaccharide.

2. The compound of claim 1 that is represented by the following formula I: 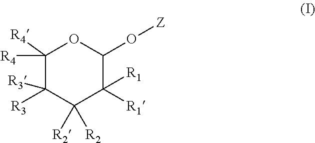
wherein:
each of R₁, R₁′, R₂, and R₂′ independently, is H, OH, alkyl, alkenyl, alkynyl, aryl, alkoxyl, cycloalkyl, heterocycloalkyl, heteroaryl, O-aryl, O-monosaccharide, O-oligosaccharide;
each of R₃ and R₃′, independently, is H, OH, alkyl, alkoxyl, cycloalkyl, alkenyl, alkynyl, aryl, cycloalkyl, heterocycloalkyl, heteroaryl, O-aryl, O-monosaccharide, O-oligosaccharide, and NR₂₀R₂₁, R₂₀ and R₂₁ being H, alkyl, alkenyl, alkynyl, aryl, cycloalkyl, heterocycloalkyl, heteroaryl;
each of R₄ and R₄′, independently, is H, alkyl, alkenyl, alkynyl, aryl, alkoxyl, cycloalkyl, heterocycloalkyl, and heteroaryl, or R₄ and R₄′ together with the carbon atom they attach to form a cycloalkyl or heterocycloalkyl ring; and
Z is a cardiotonic steroid aglycon.

3. The compound of claim 1, wherein R₃ is H or alkyl and R₃′ is NR₂₀R₂₁.

4. (canceled)

5. The compound of claim 3, wherein R₁ is H or OH.

6. The compound of claim 4, wherein R₂ is H, OH, or O-monosaccharide.

7. The compound of claim 1, wherein R₂ or R₂′ is O-monosaccharide, O-disaccharide, or O-trisaccharide.

8-10. (canceled)

11. The compound of claim 1, wherein Z is represented by formula II: 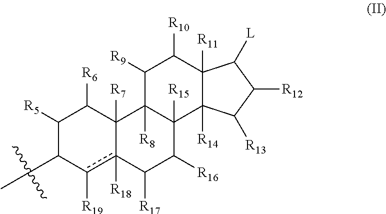
wherein: [symbol]
indicates a single or a double bond;
each of R₅, R₆, R₇, R₈, R₁₁, R₁₃, R₁₄, R₁₅, R₁₆, R₁₇, R₁₈, and R₁₉, independently, is H, OH, carbonyl, alkyl, alkoxyl, acyloxy, carboxy, alkylcarboxy, hydroxyalkyl, —C(O)R₂₂, or two adjacent groups together with the bond that the two groups attach to form an epoxide ring;
R₂₂ is H or alkyl;
each of R₉, R₁₀, and R₁₂, independently, is H, OH, carbonyl, or a cleavable prodrug group; and
L is a heterocyclic ring.

12. The compound of claim 11, wherein R⁷ is H, —OH, CH₃, CH₂OH, C═O, C(O)H, —OC(O)H, or —OC(O)alkyl.

13. The compound of claim 11, wherein R¹⁰ is H, —OH, CH₃, or CH₂OH.

14. The compound of claim 11, wherein R¹¹ is H, —OH, CH₃, or CH₂OH.

15. (canceled)

16. The compound of claim 11, wherein L is a lactone.

17-18. (canceled)

19. The compound of claim 16, wherein L is represented by formula III: 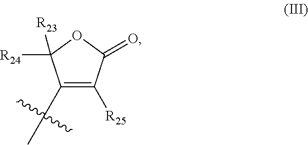
wherein:
each of R₂₃, R₂₄, and R₂₅, independently, is H, halo, alkyl, alkenyl, alkynyl, alkoxyl, cycloalkyl, aryl, carboxy, alkylcarboxy, amino, alkylamino, or dialkylamino.

20-21. (canceled)

22. The compound of claim 16, wherein L is represented by formula IV: 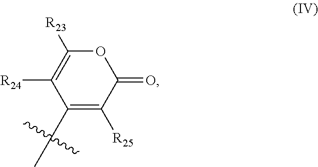
wherein:
each of R₂₃, R₂₄, and R₂₅, independently, is H, halo, alkyl, alkenyl, alkynyl, alkoxyl, cycloalkyl, aryl, carboxy, alkylcarboxy, amino, alkylamino, or dialkylamino.

23. (canceled)

24. The compound of claim 22, wherein Z is arenobufagin, bufalin, bufatonin, telocinobufagin, cinobufagin, marinobufgin, proscillaridin aglycon, or scilliroside agylcon.

25. The compound of claim 11, wherein Z is digitoxigenin.

26. The compound of claim 11, wherein Z is digoxigenin.

27-28. (canceled)

29. The compound of claim 2, wherein Z is other than digitoxin, oleandrin, digitoxin-β-D-digitoxose, or digitoxin-mono-α-L-rhamnoside.

30. A pharmaceutical composition comprising a compound of claim 1 and a pharmaceutically acceptable excipient.

31. A method of inhibiting a Na/K-ATPase pump, comprising contacting the Na/K-ATPase pump with an effective amount of the compound of claim 1.

32. A method of treating congestive heart failure in a subject comprising administering to the subject an effective amount of the pharmaceutical composition of claim 30.

33-34. (canceled)
